SEQUENCE LISTING (155 sequences)

SEQ ID NO: 1 - protein, 147 aa, Homo sapiens
  MISC_FEATURE (1)..(20): Signal Peptide
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 147 aa, Homo sapiens
  MISC_FEATURE (1)..(20): Signal Peptide
MASHRLLLLCLAGLVFVSEAGPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 20 aa, Homo sapiens
MASHRLLLLCLAGLVFVSEA

SEQ ID NO: 4 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 5 - protein, 127 aa, Homo sapiens
GPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 6 - DNA, 444 bp, Artificial Sequence
  Synthetic
  misc_feature (1)..(60): Signal Peptide
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 7 - DNA, 444 bp, Artificial Sequence
  Synthetic
  misc_feature (1)..(60): Signal Peptide
atggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccatgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 8 - DNA, 60 bp, Artificial Sequence
  Synthetic
atggcttctcatcgtctgctcctcctctgccttgctggactggtatttgtgtctgaggct

SEQ ID NO: 9 - DNA, 384 bp, Artificial Sequence
  Synthetic
ggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 10 - DNA, 384 bp, Artificial Sequence
  Synthetic
ggccctacgggcaccggtgaatccaagtgtcctctgatggtcaaagttctagatgctgtccgaggcagtcctgccatcaatgtggccatgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatga

SEQ ID NO: 11 - DNA, 938 bp, Artificial Sequence
  Synthetic
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 12 - DNA, 7258 bp, Homo sapiens
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccgtgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaa

SEQ ID NO: 13 - protein, 147 aa, Mus musculus
  MISC_FEATURE (1)..(20): Signal Peptide
MASLRLFLLCLAGLVFVSEAGPAGAGESKCPLMVKVLDAVRGSPAVDVAVKVFKKTSEGSWEPFASGKTAESGELHGLTTDEKFVEGVYRVELDTKSYWKTLGISPFHEFADVVFTANDSGHRHYTIAALLSPYSYSTTAVVSNPQN

SEQ ID NO: 14 - protein, 20 aa, Mus musculus
MASLRLFLLCLAGLVFVSEA

SEQ ID NO: 15 - protein, 127 aa, Mus musculus
GPAGAGESKCPLMVKVLDAVRGSPAVDVAVKVFKKTSEGSWEPFASGKTAESGELHGLTTDEKFVEGVYRVELDTKSYWKTLGISPFHEFADVVFTANDSGHRHYTIAALLSPYSYSTTAVVSNPQN

SEQ ID NO: 16 - DNA, 444 bp, Artificial Sequence
  Synthetic
  misc_feature (1)..(60): Signal Peptide
atggcttcccttcgactcttcctcctttgcctcgctggactggtatttgtgtctgaagctggccccgcgggtgctggagaatccaaatgtcctctgatggtcaaagtcctggatgctgtccgaggcagccctgctgtagacgtggctgtaaaagtgttcaaaaagacctctgagggatcctgggagccctttgcctctgggaagaccgcggagtctggagagctgcacgggctcaccacagatgagaagtttgtagaaggagtgtacagagtagaactggacaccaaatcgtactggaagacacttggcatttccccgttccatgaattcgcggatgtggttttcacagccaacgactctggccatcgccactacaccatcgcagccctgctcagcccatactcctacagcaccacggctgtcgtcagcaacccccagaattga

SEQ ID NO: 17 - DNA, 60 bp, Artificial Sequence
  Synthetic
atggcttcccttcgactcttcctcctttgcctcgctggactggtatttgtgtctgaagct

SEQ ID NO: 18 - DNA, 384 bp, Artificial Sequence
  Synthetic
ggccccgcgggtgctggagaatccaaatgtcctctgatggtcaaagtcctggatgctgtccgaggcagccctgctgtagacgtggctgtaaaagtgttcaaaaagacctctgagggatcctgggagccctttgcctctgggaagaccgcggagtctggagagctgcacgggctcaccacagatgagaagtttgtagaaggagtgtacagagtagaactggacaccaaatcgtactggaagacacttggcatttccccgttccatgaattcgcggatgtggttttcacagccaacgactctggccatcgccactacaccatcgcagccctgctcagcccatactcctacagcaccacggctgtcgtcagcaacccccagaattga

SEQ ID NO: 19 - DNA, 1237 bp, Artificial Sequence
  Synthetic
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggatggcttcccttcgactcttcctcctttgcctcgctggactggtatttgtgtctgaagctggccccgcgggtgctggagaatccaaatgtcctctgatggtcaaagtcctggatgctgtccgaggcagccctgctgtagacgtggctgtaaaagtgttcaaaaagacctctgagggatcctgggagccctttgcctctgggaagaccgcggagtctggagagctgcacgggctcaccacagatgagaagtttgtagaaggagtgtacagagtagaactggacaccaaatcgtactggaagacacttggcatttccccgttccatgaattcgcggatgtggttttcacagccaacgactctggccatcgccactacaccatcgcagccctgctcagcccatactcctacagcaccacggctgtcgtcagcaacccccagaattgagagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaaaaaaaaaaaaaaaaaa

SEQ ID NO: 20 - DNA, 9077 bp, Mus musculus
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacaggatggcttcccttcgactcttcctcctttgcctcgctggactggtatttgtgtctgaagctggccccgcggtgagtgatcctgtgagcgatccagacatggcagttagaccttagataaagaagaagtgccttcttccagatgtgagaactagagtactcagactctatatttaccattagactccaaagagaagagctggagtgcctctggctcttccttctattgctttagcgcattgggtctgtagtgctcagtctctggtgtccttagataataaagatatgagattaacatagaaataaagatataaaagggctggatgtatagtttagtggtccagtgtatgcctagtatgtgaaaagccttctgttcaacctctagcaatagaaaaacaagatatattctcggtggggctgttaatattgaattctcataaaatctttaatatatttagtatgcctattatgttgttatattttagttctttagctaatcaaaatgcattattgatctttctttgtctttttttggccaacactctattccagtctttgaaaaagtcctttaaaagagttaatcagtataattaaatgagtcaggaagtatgtgagggttattttacaaccagagggaattactatagcaacagctgattagaatgatctcaagaaaaagcccattctgtctttttgcaccatgcacctttcagtggctccattcagatggagaggcaaacagagcaatggctctcagagggcctattttccctttgaacattcattatccatatccctggtgcacagcagtgcatctgggggcagaaactgttcttgctttggaaacaatgctgtctatgtcatactggataaagaagctcattaattgtcaacacttatgttatcataatgggatcagcatgtacttttggttttgttccagagtctatcaccggaaagaacaagccggtttactctgacccatttcactgacatttctcttgtctcctctgtgcccagggtgctggagaatccaaatgtcctctgatggtcaaagtcctggatgctgtccgaggcagccctgctgtagacgtggctgtaaaagtgttcaaaaagacctctgagggatcctgggagccctttgcctctgggtaagcttgtagaaagcccaccatgggaccggttccaggttcccatttgctcttattcgtgttagattcagacacacacaacttaccagctagagggctcagagagagggctcaggggcgaagggcacgtattgctcttgtaagagacacaggtttaattcctagcaccagaatggcagctcataaccatctgaaactcacagtcttaggagatctgggtatctgacattctcttctacccaccatgtgtgtggtgcacaaattcacatgcaggcatcaaatcttataaacaacaacaaaaaaccaacaaacctggtagcaaaagaagattagaaggttaaacatatgagccgagagcttttgttttgttttgttttgttttgttttgtttacatttcaaatgttatcccctttctcggtccccctccccaaaccctctaccccattctctcctccccttcttctatgagggtgttccccaccaacccactcccaccttcctgctctcgaattcccctatactgggacatcaagccttcacagaatcaagggcctctcctcccattgatgcccgacaatgtcatcctctgctacctatgtggctggagccatgggtcccttcatgtatcctccttggttggtggtttagtctctgggaggtctgggggatctggttgattgatattattgttcttcctatgagattgcaaaccccttcagctccttcggtcctttaactcctccactggggaccccgagctcagtccaatggttggctgtgagcatccaccagcagaggcctttttttttttttttaacaaagctgctttattatgttgcttagagcatgaccaggaaccagagcacagtccaagactgaagggaggaaaagggggggagtcaataaccccactgtttcatagtggtttgcaacccttttatatcacagcccactttaggcaaataatgaaaattatagtctccagggacagagaagatggtgcaggaagtgaagtgcctgctcagaaaatgggggcttgaatgtgagttcccagactctgtgtaagatgcccagcatcgaagtgcatgcttataacaccagcctggaggtagaagcttagaaacaggggtaccctgaagttgcttgttcaccagtgtccctgaatgggtaggtgcatgtttggtgagagaccctgtctcaaaaatcaaggtgtaggataattgaaaatacctagctttgagcttagatcatgcaaatgtgtacacacactcacacacaccacacacacaaaaaaatgcagagacagagagatacagagagacagagagatacagagacagagacagagagaaaaggagaaagtaaaaaacaaataatttaaagacccatggccacaaagaggctcaaagacaagcacgtataaaaccatacacatgtaattttaggagttttcagattccctggtacccgtgggtgatgcacaagctttgaatcccagtcttaaaatcttacgaagaacgtgttcgtgtgtgctaatttattgatgagaggaaaggaattgacaaagtgcccttccggagcttcctgcattacccagactcagggtttttttaaatgtacactcagaacagagtagctctgtgcaagggtagcaaccacgaagcttaataagaaacatatcgtgagagatctgcaaggcaaatctaggggctgaccaatctcacagtcacccactagcatgtcaacacaacttcccacctgtgctagccacttagcaattttgtgttgttctgttttgtttttgtttttaacaaagcaatttcaaagagatttctaattcatctaaacaaacaaaccaaaaggaaaacagcaaagacgccctgagcacttagcagagcagctatgcagttatgactcctgggtggagactttatatcaggcttcaactgaatacctagaacctactagtgctcttcatcaatccttgggaaggtcattttcttttggtgctgttttgagtttctatttgttaatgtcttcataattatacacgtgttgagcacagcatgcaaagtgattaggggaatctagttggagtggaatggatacccaaatattcagactttcttgtgactcttctttcttgtacccacatcaaaaaaaaaaaaaatggagatgagacatggtcagagtcactaaaaccagctgctacttttaattacgtggggagcagtttctaacattgccattattgaactgatgctgcctgggtggaaatggaaatcacttagtatttcttgttggcaaagaattactgaatggattaaatttccaaagggagaagtcagttacaagtcttttctttgtttattaggctttctgctatgataaattacactacttccagaagttacccttaggccatgggacactggactatcactctgctgtcacaagagattacagagttagtcaaggcagcttgtgacaccttcagggactgtcataaacttccagcaagtcattaatcctgaatgcaatactgtgtgtgtgtgtctatgtgtgtttgtatgtctgtgtgtgtcttatgtctgtgtctctgtgtgtgtgtgtgtttgtgtgtgtgtgtgtatgtatgcctgtgtgtgtcttatgtctgtgtttgtgtgtctgtgtgtgtcttatgtctgtgtttgtatgtctgtgtgtgtctgtgtgtgtcttatgtctgtgtctctgtgtgtgtgtgtgtgtatgtatgtatgtatgtatgtatgtgtatgtgtttgcatctctctgtgtgtctgcgcttatatatttgtgtatgtgtttatgtgttcgcctttgtgcgttgttggggattgaatccaggggaatacaaatgttaagaaagaacgttaccactaagcttcacctgtaggccttaaagcttttctttcttttaaaaattgtaattaattcattttcagtcaggatctccacacctcgtccctgctgctctagaactcactatttaaacacaatcgccctcaaacctgcagcaaccctcccgcctctaccctgcgagcactagaataataacaggtgaccccacacgcctagattaagacctttaaggtaaacattttactatattttagtctcataagacaagatgctacaataaagctgtacataaagttccctcgaatttcttgctattttaactcaaacataaggatttcctcctttttgattcaggtaacagaaaaaatacacaggtacatacatgtacacacatgaacacacacgcatcacaaccacatatgcgcacgcttgtgtgatctatcatttaccatgccactgaactcttctttccccataaattcctctggacttgtgtgccctccaggaagaccgcggagtctggagagctgcacgggctcaccacagatgagaagtttgtagaaggagtgtacagagtagaactggacaccaaatcgtactggaagacacttggcatttccccgttccatgaattcgcggatgtaagtggacacaccaagttgtttggattttgtttttagtctcaggaaattcccttcgctcttgctgtacgatgggcatgagtggaaagtagattccacagccagaatccacagtgctgggaaagcaagccttctgaatttttctaaaactcatttagcaacatggcctgaacctgttcacactgcttatggtcagctaactatatttatgtaaatattcatttctctgttgaggaaatgttagtatttgcttttgaggcaacctccagataccatggagggcatgtcatagtcaaagagagggctccctatggtatttctctaaattctggcatttcctttattccaaagcacatctagtgtccccagaagtttgggtagacaattcttggcaacacagagaattacaacatgttcaaaacccaacagcttaatatctaaatcatcaagcaaacatcacatggcaaagggatttctgaatcaaaactgtttcatccttatgatcaacctatggaggtctagcctcgacttacacccattttaccaataagctaagagaagctaagttcctcatcaaggacacaaggctagcatgtgtgagcaagtgacagagttgccctctatgttggttagtgtgccttagccagtgtctcagtaagaaatggagctaaatcaaaacccaaggccaacagccaaaggcacatgagtaacctttgcttggcactgggctcagtttccctggctcctctcagtcctcagttcacagaggcagctgtcatgcaaatagaatccaagcttgttggtcagacctggagataacaaattccatcaaaaatagctcctcatgtgacctagtttgctgtctgttgctatgatacacaccatgaccgaaaagcaaccctggggagagaagggtttatttcatcttacagcttacagttcaccatggaggaaagccaggtgggaacctggaagtggaaattgaagcagagaccagaaaggaatgctgtttactggctggcttagctccttttcttatacagcttaggtctatgtgcccaggggatggtactgccgagcataggctgagcccgcctacatcaaccattagtcaaaaaaaggtccatagacttgcctacaggccaatctcatggaggcaataccccagtggagggtccctcttcgcaggttactctagtttgtgtcaagttgacaaaacctaaccacaaagcacaaacagggtctgcccttgtggcttagccatggatgacactctcagatgatggtgttaccagacaaaccagaggggctcaccaagagtctgccacctaccaaggtagtactctactcctcactgggcaccaacacccatattagctgggccagtacaggacccttgctgtttcctgcatgaattgtccatagaccctgggtctcagcctgccgggagtacctgtaagtagtcgcctcaaacacattattcctgttggaagacttgtctgattctcttttagaactcaatcaacaaacgtttttattttgttttggctttttggagacaagatctctcataggccagcctgacttgaatgtagctgaggatgacctgtgctgctaatcttctcgcctcttcctcccaagtggtaggataataggcataagacaccacagcagttttactccataccagggctctgaacccagactttaaacactctatcaactgattcacattcccaccccatcattcaacaaacatttgaaaaataaaacccttctgccttgagcactctgctaaatacagcctttgagtgcggagtatttcctcacaaccagggtccaagatgaccccatcatacataccacggaaaattaggagatgtttttaggtctctttgcttggggtaatttttatgtgtgtgtgtacacagccctgtgcgtgtgtgtgtgtgtgtgtgtgtgtgtgtacaggcacacacgtgtatgcatgtagaggctacataaaaaccttaggtgtcattctcaggcactctgttcaccccttcacacagcccgaacacacaaaatttgaggcattagcctggagctcaccagttaggctagactgacttgccagcagaccccaggctgtctccatctccccagctctgggattacaaactctatcataccagacatttttatacatattctgagcataaaattcatgtcttcaggctaacaagtcaagagcttaaatgactgagctctcttacgtggtggattttttttaaaactacataatatcttttttttttttttcacttctggggaagaaacaaatgagcctgagtgacaatgcgacagaaaagaaattttgaggagtgtgtgtgtctgtgtgtgtggtggcacatgcctctcatctaatgctagaggctacagtagaatgctcctgaattagtggccagccaaggccaagggctagggttgtaactcagtggcagagggcttgcctagcattcgcaggatttgatccatagcgctataaataataataaataaatacaacagtctaagatgattctccctttcatttatctggatgttatttttgtgttagttttactctgtcatccaatcattgtttgccctatatttggacatttaaaaaaaatctttattccaagtgtgttcaaagctgtatccaaaacctgtccaccaaatgagtccaatgacatacatcttctatattaccatctgttccagatttggctgactcccggcacctgggctgttgctgcacccatgtctcagatagtctagtgatttgagaagtgactagtaattgcaaaatccagactttgtccagaaacttctatgagctccaaaactttcatttacatttctgccagccacaaaccgcttgtgttgtggagagaaccctgtgatgtcttcccacagcatctcagccttgtttcttcccttaaaatattcatcttttcacattagaacatgcaaagggacagtgggagcgaaacccctggactgggacgcacgaagccttcctttctggtcaggctctcactgtagaaacttaggccggtttcagcatgcagtctgctggagaatggctcctgccaacattccaggtctggaagtttgtagtggagttgttgataaccactgttcgccacaggtcttttgtttgtgggtgtcagtgtttctactctcctgacttttatctgaacccaagaaagggaacaatagccttcaagctctctgtgactctgatctgaccagggccacccacactgcagaaggaaacttgcaaagagagacctgcaattctctaagagctccacacagctccaaagacttaggcagcatattttaatctaattattcgtcccccaaccccaccccagaggacagttagacaataaaaggaagattaccagcttagcatcctgtgaacactttgtctgcagctcctacctctgggctctgttagaactagctgtctctcctctctcctaggtggttttcacagccaacgactctggccatcgccactacaccatcgcagccctgctcagcccatactcctacagcaccacggctgtcgtcagcaacccccagaattgagagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaa

SEQ ID NO: 21 - DNA, 8300 bp, Mus musculus
atggcttcccttcgactcttcctcctttgcctcgctggactggtatttgtgtctgaagctggccccgcggtgagtgatcctgtgagcgatccagacatggcagttagaccttagataaagaagaagtgccttcttccagatgtgagaactagagtactcagactctatatttaccattagactccaaagagaagagctggagtgcctctggctcttccttctattgctttagcgcattgggtctgtagtgctcagtctctggtgtccttagataataaagatatgagattaacatagaaataaagatataaaagggctggatgtatagtttagtggtccagtgtatgcctagtatgtgaaaagccttctgttcaacctctagcaatagaaaaacaagatatattctcggtggggctgttaatattgaattctcataaaatctttaatatatttagtatgcctattatgttgttatattttagttctttagctaatcaaaatgcattattgatctttctttgtctttttttggccaacactctattccagtctttgaaaaagtcctttaaaagagttaatcagtataattaaatgagtcaggaagtatgtgagggttattttacaaccagagggaattactatagcaacagctgattagaatgatctcaagaaaaagcccattctgtctttttgcaccatgcacctttcagtggctccattcagatggagaggcaaacagagcaatggctctcagagggcctattttccctttgaacattcattatccatatccctggtgcacagcagtgcatctgggggcagaaactgttcttgctttggaaacaatgctgtctatgtcatactggataaagaagctcattaattgtcaacacttatgttatcataatgggatcagcatgtacttttggttttgttccagagtctatcaccggaaagaacaagccggtttactctgacccatttcactgacatttctcttgtctcctctgtgcccagggtgctggagaatccaaatgtcctctgatggtcaaagtcctggatgctgtccgaggcagccctgctgtagacgtggctgtaaaagtgttcaaaaagacctctgagggatcctgggagccctttgcctctgggtaagcttgtagaaagcccaccatgggaccggttccaggttcccatttgctcttattcgtgttagattcagacacacacaacttaccagctagagggctcagagagagggctcaggggcgaagggcacgtattgctcttgtaagagacacaggtttaattcctagcaccagaatggcagctcataaccatctgaaactcacagtcttaggagatctgggtatctgacattctcttctacccaccatgtgtgtggtgcacaaattcacatgcaggcatcaaatcttataaacaacaacaaaaaaccaacaaacctggtagcaaaagaagattagaaggttaaacatatgagccgagagcttttgttttgttttgttttgttttgttttgtttacatttcaaatgttatcccctttctcggtccccctccccaaaccctctaccccattctctcctccccttcttctatgagggtgttccccaccaacccactcccaccttcctgctctcgaattcccctatactgggacatcaagccttcacagaatcaagggcctctcctcccattgatgcccgacaatgtcatcctctgctacctatgtggctggagccatgggtcccttcatgtatcctccttggttggtggtttagtctctgggaggtctgggggatctggttgattgatattattgttcttcctatgagattgcaaaccccttcagctccttcggtcctttaactcctccactggggaccccgagctcagtccaatggttggctgtgagcatccaccagcagaggcctttttttttttttttaacaaagctgctttattatgttgcttagagcatgaccaggaaccagagcacagtccaagactgaagggaggaaaagggggggagtcaataaccccactgtttcatagtggtttgcaacccttttatatcacagcccactttaggcaaataatgaaaattatagtctccagggacagagaagatggtgcaggaagtgaagtgcctgctcagaaaatgggggcttgaatgtgagttcccagactctgtgtaagatgcccagcatcgaagtgcatgcttataacaccagcctggaggtagaagcttagaaacaggggtaccctgaagttgcttgttcaccagtgtccctgaatgggtaggtgcatgtttggtgagagaccctgtctcaaaaatcaaggtgtaggataattgaaaatacctagctttgagcttagatcatgcaaatgtgtacacacactcacacacaccacacacacaaaaaaatgcagagacagagagatacagagagacagagagatacagagacagagacagagagaaaaggagaaagtaaaaaacaaataatttaaagacccatggccacaaagaggctcaaagacaagcacgtataaaaccatacacatgtaattttaggagttttcagattccctggtacccgtgggtgatgcacaagctttgaatcccagtcttaaaatcttacgaagaacgtgttcgtgtgtgctaatttattgatgagaggaaaggaattgacaaagtgcccttccggagcttcctgcattacccagactcagggtttttttaaatgtacactcagaacagagtagctctgtgcaagggtagcaaccacgaagcttaataagaaacatatcgtgagagatctgcaaggcaaatctaggggctgaccaatctcacagtcacccactagcatgtcaacacaacttcccacctgtgctagccacttagcaattttgtgttgttctgttttgtttttgtttttaacaaagcaatttcaaagagatttctaattcatctaaacaaacaaaccaaaaggaaaacagcaaagacgccctgagcacttagcagagcagctatgcagttatgactcctgggtggagactttatatcaggcttcaactgaatacctagaacctactagtgctcttcatcaatccttgggaaggtcattttcttttggtgctgttttgagtttctatttgttaatgtcttcataattatacacgtgttgagcacagcatgcaaagtgattaggggaatctagttggagtggaatggatacccaaatattcagactttcttgtgactcttctttcttgtacccacatcaaaaaaaaaaaaaatggagatgagacatggtcagagtcactaaaaccagctgctacttttaattacgtggggagcagtttctaacattgccattattgaactgatgctgcctgggtggaaatggaaatcacttagtatttcttgttggcaaagaattactgaatggattaaatttccaaagggagaagtcagttacaagtcttttctttgtttattaggctttctgctatgataaattacactacttccagaagttacccttaggccatgggacactggactatcactctgctgtcacaagagattacagagttagtcaaggcagcttgtgacaccttcagggactgtcataaacttccagcaagtcattaatcctgaatgcaatactgtgtgtgtgtgtctatgtgtgtttgtatgtctgtgtgtgtcttatgtctgtgtctctgtgtgtgtgtgtgtttgtgtgtgtgtgtgtatgtatgcctgtgtgtgtcttatgtctgtgtttgtgtgtctgtgtgtgtcttatgtctgtgtttgtatgtctgtgtgtgtctgtgtgtgtcttatgtctgtgtctctgtgtgtgtgtgtgtgtatgtatgtatgtatgtatgtatgtgtatgtgtttgcatctctctgtgtgtctgcgcttatatatttgtgtatgtgtttatgtgttcgcctttgtgcgttgttggggattgaatccaggggaatacaaatgttaagaaagaacgttaccactaagcttcacctgtaggccttaaagcttttctttcttttaaaaattgtaattaattcattttcagtcaggatctccacacctcgtccctgctgctctagaactcactatttaaacacaatcgccctcaaacctgcagcaaccctcccgcctctaccctgcgagcactagaataataacaggtgaccccacacgcctagattaagacctttaaggtaaacattttactatattttagtctcataagacaagatgctacaataaagctgtacataaagttccctcgaatttcttgctattttaactcaaacataaggatttcctcctttttgattcaggtaacagaaaaaatacacaggtacatacatgtacacacatgaacacacacgcatcacaaccacatatgcgcacgcttgtgtgatctatcatttaccatgccactgaactcttctttccccataaattcctctggacttgtgtgccctccaggaagaccgcggagtctggagagctgcacgggctcaccacagatgagaagtttgtagaaggagtgtacagagtagaactggacaccaaatcgtactggaagacacttggcatttccccgttccatgaattcgcggatgtaagtggacacaccaagttgtttggattttgtttttagtctcaggaaattcccttcgctcttgctgtacgatgggcatgagtggaaagtagattccacagccagaatccacagtgctgggaaagcaagccttctgaatttttctaaaactcatttagcaacatggcctgaacctgttcacactgcttatggtcagctaactatatttatgtaaatattcatttctctgttgaggaaatgttagtatttgcttttgaggcaacctccagataccatggagggcatgtcatagtcaaagagagggctccctatggtatttctctaaattctggcatttcctttattccaaagcacatctagtgtccccagaagtttgggtagacaattcttggcaacacagagaattacaacatgttcaaaacccaacagcttaatatctaaatcatcaagcaaacatcacatggcaaagggatttctgaatcaaaactgtttcatccttatgatcaacctatggaggtctagcctcgacttacacccattttaccaataagctaagagaagctaagttcctcatcaaggacacaaggctagcatgtgtgagcaagtgacagagttgccctctatgttggttagtgtgccttagccagtgtctcagtaagaaatggagctaaatcaaaacccaaggccaacagccaaaggcacatgagtaacctttgcttggcactgggctcagtttccctggctcctctcagtcctcagttcacagaggcagctgtcatgcaaatagaatccaagcttgttggtcagacctggagataacaaattccatcaaaaatagctcctcatgtgacctagtttgctgtctgttgctatgatacacaccatgaccgaaaagcaaccctggggagagaagggtttatttcatcttacagcttacagttcaccatggaggaaagccaggtgggaacctggaagtggaaattgaagcagagaccagaaaggaatgctgtttactggctggcttagctccttttcttatacagcttaggtctatgtgcccaggggatggtactgccgagcataggctgagcccgcctacatcaaccattagtcaaaaaaaggtccatagacttgcctacaggccaatctcatggaggcaataccccagtggagggtccctcttcgcaggttactctagtttgtgtcaagttgacaaaacctaaccacaaagcacaaacagggtctgcccttgtggcttagccatggatgacactctcagatgatggtgttaccagacaaaccagaggggctcaccaagagtctgccacctaccaaggtagtactctactcctcactgggcaccaacacccatattagctgggccagtacaggacccttgctgtttcctgcatgaattgtccatagaccctgggtctcagcctgccgggagtacctgtaagtagtcgcctcaaacacattattcctgttggaagacttgtctgattctcttttagaactcaatcaacaaacgtttttattttgttttggctttttggagacaagatctctcataggccagcctgacttgaatgtagctgaggatgacctgtgctgctaatcttctcgcctcttcctcccaagtggtaggataataggcataagacaccacagcagttttactccataccagggctctgaacccagactttaaacactctatcaactgattcacattcccaccccatcattcaacaaacatttgaaaaataaaacccttctgccttgagcactctgctaaatacagcctttgagtgcggagtatttcctcacaaccagggtccaagatgaccccatcatacataccacggaaaattaggagatgtttttaggtctctttgcttggggtaatttttatgtgtgtgtgtacacagccctgtgcgtgtgtgtgtgtgtgtgtgtgtgtgtgtacaggcacacacgtgtatgcatgtagaggctacataaaaaccttaggtgtcattctcaggcactctgttcaccccttcacacagcccgaacacacaaaatttgaggcattagcctggagctcaccagttaggctagactgacttgccagcagaccccaggctgtctccatctccccagctctgggattacaaactctatcataccagacatttttatacatattctgagcataaaattcatgtcttcaggctaacaagtcaagagcttaaatgactgagctctcttacgtggtggattttttttaaaactacataatatcttttttttttttttcacttctggggaagaaacaaatgagcctgagtgacaatgcgacagaaaagaaattttgaggagtgtgtgtgtctgtgtgtgtggtggcacatgcctctcatctaatgctagaggctacagtagaatgctcctgaattagtggccagccaaggccaagggctagggttgtaactcagtggcagagggcttgcctagcattcgcaggatttgatccatagcgctataaataataataaataaatacaacagtctaagatgattctccctttcatttatctggatgttatttttgtgttagttttactctgtcatccaatcattgtttgccctatatttggacatttaaaaaaaatctttattccaagtgtgttcaaagctgtatccaaaacctgtccaccaaatgagtccaatgacatacatcttctatattaccatctgttccagatttggctgactcccggcacctgggctgttgctgcacccatgtctcagatagtctagtgatttgagaagtgactagtaattgcaaaatccagactttgtccagaaacttctatgagctccaaaactttcatttacatttctgccagccacaaaccgcttgtgttgtggagagaaccctgtgatgtcttcccacagcatctcagccttgtttcttcccttaaaatattcatcttttcacattagaacatgcaaagggacagtgggagcgaaacccctggactgggacgcacgaagccttcctttctggtcaggctctcactgtagaaacttaggccggtttcagcatgcagtctgctggagaatggctcctgccaacattccaggtctggaagtttgtagtggagttgttgataaccactgttcgccacaggtcttttgtttgtgggtgtcagtgtttctactctcctgacttttatctgaacccaagaaagggaacaatagccttcaagctctctgtgactctgatctgaccagggccacccacactgcagaaggaaacttgcaaagagagacctgcaattctctaagagctccacacagctccaaagacttaggcagcatattttaatctaattattcgtcccccaaccccaccccagaggacagttagacaataaaaggaagattaccagcttagcatcctgtgaacactttgtctgcagctcctacctctgggctctgttagaactagctgtctctcctctctcctaggtggttttcacagccaacgactctggccatcgccactacaccatcgcagccctgctcagcccatactcctacagcaccacggctgtcgtcagcaacccccagaattga

SEQ ID NO: 22 - DNA, 11842 bp, Artificial Sequence
  Synthetic
  misc_feature (1)..(97): Mouse Sequence
  misc_feature (98)..(7216): Human Sequence
  misc_feature (7217)..(11742): Cassette Sequence
  misc_feature (11743)..(11842): Mouse Sequence
agcttggcagggatcagcagcctgggttggaaggagggggtataaaagccccttcaccaggagaagccgtcacacagatccacaagctcctgacaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccatgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaatgagagagccttattttcctgtaaccttcagcaaatagcaaaagacacattccaagggcccacttctttactgtgggcactcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggcctagataacttcgtataatgtatgctatacgaagttatgctaggtaactataacggtcctaaggtagcgagctagcgagactcagcccaggaggaccaggatcttgccaaagcagtagcatcccatttgtaccaaaacagtgttcttgctctataaaccgtgttagcagctcagga

SEQ ID NO: 23 - DNA, 7394 bp, Artificial Sequence
  Synthetic
  misc_feature (1)..(97): Mouse Sequence
  misc_feature (98)..(7216): Human Sequence
  misc_feature (7217)..(7294): Cassette Sequence
  misc_feature (7295)..(7394): Mouse Sequence
agcttggcagggatcagcagcctgggttggaaggagggggtataaaagccccttcaccaggagaagccgtcacacagatccacaagctcctgacaggatggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccatgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtctcgagataacttcgtataatgtatgctatacgaagttatgctaggtaactataacggtcctaaggtagcgagctagcgagactcagcccaggaggaccaggatcttgccaaagcagtagcatcccatttgtaccaaaacagtgttcttgctctataaaccgtgttagcagctcagga

SEQ ID NO: 24 - DNA, 7119 bp, Artificial Sequence
  Synthetic
atggcttctcatcgtctgctcctcctctgccttgctggactggtatttgtgtctgaggctggccctacggtgagtgtttctgtgacatcccattcctacatttaagattcacgctaaatgaagtagaagtgactccttccagctttgccaaccagcttttattactagggcaagggtacccagcatctatttttaatataattaattcaaacttcaaaaagaatgaagttccactgagcttactgagctgggacttgaactctgagcattctacctcattgctttggtgcattaggtttgtaatatctggtacctctgtttcctcagatagatgatagaaataaagatatgatattaaggaagctgttaatactgaattttcagaaaagtatccctccataaaatgtatttgggggacaaactgcaggagattatattctggccctatagttattcaaaacgtatttattgattaatctttaaaaggcttagtgaacaatattctagtcagatatctaattcttaaatcctctagaagaattaactaatactataaaatgggtctggatgtagttctgacattattttataacaactggtaagagggagtgactatagcaacaactaaaatgatctcaggaaaacctgtttggccctatgtatggtacattacatcttttcagtaattccactcaaatggagacttttaacaaagcaactgttctcaggggacctattttctcccttaaaattcattatacacatccctggttgatagcagtgtgtctggaggcagaaaccattcttgctttggaaacaattacgtctgtgttatactgagtagggaagctcattaattgtcgacacttacgttcctgataatgggatcagtgtgtaattcttgtttcgctccagatttctaataccacaaagaataaatcctttcactctgatcaattttgttaacttctcacgtgtcttctctacacccagggcaccggtgaatccaagtgtcctctgatggtcaaagttctagatgctgtccgaggcagtcctgccatcaatgtggccatgcatgtgttcagaaaggctgctgatgacacctgggagccatttgcctctgggtaagttgccaaagaaccctcccacaggacttggttttatcttcccgtttgcccctcacttggtagagagaggctcacatcatctgctaaagaatttacaagtagattgaaaaacgtaggcagaggtcaagtatgccctctgaaggatgccctctttttgttttgcttagctaggaagtgaccaggaacctgagcatcatttaggggcagacagtagagaaaagaaggaatcagaactcctctcctctagctgtggtttgcaacccttttgggtcacagaacactttatgtaggtgatgaaaagtaaacattctatgcccagaaaaaatgcacagatacacacacatacaaaatcatatatgtgattttaggagtttcacagattccctggtgtccctgggtaacaccaaagctaagtgtccttgtcttagaattttaggaaaaggtataatgtgtattaacccattaacaaaaggaaaggaattcagaaatattattaaccaggcatctgtctgtagttaatatggatcacccaaaacccaaggcttttgcctaatgaacactttggggcacctactgtgtgcaaggctgggggctgtcaagctcagttaaaaaaaaaaagatagaagagatggatccatgaggcaaagtacagccccaggctaatcccacgatcacccgacttcatgtccaagagtggcttctcaccttcattagccagttcacaattttcatggagtttttctacctgcactagcaaaaacttcaaggaaaatacatattaataaatctaagcaaagtgaccagaagacagagcaatcaggagaccctttgcatccagcagaagaggaactgctaagtatttacatctccacagagaagaatttctgttgggttttaattgaaccccaagaaccacatgattcttcaaccattattgggaagatcattttcttaggtctggttttaactggctttttatttgggaattcatttatgtttatataaaatgccaagcataacatgaaaagtggttacaggactattctaagggagagacagaatggacaccaaaaatattccaatgttcttgtgaatcttttccttgcaccaggacaaaaaaaaaaagaagtgaaaagaagaaaggaggaggggcataatcagagtcagtaaagacaactgctatttttatctatcgtagctgttgcagtcaaatgggaagcaatttccaacattcaactatggagctggtacttacatggaaatagaagttgcctagtgtttgttgctggcaaagagttatcagagaggttaaatatataaaagggaaaagagtcagatacaggttcttcttcctactttaggttttccactgtgtgtgcaaatgatactccctggtggtgtgcagatgcctcaaagctatcctcacaccacaagggagaggagcgagatcctgctgtcctggagaagtgcagagttagaacagctgtggccacttgcatccaatcatcaatcttgaatcacagggactctttcttaagtaaacattatacctggccgggcacggtggctcacgcctgtaatcccagcactttgggatgccaaagtgggcatatcatctgaggtcaggagttcaagaccagcctggccaacatggcaaaactccgtctttatgaaaaatacaaaaattagccaggcatggtggcaggcgcctgtaatcccagctaattgggaggctgaggctggagaatcccttgaatctaggaggcagaggttgcagtgagctgagatcgtgccattgcactccagcctgggtgacaagagtaaaactctgtctcaaaaaaaaaaaattatacctacattctcttcttatcagagaaaaaaatctacagtgagcttttcaaaaagtttttacaaactttttgccatttaatttcagttaggagttttccctacttctgacttagttgaggggaaatgttcataacatgtttataacatgtttatgtgtgttagttggtgggggtgtattactttgccatgccatttgtttcctccatgcgtaacttaatccagactttcacaccttataggaaaaccagtgagtctggagagctgcatgggctcacaactgaggaggaatttgtagaagggatatacaaagtggaaatagacaccaaatcttactggaaggcacttggcatctccccattccatgagcatgcagaggtgagtatacagaccttcgagggttgttttggttttggtttttgcttttggcattccaggaaatgcacagttttactcagtgtaccacagaaatgtcctaaggaaggtgatgaatgaccaaaggttccctttcctattatacaagaaaaaattcacaacactctgagaagcaaatttctttttgactttgatgaaaatccacttagtaacatgacttgaacttacatgaaactactcatagtctattcattccactttatatgaatattgatgtatctgctgttgaaataatagtttatgaggcagccctccagaccccacgtagagtgtatgtaacaagagatgcaccattttatttctcgaaaacccgtaacattcttcattccaaaacacatctggcttctcggaggtctggacaagtgattcttggcaacacatacctatagagacaataaaatcaaagtaataatggcaacacaatagataacatttaccaagcatacaccatgtggcagacacaattataagtgttttccatatttaacctacttaatcctcaggaataagccactgaggtcagtcctattattatccccatcttatagatgaagaaaatgaggcaccaggaagtcaaataacttgtcaaaggtcacaagactaggaaatacacaagtagaaatgtttacaattaaggcccaggctgggtttgccctcagttctgctatgcctcgcattatgccccaggaaactttttcccttgtgaaagccaagcttaaaaaaagaaaagccacatttgtaacgtgctctgttcccctgcctatggtgaggatcttcaaacagttatacatggacccagtccccctgccttctccttaatttcttaagtcatttgaaacagatggctgtcatggaaatagaatccagacatgttggtcagagttaaagatcaactaattccatcaaaaatagctcggcatgaaagggaactattctctggcttagtcatggatgagactttcaattgctataaagtggttcctttattagacaatgttaccagggaaacaacaggggtttgtttgacttctggggcccacaagtcaacaagagagccccatctaccaaggagcatgtccctgactacccctcagccagcagcaagacatggaccccagtcagggcaggagcagggtttcggcggcgcccagcacaagacattgcccctagagtctcagcccctaccctcgagtaatagatctgcctacctgagactgttgtttgcccaagagctgggtctcagcctgatgggaaccatataaaaaggttcactgacatactgcccacatgttgttctctttcattagatcttagcttccttgtctgctcttcattcttgcagtattcattcaacaaacattaaaaaaaaaaaaaagcattctatgtgtggaacactctgctagatgctgtggatttagaaatgaaaatacatcccgacccttggaatggaagggaaaggactgaagtaagacagattaagcaggaccgtcagcccagcttgaagcccagataaatacggagaacaagagagagcgagtagtgagagatgagtcccaatgcctcactttggtgacgggtgcgtggtgggcttcatgcagcttcttctgataaatgcctccttcagaactggtcaactctaccttggccagtgacccaggtggtcatagtagatttaccaagggaaaatggaaacttttattaggagctcttaggcctcttcacttcatggatttttttttcctttttttttgagatggagttttgccctgtcacccaggctggaatgcagtggtgcaatctcagctcactgcaacctccgcctcccaggttcaagcaattctcctgcctcagcctcccgagtagctgggactacaggtgtgcgccaccacaccaggctaatttttgtattttttgtaaagacaggttttcaccacgttggccaggctggtctgaactccagacctcaggtgattcacctgtctcagcctcccaaagtgctgggattacaggtgtgagccaccgtgcccggctacttcatggatttttgattacagattatgcctcttacaatttttaagaagaatcaagtgggctgaaggtcaatgtcaccataagacaaaagacatttttattagttgattctagggaattggccttaaggggagccctttcttcctaagagattcttaggtgattctcacttcctcttgccccagtattatttttgtttttggtatggctcactcagatccttttttcctcctatccctaagtaatccgggtttctttttcccatatttagaacaaaatgtatttatgcagagtgtgtccaaacctcaacccaaggcctgtatacaaaataaatcaaattaaacacatctttactgtcttctacctctttcctgacctcaatatatcccaacttgcctcactctgagaaccaaggctgtcccagcacctgagtcgcagatattctactgatttgacagaactgtgtgactatctggaacagcattttgatccacaatttgcccagttacaaagcttaaatgagctctagtgcatgcatatatatttcaaaattccaccatgatcttccacactctgtattgtaaatagagccctgtaatgcttttacttcgtatttcattgcttgttatacataaaaatatacttttcttcttcatgttagaaaatgcaaagaataggagggtgggggaatctctgggcttggagacaggagacttgccttcctactatggttccatcagaatgtagactgggacaatacaataattcaagtctggtttgctcatctgtaaattgggaagaatgtttccagctccagaatgctaaatctctaagtctgtggttggcagccactattgcagcagctcttcaatgactcaatgcagttttgcattctccctaccttttttttctaaaaccaataaaatagatacagcctttaggctttctgggatttcccttagtcaagctagggtcatcctgactttcggcgtgaatttgcaaaacaagacctgactctgtactcctgctctaaggactgtgcatggttccaaaggcttagcttgccagcatatttgagctttttccttctgttcaaactgttccaaaatataaaagaataaaattaattaagttggcactggacttccggtggtcagtcatgtgtgtcatctgtcacgtttttcgggctctggtggaaatggatctgtctgtcttctctcataggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgt

SEQ ID NO: 25 - DNA, 24 bp, Artificial Sequence
  Synthetic
cacagacaatcagacgtaccagta

SEQ ID NO: 26 - DNA, 23 bp, Artificial Sequence
  Synthetic
gggacatctcggtttcctgactt

SEQ ID NO: 27 - DNA, 26 bp, Artificial Sequence
  Synthetic
tcatgtaatctggcttcagagtggga

SEQ ID NO: 28 - DNA, 20 bp, Artificial Sequence
  Synthetic
ccagctttgccagtttacga

SEQ ID NO: 29 - DNA, 22 bp, Artificial Sequence
  Synthetic
tccacactactgaactccacaa

SEQ ID NO: 30 - DNA, 27 bp, Artificial Sequence
  Synthetic
tgggaggcaattcttagtttcaatgga

SEQ ID NO: 31 - DNA, 18 bp, Artificial Sequence
  Synthetic
ttggacggttgccctctt

SEQ ID NO: 32 - DNA, 21 bp, Artificial Sequence
  Synthetic
cggaacactcgctctacgaaa

SEQ ID NO: 33 - DNA, 23 bp, Artificial Sequence
  Synthetic
tcccaaaggtgtctgtctgcaca

SEQ ID NO: 34 - DNA, 22 bp, Artificial Sequence
  Synthetic
gatggcttcccttcgactcttc

SEQ ID NO: 35 - DNA, 18 bp, Artificial Sequence
  Synthetic
gggccagcttcagacaca

SEQ ID NO: 36 - DNA, 22 bp, Artificial Sequence
  Synthetic
ctcctttgcctcgctggactgg

SEQ ID NO: 37 - DNA, 25 bp, Artificial Sequence
  Synthetic
cactgacatttctcttgtctcctct

SEQ ID NO: 38 - DNA, 22 bp, Artificial Sequence
  Synthetic
cccagggtgctggagaatccaa

SEQ ID NO: 39 - DNA, 19 bp, Artificial Sequence
  Synthetic
cggacagcatccaggactt

SEQ ID NO: 40 - DNA, 21 bp, Artificial Sequence
  Synthetic
gggctcaccacagatgagaag

SEQ ID NO: 41 - DNA, 22 bp, Artificial Sequence
  Synthetic
gccaagtgtcttccagtacgat

SEQ ID NO: 42 - DNA, 29 bp, Artificial Sequence
  Synthetic
agaaggagtgtacagagtagaactggaca

SEQ ID NO: 43 - DNA, 20 bp, Artificial Sequence
  Synthetic
cactgttcgccacaggtctt

SEQ ID NO: 44 - DNA, 21 bp, Artificial Sequence
  Synthetic
gttccctttcttgggttcaga

SEQ ID NO: 45 - DNA, 27 bp, Artificial Sequence
  Synthetic
tgtttgtgggtgtcagtgtttctactc

SEQ ID NO: 46 - DNA, 20 bp, Artificial Sequence
  Synthetic
gctcagcccatactcctaca

SEQ ID NO: 47 - DNA, 23 bp, Artificial Sequence
  Synthetic
gatgctactgctttggcaagatc

SEQ ID NO: 48 - DNA, 21 bp, Artificial Sequence
  Synthetic
caccacggctgtcgtcagcaa

SEQ ID NO: 49 - DNA, 18 bp, Artificial Sequence
  Synthetic
gcccaggaggaccaggat

SEQ ID NO: 50 - DNA, 20 bp, Artificial Sequence
  Synthetic
cctgagctgctaacacggtt

SEQ ID NO: 51 - DNA, 23 bp, Artificial Sequence
  Synthetic
cttgccaaagcagtagcatccca

SEQ ID NO: 52 - DNA, 20 bp, Artificial Sequence
  Synthetic
ggcaacttgcttgaggaaga

SEQ ID NO: 53 - DNA, 23 bp, Artificial Sequence
  Synthetic
agctacagaccatgcttagtgta

SEQ ID NO: 54 - DNA, 23 bp, Artificial Sequence
  Synthetic
aggtcagaaagcagagtggacca

SEQ ID NO: 55 - DNA, 20 bp, Artificial Sequence
  Synthetic
gcagcaacccagcttcactt

SEQ ID NO: 56 - DNA, 24 bp, Artificial Sequence
  Synthetic
tgccagtttaggaggaatatgttc

SEQ ID NO: 57 - DNA, 23 bp, Artificial Sequence
  Synthetic
cccaggcaattcctaccttccca

SEQ ID NO: 58 - DNA, 19 bp, Artificial Sequence
  Synthetic
actgagctgggacttgaac

SEQ ID NO: 59 - DNA, 25 bp, Artificial Sequence
  Synthetic
ctgaggaaacagaggtaccagatat

SEQ ID NO: 60 - DNA, 28 bp, Artificial Sequence
  Synthetic
tctgagcattctacctcattgctttggt

SEQ ID NO: 61 - DNA, 19 bp, Artificial Sequence
  Synthetic
tgcctcactctgagaacca

SEQ ID NO: 62 - DNA, 24 bp, Artificial Sequence
  Synthetic
agtcacacagttctgtcaaatcag

SEQ ID NO: 63 - DNA, 23 bp, Artificial Sequence
  Synthetic
aggctgtcccagcacctgagtcg

SEQ ID NO: 64 - DNA, 19 bp, Artificial Sequence
  Synthetic
cgcaacctccccttctacg

SEQ ID NO: 65 - DNA, 17 bp, Artificial Sequence
  Synthetic
gtccttcgggcacctcg

SEQ ID NO: 66 - DNA, 21 bp, Artificial Sequence
  Synthetic
cggctcggcttcaccgtcacc

SEQ ID NO: 67 - DNA, 18 bp, Artificial Sequence
  Synthetic
ggccgtgcatgtgttcag

SEQ ID NO: 68 - DNA, 20 bp, Artificial Sequence
  Synthetic
tcctgtgggagggttctttg

SEQ ID NO: 69 - DNA, 23 bp, Artificial Sequence
  Synthetic
aaggctgctgatgacacctggga

SEQ ID NO: 70 - DNA, 23 bp, Artificial Sequence
  Synthetic
ggttcccatttgctcttattcgt

SEQ ID NO: 71 - DNA, 20 bp, Artificial Sequence
  Synthetic
ccctctctctgagccctcta

SEQ ID NO: 72 - DNA, 27 bp, Artificial Sequence
  Synthetic
agattcagacacacacaacttaccagc

SEQ ID NO: 73 - DNA, 23 bp, Artificial Sequence
  Synthetic
cccacactgcagaaggaaacttg

SEQ ID NO: 74 - DNA, 21 bp, Artificial Sequence
  Synthetic
gctgcctaagtctttggagct

SEQ ID NO: 75 - DNA, 28 bp, Artificial Sequence
  Synthetic
agacctgcaattctctaagagctccaca

SEQ ID NO: 76 - DNA, 23 bp, Artificial Sequence
  Synthetic
ggttcccatttgctcttattcgt

SEQ ID NO: 77 - DNA, 20 bp, Artificial Sequence
  Synthetic
ccctctctctgagccctcta

SEQ ID NO: 78 - DNA, 27 bp, Artificial Sequence
  Synthetic
agattcagacacacacaacttaccagc

SEQ ID NO: 79 - DNA, 22 bp, Artificial Sequence
  Synthetic
ccagcttagcatcctgtgaaca

SEQ ID NO: 80 - DNA, 25 bp, Artificial Sequence
  Synthetic
gagaggagagacagctagttctaac

SEQ ID NO: 81 - DNA, 24 bp, Artificial Sequence
  Synthetic
ttgtctgcagctcctacctctggg

SEQ ID NO: 82 - DNA, 20 bp, Artificial Sequence
  Synthetic
ggcaacttgcttgaggaaga

SEQ ID NO: 83 - DNA, 23 bp, Artificial Sequence
  Synthetic
agctacagaccatgcttagtgta

SEQ ID NO: 84 - DNA, 23 bp, Artificial Sequence
  Synthetic
aggtcagaaagcagagtggacca

SEQ ID NO: 85 - DNA, 22 bp, Artificial Sequence
  Synthetic
tgtggagttcagtagtgtggag

SEQ ID NO: 86 - DNA, 22 bp, Artificial Sequence
  Synthetic
gccctcttcatacaggaatcac

SEQ ID NO: 87 - DNA, 29 bp, Artificial Sequence
  Synthetic
ttgacatgtgtgggtgagagattttactg

SEQ ID NO: 88 - DNA, 25 bp, Artificial Sequence
  Synthetic
cactgacatttctcttgtctcctct

SEQ ID NO: 89 - DNA, 19 bp, Artificial Sequence
  Synthetic
cggacagcatccaggactt

SEQ ID NO: 90 - DNA, 22 bp, Artificial Sequence
  Synthetic
cccagggtgctggagaatccaa

SEQ ID NO: 91 - DNA, 18 bp, Artificial Sequence
  Synthetic
ctgtccgaggcagtcctg

SEQ ID NO: 92 - DNA, 21 bp, Artificial Sequence
  Synthetic
gtgtcatcagcagcctttctg

SEQ ID NO: 93 - DNA, 16 bp, Artificial Sequence
  Synthetic
aatgtggccgtgcatg

SEQ ID NO: 94 - DNA, 18 bp, Artificial Sequence
  Synthetic
ctgtccgaggcagtcctg

SEQ ID NO: 95 - DNA, 21 bp, Artificial Sequence
  Synthetic
gtgtcatcagcagcctttctg

SEQ ID NO: 96 - DNA, 15 bp, Artificial Sequence
  Synthetic
atgtggccatgcatg

SEQ ID NO: 97 - protein, 1471 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSGRADALDDFDLDMLGSDALDDFDLDMLGSDALDDFDLDMLGSDALDDFDLDMLINCT

SEQ ID NO: 98 - protein, 1384 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 99 - protein, 62 aa, Artificial Sequence
  Synthetic
AAAGSGRADALDDFDLDMLGSDALDDFDLDMLGSDALDDFDLDMLGSDALDDFDLDMLINCT

SEQ ID NO: 100 - protein, 4 aa, Artificial Sequence
  Synthetic
GGGS

SEQ ID NO: 101 - protein, 5 aa, Artificial Sequence
  Synthetic
GGGGS

SEQ ID NO: 102 - protein, 473 aa, Artificial Sequence
  Synthetic
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 103 - protein, 130 aa, Artificial Sequence
  Synthetic
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 104 - protein, 181 aa, Artificial Sequence
  Synthetic
PSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 105 - protein, 124 aa, Artificial Sequence
  Synthetic
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 106 - RNA, 34 nt, Artificial Sequence
  Synthetic
ggccaacaugaggaucacccaugucugcagggcc

SEQ ID NO: 107 - protein, 18 aa, Artificial Sequence
  Synthetic
EGRGSLLTCGDVEENPGP

SEQ ID NO: 108 - protein, 19 aa, Artificial Sequence
  Synthetic
ATNFSLLKQAGDVEENPGP

SEQ ID NO: 109 - protein, 20 aa, Artificial Sequence
  Synthetic
QCTNYALLKLAGDVESNPGP

SEQ ID NO: 110 - protein, 22 aa, Artificial Sequence
  Synthetic
VKQTLNFDLLKLAGDVESNPGP

SEQ ID NO: 111 - DNA, 4152 bp, Artificial Sequence
  Synthetic
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 112 - DNA, 4414 bp, Artificial Sequence
  Synthetic
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacag

SEQ ID NO: 113 - DNA, 390 bp, Artificial Sequence
  Synthetic
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 114 - DNA, 1419 bp, Artificial Sequence
  Synthetic
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 115 - DNA, 187 bp, Artificial Sequence
  Synthetic
gcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacag

SEQ ID NO: 116 - DNA, 543 bp, Artificial Sequence
  Synthetic
ccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctct

SEQ ID NO: 117 - DNA, 372 bp, Artificial Sequence
  Synthetic
ggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 118 - DNA, 9043 bp, Artificial Sequence
  Synthetic
  misc_feature (1)..(34): First loxP site
  misc_feature (125)..(928): Neomycin phosphotransferase
  misc_feature (937)..(2190): Polyadenylation signal
  misc_feature (2218)..(2251): Second loxP site
  misc_feature (2306)..(6457): Codon-optimized dCas9
  misc_feature (2309)..(2356): NLS
  misc_feature (6512)..(6532): NLS
  misc_feature (6533)..(6719): VP64
  misc_feature (6728 )..(6781): T2A
  misc_feature (6782)..(7171): MCP
  misc_feature (7226)..(7246): NLS
  misc_feature (7262)..(7804): p65
  misc_feature (7829)..(8200): HSF1
  misc_feature (8224)..(8820): Woodchuck hepatitis virus posttranscriptional regulatory element (WPRE)
ataacttcgtataatgtatgctatacgaagttattaggtccctcgacctgcaggaattgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagttattaggtccctcgacctgcagcccaagctagtgcccgggaattcgctagggccaccatgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacaggcagtggagagggcagaggaagtctgctaacatgcggtgacgtcgaggagaatcctggcccaatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatggcaggcatg

SEQ ID NO: 119 - DNA, 3812 bp, Artificial Sequence
  Synthetic
  misc_feature (1)..(32): First rox site
  misc_feature (111)..(710): Puromycin-N-acetyltransferase
  misc_feature (797)..(2338): Polyadenylation signal
  misc_feature (2363)..(2394): Second rox site
  misc_feature (2401)..(2640): First U6 promoter
  misc_feature (2641)..(2797): First guide RNA coding sequence
  misc_feature (2883)..(3122): Second U6 promoter
  misc_feature (3123)..(3279): Second guide RNA coding sequence
  misc_feature (3364)..(3603): Third U6 promoter
  misc_feature (3604)..(3760): Third guide RNA coding sequence
taactttaaataatgccaattatttaaagttacctgcaggacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcataacttcgtataatgtatgctatacgggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagccgacgctaggtcgtcagtcaaagtacgtacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttagatgttttactagccagatttttcctcctctcctgactactcccagtcatagctgtccctcttctcttatggagatccctcgaggacatgaggtcgtcgctgtaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgacactgggtcgtgatcgggtacctaactttaaataatgccaattatttaaagttagctagctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 120 - DNA, 3814 bp, Artificial Sequence
  Synthetic
  misc_feature (1)..(32): First rox site
  misc_feature (111)..(710): Puromycin-N-acetyltransferase
  misc_feature (797)..(2338): Polyadenylation signal
  misc_feature (2363)..(2394): Second rox site
  misc_feature (2401)..(2640): First U6 promoter
  misc_feature (2642)..(2798): First Ttr guide RNA coding sequence
  misc_feature (2884)..(3123): Second U6 promoter
  misc_feature (3125)..(3281): Second Ttr guide RNA coding sequence
  misc_feature (3366)..(3605): Third U6 promoter
  misc_feature (3606)..(3762): Third Ttr guide RNA coding sequence
taactttaaataatgccaattatttaaagttacctgcaggacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgatgatctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcataacttcgtataatgtatgctatacgggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagccgacgctaggtcgtcagtcaaagtacgtacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttagatgttttactagccagatttttcctcctctcctgactactcccagtcatagctgtccctcttctcttatggagatccctcgaggacatgaggtcgtcgctgtaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtcgacactgggtcgtgatcgggtacctaactttaaataatgccaattatttaaagttagctagctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 121 - DNA, 20 bp, Artificial Sequence
  Synthetic
acggttgccctctttcccaa

SEQ ID NO: 122 - DNA, 20 bp, Artificial Sequence
  Synthetic
actgtcagactcaaaggtgc

SEQ ID NO: 123 - DNA, 20 bp, Artificial Sequence
  Synthetic
gacaataagtagtcttactc

SEQ ID NO: 124 - RNA, 157 nt, Artificial Sequence
  Synthetic
acgguugcccucuuucccaaguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 125 - RNA, 157 nt, Artificial Sequence
  Synthetic
acugucagacucaaaggugcguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 126 - RNA, 157 nt, Artificial Sequence
  Synthetic
gacaauaaguagucuuacucguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 127 - RNA, 137 nt, Artificial Sequence
  Synthetic
guuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 128 - RNA, 20 nt, Artificial Sequence
  Synthetic
acgguugcccucuuucccaa

SEQ ID NO: 129 - RNA, 20 nt, Artificial Sequence
  Synthetic
acugucagacucaaaggugc

SEQ ID NO: 130 - RNA, 20 nt, Artificial Sequence
  Synthetic
gacaauaaguagucuuacuc

SEQ ID NO: 131 - protein, 1965 aa, Artificial Sequence
  Synthetic
MKRPAATKKAGQAKKKKDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSAGGGGSGGGGSGGGGSGPKKKRKVAAAGSGRADALDDFDLDMLGSDALDDFDLDMLGSDALDDFDLDMLGSDALDDFDLDMLINCTGSGEGRGSLLTCGDVEENPGPMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVAAAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 132 - RNA, 157 nt, Artificial Sequence
  Synthetic
  misc_feature (1)..(20): n is a, c, g, or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcu

SEQ ID NO: 133 - DNA, 5898 bp, Artificial Sequence
  Synthetic
  misc_feature (1)..(4152): dCas9
  misc_feature (4228)..(4414): VP64
  misc_feature (4423)..(4476): T2A
  misc_feature (4477)..(4866): MCP
  misc_feature (4957)..(5499): P65
  misc_feature (5524)..(5895): HSF1
atgaaaaggccggcggccacgaaaaaggccggccaggcaaaaaagaaaaaggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagcgctggaggaggtggaagcggaggaggaggaagcggaggaggaggtagcggacctaagaaaaagaggaaggtggcggccgctggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactgtacaggcagtggagagggcagaggaagtctgctaacatgcggtgacgtcgaggagaatcctggcccaatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctga

SEQ ID NO: 134 - DNA, 1412 bp, Artificial Sequence
  Synthetic
  misc_feature (1)..(240): hU6 promoter
  misc_feature (241)..(260): Guide
  misc_feature (261)..(397): SAM Tracr
  misc_feature (398)..(449): Extended PolIII term
  misc_feature (483)..(722): hU6 promoter
  misc_feature (723)..(742): Guide
  misc_feature (743)..(879): SAM Tracr
  misc_feature (880)..(931): Extended PolIII term
  misc_feature (964)..(1203): hU6 promoter
  misc_feature (1204)..(1223): Guide
  misc_feature (1224)..(1360): SAM Tracr
  misc_feature (1361)..(1412): Extended PolIII term
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccnnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 135 - DNA, 1414 bp, Artificial Sequence
  Synthetic
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgtttt

SEQ ID NO: 136 - DNA, 1490 bp, Artificial Sequence
  Synthetic
  misc_feature (28)..(267): hU6 promoter
  misc_feature (269)..(288): Guide1
  misc_feature (289)..(425): SAM Tracr
  misc_feature (426)..(477): Extended terminator
  misc_feature (511)..(750): hU6 promoter
  misc_feature (752)..(771): Guide2
  misc_feature (772)..(908): SAM Tracr
  misc_feature (909)..(960): Extended terminator
  misc_feature (993)..(1232): hU6 promoter
  misc_feature (1233)..(1252): Guide3
  misc_feature (1253)..(1389): SAM Tracr
  misc_feature (1390)..(1441): Extended terminator
gctagccataagactcggccttagaactttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttgagctccataagactcggccttagaacaagctttttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttatgcatgtggctcccatttatacctggccggctttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgttttagagctagaaatagcaagttaaaataaggctagtccgttttggtcacccagtgaggaagctaggacagacctaggacggttgcctgcagg

SEQ ID NO: 137 - DNA, 490 bp, Artificial Sequence
  Synthetic
  misc_feature (1)..(318): mU6 promoter
  misc_feature (320)..(339): Guide1
  misc_feature (340)..(476): SAM Tracr
  misc_feature (477)..(482): Small terminator
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacggttgccctctttcccaagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttctagaggc

SEQ ID NO: 138 - DNA, 490 bp, Artificial Sequence
  Synthetic
  misc_feature (1)..(318): mU6 promoter
  misc_feature (320)..(339): Guide2
  misc_feature (340)..(476): SAM Tracr
  misc_feature (477)..(482): Small terminator
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgactgtcagactcaaaggtgcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttctagaggc

SEQ ID NO: 139 - DNA, 489 bp, Artificial Sequence
  Synthetic
  misc_feature (1)..(318): mU6 promoter
  misc_feature (319)..(338): Guide3
  misc_feature (339)..(475): SAM Tracr
  misc_feature (476)..(481): Small terminator
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacaataagtagtcttactcgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttctagaggc

SEQ ID NO: 140 - RNA, 139 nt, Artificial Sequence
  Synthetic
guuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuu

SEQ ID NO: 141 - RNA, 159 nt, Artificial Sequence
  Synthetic
  misc_feature (1)..(20): n is a, c, g, or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuu

SEQ ID NO: 142 - RNA, 16 nt, Artificial Sequence
  Synthetic
guuuuagagcuaugcu

SEQ ID NO: 143 - RNA, 67 nt, Artificial Sequence
  Synthetic
agcauagcaaguuaaaauaaggcuaguccguuaucaacuugaaaaaguggcaccgagucggugcuuu

SEQ ID NO: 144 - RNA, 72 nt, Artificial Sequence
  Synthetic
aaacagcauagcaaguuaaaauaaggcuaguccguuaucaacuugaaaaaguggcaccgagucggugcuuuu

SEQ ID NO: 145 - RNA, 82 nt, Artificial Sequence
  Synthetic
guuggaaccauucaaaacagcauagcaaguuaaaauaaggcuaguccguuaucaacuugaaaaaguggcaccgagucggugc

SEQ ID NO: 146 - RNA, 77 nt, Artificial Sequence
  Synthetic
guuuuagagcuagaaauagcaaguuaaaauaaggcuaguccguuaucaacuugaaaaaguggcaccgagucggugcu

SEQ ID NO: 147 - RNA, 82 nt, Artificial Sequence; identical to SEQ ID NO: 145
  Synthetic

SEQ ID NO: 148 - RNA, 76 nt, Artificial Sequence
  Synthetic
guuuuagagcuagaaauagcaaguuaaaauaaggcuaguccguuaucaacuugaaaaaguggcaccgagucggugc

SEQ ID NO: 149 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 150 - RNA, 83 nt, Artificial Sequence
  Synthetic
guuuuagagcuagaaauagcaaguuaaaauaaggcuaguccguuaucaacuugaaaaaguggcaccgagucggugcuuuuuuu

SEQ ID NO: 151 - RNA, 80 nt, Artificial Sequence
  Synthetic
guuuuagagcuagaaauagcaaguuaaaauaaggcuaguccguuaucaacuugaaaaaguggcaccgagucggugcuuuu

SEQ ID NO: 152 - RNA, 92 nt, Artificial Sequence
  Synthetic
guuuaagagcuaugcuggaaacagcauagcaaguuuaaauaaggcuaguccguuaucaacuugaaaaaguggcaccgagucggugcuuuuuu

SEQ ID NO: 153 - DNA, 23 bp, Artificial Sequence
  Synthetic
  misc_feature (2)..(21): n is a, c, g, or t
gnnnnnnnnnnnnnnnnnnnngg

SEQ ID NO: 154 - DNA, 23 bp, Artificial Sequence
  Synthetic
  misc_feature (1)..(21): n is a, c, g, or t
nnnnnnnnnnnnnnnnnnnnngg

SEQ ID NO: 155 - DNA, 25 bp, Artificial Sequence
  Synthetic
  misc_feature (3)..(23): n is a, c, g, or t
ggnnnnnnnnnnnnnnnnnnnnngg

(19) United States
(12) Patent Application Publication
(10) Pub. No.: US 2023/0102342 A1
(43) Pub. Date: Mar. 30, 2023

(54) NON-HUMAN ANIMALS COMPRISING A HUMANIZED TTR LOCUS COMPRISING A V30M MUTATION AND METHODS OF USE

(71) Applicant: Regeneron Pharmaceuticals, Inc., Tarrytown, NY (US)

(72) Inventors: Meghan Drummond Samuelson, Katonah, NY (US); Jeffery Haines, New York, NY (US); Charleen Hunt, Montvale, NJ (US); Guochun Gong, Pleasantville, NY (US); Brian Zambrowicz, Sleepy Hollow, NY (US)

(73) Assignee: Regeneron Pharmaceuticals, Inc., Tarrytown, NY (US)

(21) Appl. No.: 17/759,539

(22) PCT Filed: Mar. 23, 2021
(86) PCT No.: PCT/US2021/023674

§ 371 Date: Jul. 27, 2022

Related U.S. Application Data
(60) Provisional application No. 62/993,289, filed on Mar. 23, 2020.

(51) Int. Cl. A01K 67/027; C07K 14/47

(52) U.S. Cl. CPC A01K 67/0278; C07K 14/47; A01K 2217/072; A01K 2227/105; A01K 2267/0318

(57) ABSTRACT

Non-human animal genomes, non-human animal cells, and non-human animals comprising a humanized TTR locus comprising a V30M mutation and methods of making and using such non-human animal genomes, non-human animal cells, and non-human animals are provided. Non-human animal cells or non-human animals comprising a humanized TTR locus express a human TTR protein or a chimeric TTR protein, fragments of which are from human TTR. Methods are provided for using such non-human animals comprising a humanized TTR locus to assess in vivo efficacy of human-TTR-targeting reagents such as nuclease agents designed to target human TTR.

### CROSS REFERENCE TO RELATED APPLICATIONS

This application claims the benefit of U.S. Application No. 62/993,289, filed Mar. 23, 2020, which is herein incorporated by reference in its entirety for all purposes.

### REFERENCE TO A SEQUENCE LISTING SUBMITTED AS A TEXT FILE VIA EFS WEB

The Sequence Listing written in file 556520SEQLIST.txt is 211 kilobytes, was created on Mar. 14, 2021, and is hereby incorporated by reference.

### BACKGROUND

Transthyretin (TTR) is a protein found in the serum and cerebrospinal fluid that carries thyroid hormone and retinol-binding protein to retinol. The liver secretes TTR into the blood, while the choroid plexus secretes it into the cerebrospinal fluid. TTR is also produced in the retinal pigmented epithelium and secreted into the vitreous. Misfolded and aggregated TTR accumulates in multiple tissues and organs in the amyloid diseases senile systemic amyloidosis (SSA), familial amyloid polyneuropathy (FAP), and familial amyloid cardiomyopathy (FAC).

There remains a need for suitable non-human animals providing the true human target or a close approximation of the true human target of human-TTR-targeting reagents at the endogenous TTR locus, thereby enabling testing of the efficacy and mode of action of such agents in live animals as well as pharmacokinetic and pharmacodynamics studies in a setting where the humanized protein and humanized gene are the only version of TTR present.

### SUMMARY

Non-human animals, non-human animal cells, and non-human animal genomes comprising a humanized TTR locus comprising a V30M mutation are provided, as well as methods of making and using such non-human animals, non-human animal cells, and non-human animal genomes. Also provided are humanized non-human animal TTR genes comprising a V30M mutation, nuclease agents and/or targeting vectors for use in humanizing a non-human animal TTR gene, and methods of making and using such humanized TTR genes.

In one aspect, provided are non-human animals, non-human animal cells, and non-human animal genomes comprising in their genome a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous TTR locus comprises a V30M mutation. In one aspect, provided are non-human animals, non-human animal cells, and non-human animal genomes comprising in their genome a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous TTR locus comprises a V30M mutation, and wherein a humanized TTR protein (e.g., transthyretin precursor protein or mature transthyretin protein) is expressed from the humanized endogenous TTR locus.

In some such non-human animals, non-human animal cells, and non-human animal genomes, the human TTR sequence comprises the V30M mutation. In some such non-human animals, non-human animal cells, and non-human animal genomes, the humanized endogenous TTR locus comprises an endogenous TTR promoter, wherein the human TTR sequence is operably linked to the endogenous TTR promoter. In some such non-human animals, non-human animal cells, and non-human animal genomes, at least one intron and at least one exon of the endogenous TTR locus have been deleted and replaced with the corresponding human TTR sequence.

In some such non-human animals, non-human animal cells, and non-human animal genomes, the humanized endogenous TTR locus comprises a human TTR 3′ untranslated region. In some such non-human animals, non-human animal cells, and non-human animal genomes, the humanized endogenous TTR locus comprises an endogenous TTR 3′ untranslated region. In some such non-human animals, non-human animal cells, and non-human animal genomes, the humanized endogenous TTR locus comprises a human TTR 3′ untranslated region and an endogenous TTR 3′ untranslated region. In some such non-human animals, non-human animal cells, and non-human animal genomes, the endogenous TTR 5′ untranslated region has not been deleted and replaced with the corresponding human TTR sequence.

In some such non-human animals, non-human animal cells, and non-human animal genomes, the humanized endogenous TTR locus encodes a transthyretin precursor protein comprising a human mature transthyretin protein sequence. Optionally, the human mature transthyretin protein sequence comprises the sequence set forth in SEQ ID NO: 5, and optionally the human mature transthyretin protein sequence is encoded by a sequence comprising the sequence set forth in SEQ ID NO: 10.

In some such non-human animals, non-human animal cells, and non-human animal genomes, the humanized endogenous TTR locus encodes a transthyretin precursor protein comprising a human transthyretin signal peptide sequence. Optionally, the human transthyretin signal peptide sequence comprises the sequence set forth in SEQ ID NO: 3, and optionally the human transthyretin signal peptide sequence is encoded by a sequence comprising the sequence set forth in SEQ ID NO: 8.

In some such non-human animals, non-human animal cells, and non-human animal genomes, the entire TTR coding sequence of the endogenous TTR locus has been deleted and replaced with the corresponding human TTR sequence. Optionally, a region of the endogenous TTR locus from the TTR start codon to the TTR stop codon has been deleted and replaced with the corresponding human TTR sequence.

In some such non-human animals, non-human animal cells, and non-human animal genomes, a region of the endogenous TTR locus from the TTR start codon to the TTR stop codon has been deleted and replaced with a human TTR sequence comprising the corresponding human TTR sequence and a human TTR 3′ untranslated region, the endogenous TTR 5′ untranslated region has not been deleted and replaced with the human TTR sequence, and the humanized endogenous TTR locus comprises an endogenous TTR promoter, wherein the human TTR sequence is operably linked to the endogenous TTR promoter. In some such non-human animals, non-human animal cells, and non-human animal genomes, a region of the endogenous TTR locus from the TTR start codon to the TTR stop codon has been deleted and replaced with a human TTR sequence comprising the corresponding human TTR sequence and a human TTR 3′ untranslated region, the endogenous TTR 5′ and 3′ untranslated regions have not been deleted and replaced with the human TTR sequence, and the humanized endogenous TTR locus comprises an endogenous TTR promoter, wherein the human TTR sequence is operably linked to the endogenous TTR promoter.

In some such non-human animals, non-human animal cells, and non-human animal genomes, (i) the human TTR sequence at the humanized endogenous TTR locus comprises a sequence at least 90%, at least 95%, at least 96%, at least 97%, at least 98%, at least 99%, or 100% identical to the sequence set forth in SEQ ID NO: 24; and/or (ii) the humanized endogenous TTR locus encodes a transthyretin precursor protein comprising a sequence at least 90%, at least 95%, at least 96%, at least 97%, at least 98%, at least 99%, or 100% identical to the sequence set forth in SEQ ID NO: 2 or encodes a mature transthyretin protein comprising a sequence at least 90%, at least 95%, at least 96%, at least 97%, at least 98%, at least 99%, or 100% identical to the sequence set forth in SEQ ID NO: 5; and/or (iii) the humanized endogenous TTR locus comprises a transthyretin precursor protein coding sequence comprising a sequence at least 90%, at least 95%, at least 96%, at least 97%, at least 98%, at least 99%, or 100% identical to the sequence set forth in SEQ ID NO: 7 or comprises a mature transthyretin protein coding sequence comprising a sequence at least 90%, at least 95%, at least 96%, at least 97%, at least 98%, at least 99%, or 100% identical to the sequence set forth in SEQ ID NO: 10; and/or (iv) the humanized endogenous TTR locus comprises a sequence at least 90%, at least 95%, at least 96%, at least 97%, at least 98%, at least 99%, or 100% identical to the sequence set forth in SEQ ID NO: 22 or 23.

In some such non-human animals, non-human animal cells, and non-human animal genomes, the humanized endogenous TTR locus encodes a transthyretin precursor protein comprising an endogenous transthyretin signal peptide sequence. Optionally, the endogenous transthyretin signal peptide sequence comprises the sequence set forth in SEQ ID NO: 14, and optionally the endogenous transthyretin signal peptide sequence is encoded by a sequence comprising the sequence set forth in SEQ ID NO: 17.

In some such non-human animals, non-human animal cells, and non-human animal genomes, the first exon of the endogenous TTR locus has not been deleted and replaced with the corresponding human TTR sequence. Optionally, the first exon and first intron of the endogenous TTR locus have not been deleted and replaced with the corresponding human TTR sequence.

In some such non-human animals, non-human animal cells, and non-human animal genomes, a region of the endogenous TTR locus from the start of the second TTR exon to the TTR stop codon has been deleted and replaced with the corresponding human TTR sequence.

In some such non-human animals, non-human animal cells, and non-human animal genomes, a region of the endogenous TTR locus from the second TTR exon to the TTR stop codon has been deleted and replaced with a human TTR sequence comprising the corresponding human TTR sequence and a human TTR 3′ untranslated region, the endogenous TTR 5′ untranslated region has not been deleted and replaced with the corresponding human TTR sequence, and the humanized endogenous TTR locus comprises an endogenous TTR promoter, wherein the human TTR sequence is operably linked to the endogenous TTR promoter. In some such non-human animals, non-human animal cells, and non-human animal genomes, a region of the endogenous TTR locus from the second TTR exon to the TTR stop codon has been deleted and replaced with a human TTR sequence comprising the corresponding human TTR sequence and a human TTR 3′ untranslated region, the endogenous TTR 5′ and 3′ untranslated regions have not been deleted and replaced with the corresponding human TTR sequence, and the humanized endogenous TTR locus comprises an endogenous TTR promoter, wherein the human TTR sequence is operably linked to the endogenous TTR promoter.

In some such non-human animals, non-human animal cells, and non-human animal genomes, the humanized endogenous TTR locus does not comprise a selection cassette or a reporter gene. In some such non-human animals, non-human animal cells, and non-human animal genomes, the non-human animal is homozygous for the humanized endogenous TTR locus. In some such non-human animals, non-human animal cells, and non-human animal genomes, the non-human animal comprises the humanized endogenous TTR locus in its germline.

In some such non-human animals, non-human animal cells, and non-human animal genomes, the non-human animal is a mammal. Optionally, the non-human animal is a rodent. Optionally, the non-human animal is a rat or mouse. Optionally, the non-human animal is a mouse.

In some such non-human animals, serum levels of transthyretin protein expressed from the humanized endogenous TTR in the non-human animal are at least about 20 μg/mL (e.g., at least 20 μg/mL).

In some such non-human animals or non-human animal cells, the non-human animal or non-human animal cell. has been seeded with exogenous, pre-formed transthyretin aggregates or fibrils. Optionally, the exogenous, pre-formed transthyretin aggregates or fibrils comprise a V30M mutation. Optionally, the exogenous, pre-formed transthyretin aggregates or fibrils are human. Optionally, the exogenous, pre-formed transthyretin aggregates or fibrils are in the liver, the lung, the heart, the spleen, the kidney, or any combination thereof of the non-human animal. Optionally, the exogenous, pre-formed transthyretin aggregates or fibrils are in the liver of the non-human animal.

Some such non-human animals, non-human animal cells, or non-human animal genomes further comprise in their genome a genomically integrated expression cassette, wherein the expression cassette comprises: (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; and (b) a nucleic acid encoding a chimeric adaptor protein comprising an adaptor protein fused to one or more transcriptional activation domains. Some such non-human animals, non-human animal cells, or non-human animal genomes further comprise one or more guide RNAs or an expression cassette that encodes the one or more guide RNAs, each guide RNA comprising one or more adaptor-binding elements to which the chimeric adaptor protein can specifically bind, wherein each of the one or more guide RNAs is capable of forming a complex with the Cas protein and guiding it to a target sequence within a target gene, and wherein at least one of the one or more guide RNAs targets the humanized endogenous TTR locus. Some such non-human animals, non-human animal cells, or non-human animal genomes further comprise a second genomically integrated expression cassette that encodes one or more guide RNAs each comprising one or more adaptor-binding elements to which the chimeric adaptor protein can specifically bind, wherein each of the one or more guide RNAs is capable of forming a complex with the Cas protein and guiding it to a target sequence within a target gene, and wherein at least one of the one or more guide RNAs targets the humanized endogenous TTR locus. In some such non-human animals, non-human animal cells, or non-human animal genomes, the first expression cassette is integrated into a Rosa26 locus, the Cas protein is a Cas9 protein comprising mutations corresponding to D10A and N863A when optimally aligned with a Streptococcus pyogenes Cas9 protein, the one or more transcriptional activator domains in the chimeric Cas protein comprise VP64, the adaptor protein comprises an MS2 coat protein or a functional fragment or variant thereof, the one or more transcriptional activation domains in the chimeric adaptor protein comprise p65 and HSF1, the non-human animal further comprises one or more guide RNAs or an expression cassette that encodes the one or more guide RNAs, each of the one or more guide RNAs comprises two adaptor-binding elements to which the chimeric adaptor protein can specifically bind, the two adaptor-binding elements comprise a first adaptor-binding element within a first loop of each of the one or more guide RNAs and a second adaptor-binding element within a second loop of each of the one or more guide RNAs, and the target sequence is within a region 200 base pairs upstream of the transcription start site and 1 base pair downstream of the transcription start site.

Some such non-human animals, non-human animal cells, or non-human animal genomes further comprise one or more guide RNAs or an expression cassette that encodes the one or more guide RNAs, each guide RNA comprising one or more adaptor-binding elements to which the chimeric adaptor protein can specifically bind, and wherein each of the one or more guide RNAs is capable of forming a complex with the Cas protein and guiding it to a target sequence within a target gene. Some such non-human animals, non-human animal cells, or non-human animal genomes further comprise a second genomically integrated expression cassette that encodes one or more guide RNAs each comprising one or more adaptor-binding elements to which the chimeric adaptor protein can specifically bind, and wherein each of the one or more guide RNAs is capable of forming a complex with the Cas protein and guiding it to a target sequence within a target gene.

In some such non-human animals, non-human animal cells, or non-human animal genomes, the target sequence comprises a regulatory sequence within the target gene. Optionally, the regulatory sequence comprises a promoter or an enhancer. In some such non-human animals, non-human animal cells, or non-human animal genomes, the target sequence is within 200 base pairs of the transcription start site of the target gene. Optionally, the target sequence is within a region 200 base pairs upstream of the transcription start site and 1 base pair downstream of the transcription start site.

In some such non-human animals, non-human animal cells, or non-human animal genomes, the sequence encoding each of the one or more guide RNAs is operably linked to a different U6 promoter. In some such non-human animals, non-human animal cells, or non-human animal genomes, each of the one or guide RNAs comprises two adaptor-binding elements to which the chimeric adaptor protein can specifically bind. Optionally, a first adaptor-binding element is within a first loop of each of the one or more guide RNAs, and a second adaptor-binding element is within a second loop of each of the one or more guide RNAs. Optionally, each of one or more guide RNAs is a single guide RNA comprising a CRISPR RNA (crRNA) portion fused to a transactivating CRISPR RNA (tracrRNA) portion, and wherein the first loop is the tetraloop corresponding to residues 13-16 of SEQ ID NO: 146, 148, 150, or 151, and the second loop is the stem loop 2 corresponding to residues 53-56 of SEQ ID NO: 146, 148, 150, or 151.

In some such non-human animals, non-human animal cells, or non-human animal genomes, the adaptor-binding element comprises the sequence set forth in SEQ ID NO: 106. Optionally, each of the one or more guide RNAs comprises the sequence set forth in SEQ ID NO: 127, 132, 140, or 141.

In some such non-human animals, non-human animal cells, or non-human animal genomes, at least one of the one or more guide RNAs targets the humanized endogenous TTR locus. Optionally, the Ttr-targeting guide RNA targets a sequence comprising the sequence set forth in any one of SEQ ID NOS: 121-123 or optionally wherein the Ttr-targeting guide RNA comprises the sequence set forth in any one of SEQ ID NOS: 124-126.

In some such non-human animals, non-human animal cells, or non-human animal genomes, the one or more guide RNAs target two or more target genes. In some such non-human animals, non-human animal cells, or non-human animal genomes, the one or more guide RNAs comprise multiple guide RNAs that target a single target gene. In some such non-human animals, non-human animal cells, or non-human animal genomes, the one or more guide RNAs comprise at least three guide RNAs that target a single target gene. Optionally, the at least three guide RNAs target the humanized endogenous TTR locus, and wherein a first guide RNA targets a sequence comprising SEQ ID NO: 121 or comprises the sequence set forth in SEQ ID NO: 124, a second guide RNA targets a sequence comprising SEQ ID NO: 122 or comprises the sequence set forth in SEQ ID NO: 125, and a third guide RNA targets a sequence comprising SEQ ID NO: 123 or comprises the sequence set forth in SEQ ID NO: 126.

In some such non-human animals, non-human animal cells, or non-human animal genomes, the Cas protein is a Cas9 protein. In some such non-human animals, non-human animal cells, or non-human animal genomes, the Cas9 protein is a Streptococcus pyogenes Cas9 protein. Optionally, Cas9 protein comprises mutations corresponding to D10A and N863A when optimally aligned with a Streptococcus pyogenes Cas9 protein. In some such non-human animals, non-human animal cells, or non-human animal genomes, the sequence encoding the Cas protein is codon-optimized for expression in the non-human animal.

In some such non-human animals, non-human animal cells, or non-human animal genomes, the one or more transcriptional activator domains in the chimeric Cas protein are selected from: VP16, VP64, p65, MyoD1, HSF1, RTA, SET7/9, and a combination thereof. Optionally, the one or more transcriptional activator domains in the chimeric Cas protein comprise VP64. Optionally, the chimeric Cas protein comprises from N-terminus to C-terminus: the catalytically inactive Cas protein; a nuclear localization signal; and the VP64 transcriptional activator domain. Optionally, the chimeric Cas protein comprises a sequence at least 90%, at least 95%, at least 96%, at least 97%, at least 98%, at least 99%, or 100% identical to the sequence set forth in SEQ ID NO: 97. Optionally, the segment of the first expression cassette encoding the chimeric Cas protein comprises a sequence at least 90%, at least 95%, at least 96%, at least 97%, at least 98%, at least 99%, or 100% identical to the sequence set forth in SEQ ID NO: 112.

In some such non-human animals, non-human animal cells, or non-human animal genomes, the first expression cassette further comprises a polyadenylation signal or transcription terminator upstream of the segment encoding the chimeric Cas protein, the polyadenylation signal or transcription terminator is flanked by recombinase recognition sites, and the polyadenylation signal or transcription terminator has been excised in a tissue-specific manner. Optionally, the polyadenylation signal or transcription terminator has been excised in the liver. Optionally, the recombinase is a Cre recombinase. Optionally, the non-human animal, non-human animal cell, or non-human animal genome further comprises a genomically integrated recombinase expression cassette comprising a recombinase coding sequence operably linked to a tissue-specific promoter. Optionally, the recombinase gene is operably linked to an albumin promoter.

In some such non-human animals, non-human animal cells, or non-human animal genomes, the adaptor protein is at the N-terminal end of the chimeric adaptor protein, and the one or more transcriptional activation domains are at the C-terminal end of the chimeric adaptor protein. In some such non-human animals, non-human animal cells, or non-human animal genomes, the adaptor protein comprises an MS2 coat protein or a functional fragment or variant thereof. In some such non-human animals, non-human animal cells, or non-human animal genomes, the one or more transcriptional activation domains in the chimeric adaptor protein are selected from: VP16, VP64, p65, MyoD1, HSF1, RTA, SET7/9, and a combination thereof. Optionally, the one or more transcriptional activation domains in the chimeric adaptor protein comprise p65 and HSF1. Optionally, the chimeric adaptor protein comprises from N-terminus to C-terminus: an MS2 coat protein; a nuclear localization signal; the p65 transcriptional activation domain; and the HSF1 transcriptional activation domain. Optionally, the chimeric adaptor protein comprises a sequence at least 90%, at least 95%, at least 96%, at least 97%, at least 98%, at least 99%, or 100% identical to the sequence set forth in SEQ ID NO: 102. Optionally, the segment of the first expression cassette encoding the chimeric adaptor protein comprises a sequence at least 90%, at least 95%, at least 96%, at least 97%, at least 98%, at least 99%, or 100% identical to the sequence set forth in SEQ ID NO: 114.

In some such non-human animals, non-human animal cells, or non-human animal genomes, the first expression cassette is multicistronic. Optionally, the segment of the first expression cassette encoding the chimeric Cas protein is separated from the segment of the first expression cassette encoding the chimeric adaptor protein by an internal ribosome entry site (IRES). Optionally, the segment of the first expression cassette encoding the chimeric Cas protein is separated from the segment of the first expression cassette encoding the chimeric adaptor protein by a nucleic acid encoding a 2A peptide. Optionally, the 2A peptide is a T2A peptide.

In some such non-human animals, non-human animal cells, or non-human animal genomes, the first expression cassette is integrated into a safe harbor locus. In some such non-human animals, non-human animal cells, or non-human animal genomes, the first expression cassette and/or the second expression cassette is integrated into a safe harbor locus. Optionally, the non-human animal, non-human animal cell, or non-human animal genome is heterozygous for the first expression cassette and is heterozygous for the second expression cassette, and the first expression cassette is genomically integrated within a first allele of the safe harbor locus, and the second expression cassette is genomically integrated within a second allele of the safe harbor locus. Optionally, the safe harbor locus is a Rosa26 locus. Optionally, the first expression cassette is operably linked to an endogenous promoter in the safe harbor locus.

In some such non-human animals, non-human animal cells, or non-human animal genomes, serum levels of a TTR protein encoded by the humanized endogenous TTR locus are at least about 10 μg/mL, at least about 20 μg/mL, at least about 30 μg/mL, at least about 40 μg/mL, at least about 50 μg/mL, at least about 60 μg/mL, at least about 70 μg/mL, at least about 80 μg/mL, at least about 90 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/mL, at least about 500 μg/mL, at least about 600 μg/mL, at least about 700 μg/mL, at least about 800 μg/mL, at least about 900 μg/mL, or at least about 1000 μg/mL.

In another aspect, provided are humanized non-human animal TTR genes. In some such genes, a region of the non-human animal TTR gene comprising both a TTR exonic sequence and a TTR intronic sequence has been deleted and replaced with a corresponding human TTR sequence comprising both a TTR exonic sequence and a TTR intronic sequence, wherein the humanized non-human animal TTR gene comprises a V30M mutation.

In another aspect, provided are targeting vectors for generating a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous TTR locus comprises a V30M mutation, and wherein the targeting vector comprises an insert nucleic acid comprising the V30M mutation and the corresponding human TTR sequence flanked by a 5′ homology arm targeting a 5′ target sequence at the endogenous TTR locus and a 3′ homology arm targeting a 3′ target sequence at the endogenous TTR locus.

In another aspect, provided are methods of assessing the activity of a human-TTR-targeting reagent in vivo. Some such methods comprise: (a) administering the human-TTR-targeting reagent to any of the above non-human animals comprising a humanized TTR locus comprising a V30M mutation; and (b) assessing the activity of the human-TTR-targeting reagent in the non-human animal.

In some such methods, the activity of the human-TTR-targeting reagent is assessed compared to the non-human animal, non-human animal cell, or non-human animal genome prior to administering the human-TTR-targeting reagent. In some such methods, the activity of the human-TTR-targeting reagent is assessed compared to a control non-human animal, non-human animal cell, or non-human animal genome that has not been administered the human-TTR-targeting reagent.

Some such methods further comprise administering one or more guide RNAs or one or more DNAs encoding the one or more guide RNAs to the non-human animal, non-human animal cell, or non-human animal genome prior to step (a), wherein each of the one or more guide RNAs comprises one or more adaptor-binding elements to which the chimeric adaptor protein can specifically bind, and wherein each of the one or more guide RNAs forms a complex with the chimeric Cas protein and the chimeric adaptor protein and guides them to a target sequence within the humanized endogenous TTR locus, thereby increasing expression of the humanized endogenous TTR locus. Optionally, the human-TTR-targeting reagent is administered at least about 1 day, at least about 2 days, at least about 3 days, at least about 4 days, at least about 5 days, at least about 6 days, at least about 7 days, at least about 8 days, at least about 9 days, at least about 10 days, at least about 15 days, at least about 20 days, at least about 25 days, or at least about 30 days after administering the one or more guide RNAs or the one or more DNAs encoding the one or more guide RNAs.

Some such methods further comprise measuring expression of a Ttr messenger RNA encoded by the humanized endogenous TTR locus or measuring expression of a TTR protein encoded by the humanized endogenous TTR locus after administering the one or more guide RNAs or the one or more DNAs encoding the one or more guide RNAs and before administering the human-TTR-targeting reagent. Optionally, the human-TTR-targeting reagent is not administered until serum levels of the TTR protein encoded by the humanized endogenous TTR locus are at least about 10 μg/mL, at least about 20 μg/mL, at least about 30 μg/mL, at least about 40 μg/mL, at least about 50 μg/mL, at least about 60 μg/mL, at least about 70 μg/mL, at least about 80 μg/mL, at least about 90 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/mL, at least about 500 μg/mL, at least about 600 μg/mL, at least about 700 μg/mL, at least about 800 μg/mL, at least about 900 μg/mL, or at least about 1000 μg/mL. In some such methods, the human-TTR-targeting reagent is not administered until TTR amyloid deposition occurs.

In some such methods, the administering the one or more guide RNAs or the one or more DNAs encoding the one or more guide RNAs comprises adeno-associated virus (AAV)-mediated delivery, lipid nanoparticle (LNP)-mediated delivery, or hydrodynamic delivery (HDD). In some such methods, the administering the one or more guide RNAs or the one or more DNAs encoding the one or more guide RNAs comprises LNP-mediated delivery. Optionally, the LNP dose is between about 0.1 mg/kg and about 2 mg/kg. In some such methods, the administering the one or more guide RNAs or the one or more DNAs encoding the one or more guide RNAs comprises AAV8-mediated delivery.

In some such methods, the method comprises administering the one or more guide RNAs in the form of RNA. In some such methods, the method comprises administering the one or more DNAs encoding the one or more guide RNAs. Optionally, each of the one or more guide RNAs is operably linked to a different U6 promoter.

In some such methods, the target sequence comprises a regulatory sequence within the humanized endogenous TTR locus. Optionally, the regulatory sequence comprises a promoter or an enhancer. In some such methods, the target sequence is within 200 base pairs of the transcription start site of the humanized endogenous TTR locus. Optionally, the target sequence is within a region 200 base pairs upstream of the transcription start site and 1 base pair downstream of the transcription start site.

In some such methods, each of the one or guide RNAs comprises two adaptor-binding elements to which the chimeric adaptor protein can specifically bind. Optionally, a first adaptor-binding element is within a first loop of each of the one or more guide RNAs, and a second adaptor-binding element is within a second loop of each of the one or more guide RNAs. Optionally, each of one or more guide RNAs is a single guide RNA comprising a CRISPR RNA (crRNA) portion fused to a transactivating CRISPR RNA (tracrRNA) portion, and the first loop is the tetraloop corresponding to residues 13-16 of SEQ ID NO: 146, 148, 150, or 151, and the second loop is the stem loop 2 corresponding to residues 53-56 of SEQ ID NO: 146, 148, 150, or 151.

In some such methods, the adaptor-binding element comprises the sequence set forth in SEQ ID NO: 106. Optionally, each of the one or more guide RNAs comprises the sequence set forth in SEQ ID NO: 127, 132, 140, or 141.

In some such methods, one or more of the guide RNAs targets a sequence comprising the sequence set forth in any one of SEQ ID NOS: 121-123 or optionally wherein one or more of the guide RNAs comprises the sequence set forth in any one of SEQ ID NOS: 124-126. In some such methods, the one or more guide RNAs comprise multiple guide RNAs that target the humanized endogenous TTR locus. Optionally, the one or more guide RNAs comprise at least three guide RNAs that target the humanized endogenous TTR locus. Optionally, a first guide RNA targets a sequence comprising SEQ ID NO: 121 or comprises the sequence set forth in SEQ ID NO: 124, a second guide RNA targets a sequence comprising SEQ ID NO: 122 or comprises the sequence set forth in SEQ ID NO: 125, and a third guide RNA targets a sequence comprising SEQ ID NO: 123 or comprises the sequence set forth in SEQ ID NO: 126.

In some such methods, the administering of the human-TTR-targeting reagent comprises adeno-associated virus (AAV)-mediated delivery, lipid nanoparticle (LNP)-mediated delivery, hydrodynamic delivery (HDD), or injection. Optionally, the administering comprises LNP-mediated delivery. Optionally, the administering comprises AAV8-mediated delivery.

In some such methods, step (b) comprises assessing the activity of the human-TTR-targeting reagent in the liver of the non-human animal. In some such methods, step (b) comprises measuring expression of a TTR messenger RNA encoded by the humanized endogenous TTR locus. In some such methods, step (b) comprises measuring expression of a transthyretin protein encoded by the humanized endogenous TTR locus. Optionally, measuring expression of the transthyretin protein comprises measuring serum levels of the transthyretin protein in the non-human animal. Optionally, measuring expression of the transthyretin protein comprises measuring expression of the transthyretin protein in the liver of the non-human animal.

In some such methods, the human-TTR-targeting reagent is a genome-editing agent, and step (b) comprises assessing modification of the humanized endogenous TTR locus. Optionally, step (b) comprises measuring the frequency of insertions or deletions within the humanized endogenous TTR locus.

In some such methods, the human-TTR-targeting reagent comprises a nuclease agent designed to target a region of a human TTR gene. Optionally, the nuclease agent comprises a Cas protein and a guide RNA designed to target a guide RNA target sequence in the human TTR gene. Optionally, the Cas protein is a Cas9 protein.

In some such methods, the human-TTR-targeting reagent comprises an exogenous donor nucleic acid, wherein the exogenous donor nucleic acid is designed to target the human TTR gene, and optionally wherein the exogenous donor nucleic acid is delivered via AAV. In some such methods, the human-TTR-targeting reagent is an RNAi agent or an antisense oligonucleotide. In some such methods, the human-TTR-targeting reagent is an antigen-binding protein. In some such methods, the human-TTR-targeting reagent is small molecule.

In some such methods, assessing the activity of the human-TTR-targeting reagent in the non-human animal comprises assessing transthyretin activity. In some such methods, the assessing is in comparison to an untreated control non-human animal.

In some such methods, the method comprises administering exogenous, pre-formed transthyretin aggregates or fibrils to the non-human animal in step (a) or prior to step (a). Optionally, the exogenous, pre-formed transthyretin aggregates or fibrils comprise a V30M mutation. Optionally, the exogenous, pre-formed transthyretin aggregates or fibrils are human. In some such methods, the exogenous, pre-formed transthyretin aggregates or fibrils are administered to the non-human animal via intravenous injection. In some such methods, the exogenous, pre-formed transthyretin aggregates or fibrils are administered via hydrodynamic delivery. In some such methods, the exogenous, pre-formed transthyretin aggregates or fibrils are administered together with heparin.

In another aspect, provided are methods of optimizing the activity of a human-TTR-targeting reagent in vivo. Some such methods comprise: (I) performing any of the above methods of assessing the activity of a human-TTR-targeting reagent in vivo a first time in a first non-human animal; (II) changing a variable and performing the method of step (I) a second time with the changed variable in a second non-human animal; and (III) comparing the activity of the human-TTR-targeting reagent in step (I) with the activity of the human-TTR-targeting reagent in step (II), and selecting the method resulting in the higher activity.

In some such methods, the changed variable in step (II) is the delivery vehicle of introducing the human-TTR-targeting reagent into the non-human animal. In some such methods, the changed variable in step (II) is the route of administration of introducing the human-TTR-targeting reagent into the non-human animal. In some such methods, the changed variable in step (II) is the concentration or amount of the human-TTR-targeting reagent introduced into the non-human animal. In some such methods, the changed variable in step (II) is the form of the human-TTR-targeting reagent introduced into the non-human animal. In some such methods, the changed variable in step (II) is the human-TTR-targeting reagent introduced into the non-human animal.

In another aspect, provided are methods of making any of the above non-human animals comprising a humanized TTR locus comprising a V30M mutation.

Some such methods comprise: (a) introducing into a non-human animal host embryo a genetically modified non-human animal embryonic stem (ES) cell comprising in its genome a humanized endogenous TTR locus in which a segment of the endogenous TTR locus has been deleted and replaced with a corresponding human TTR sequence, wherein the humanized endogenous TTR locus comprises a V30M mutation; and (b) gestating the non-human animal host embryo in a surrogate mother, wherein the surrogate mother produces an F0 progeny genetically modified non-human animal comprising the humanized endogenous TTR locus comprising the V30M mutation. Some such methods further comprise modifying the genome of a non-human animal ES cell to comprise the humanized endogenous TTR locus comprising the V30M mutation prior to step (a). Some such methods comprise: (a) modifying the genome of a non-human animal one-cell stage embryo to comprise in its genome a humanized endogenous TTR locus comprising a V30M mutation and in which a segment of the endogenous TTR locus has been deleted and replaced with a corresponding human TTR sequence to produce a genetically modified non-human animal embryo; and (b) gestating the genetically modified non-human animal embryo in a surrogate mother, wherein the surrogate mother produces an F0 progeny genetically modified non-human animal comprising the humanized endogenous TTR locus comprising the V30M mutation. Some such methods further comprise crossing the F0 progeny genetically modified non-human animal comprising the humanized endogenous TTR locus comprising the V30M mutation with a non-human animal comprising a genomically integrated expression cassette comprising a nucleic acid encoding a chimeric Clustered Regularly Interspaced Short Palindromic Repeats (CRISPR) associated (Cas) protein comprising a nuclease-inactive Cas protein fused to one or more transcriptional activation domains and further comprising a nucleic acid encoding a chimeric adaptor protein comprising an adaptor protein fused to one or more transcriptional activation domains.

Some such methods comprise: (a) introducing into a non-human animal embryonic stem (ES) cell a targeting vector comprising a nucleic acid insert comprising the V30M mutation and the human TTR sequence flanked by a 5′ homology arm corresponding to a 5′ target sequence in the endogenous TTR locus and a 3′ homology arm corresponding to a 3′ target sequence in the endogenous TTR locus, wherein the targeting vector recombines with the endogenous TTR locus to produce a genetically modified non-human ES cell comprising in its genome the humanized endogenous TTR locus comprising the human TTR sequence and the V30M mutation; (b) introducing the genetically modified non-human ES cell into a non-human animal host embryo; and (c) gestating the non-human animal host embryo in a surrogate mother, wherein the surrogate mother produces an F0 progeny genetically modified non-human animal comprising in its genome the humanized endogenous TTR locus comprising the human TTR sequence. Optionally, the targeting vector is a large targeting vector at least 10 kb in length or in which the sum total of the 5′ and 3′ homology arms is at least 10 kb in length.

Some such methods comprise: (a) introducing into a non-human animal one-cell stage embryo a targeting vector comprising a nucleic acid insert comprising the V30M mutation and the human TTR sequence flanked by a 5′ homology arm corresponding to a 5′ target sequence in the endogenous TTR locus and a 3′ homology arm corresponding to a 3′ target sequence in the endogenous TTR locus, wherein the targeting vector recombines with the endogenous TTR locus to produce a genetically modified non-human one-cell stage embryo comprising in its genome the humanized endogenous TTR locus comprising the human TTR sequence and the V30M mutation; and (b) gestating the genetically modified non-human animal one-cell stage embryo in a surrogate mother to produce a genetically modified F0 generation non-human animal comprising in its genome the humanized endogenous TTR locus comprising the human TTR sequence.

In some such methods, step (a) further comprises introducing a nuclease agent or a nucleic acid encoding the nuclease agent, wherein the nuclease agent targets a target sequence in the endogenous TTR locus. Optionally, the nuclease agent comprises a Cas protein and a guide RNA. Optionally, the Cas protein is a Cas9 protein. Optionally, step (a) further comprises introducing a second guide RNA or a DNA encoding the second guide RNA, wherein the second guide RNA targets a second target sequence within the endogenous TTR locus. Optionally, step (a) further comprises introducing a third guide RNA or a DNA encoding the third guide RNA, wherein the third guide RNA targets a third target sequence within the endogenous TTR locus, and a fourth guide RNA or a DNA encoding the fourth guide RNA, wherein the fourth guide RNA targets a fourth target sequence within the endogenous TTR locus.

In some such methods, the non-human animal is a mouse or a rat. Optionally, the non-human animal is a mouse.

In another aspect, provided are methods of making any of the above non-human animals comprising a humanized TTR locus comprising a V30M mutation and comprising in their genome a genomically integrated expression cassette, wherein the expression cassette comprises: (a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; and (b) a nucleic acid encoding a chimeric adaptor protein comprising an adaptor protein fused to one or more transcriptional activation domains. Some such methods comprise: (a) introducing into a non-human animal host embryo a genetically modified non-human animal embryonic stem (ES) cell comprising in its genome: (i) a humanized endogenous TTR locus in which a segment of the endogenous TTR locus has been deleted and replaced with a corresponding human TTR sequence, wherein the humanized endogenous TTR locus comprises a V30M mutation; and (ii) a genomically integrated expression cassette comprising a nucleic acid encoding a Cas protein comprising a nuclease-inactive Cas protein fused to one or more transcriptional activation domains and a nucleic acid encoding a chimeric adaptor protein comprising an adaptor protein fused to one or more transcriptional activation domains; and (b) gestating the non-human animal host embryo in a surrogate mother, wherein the surrogate mother produces an F0 progeny genetically modified non-human animal comprising the humanized endogenous TTR locus and the genomically integrated expression cassette. Some such methods further comprise modifying the genome of a non-human animal ES cell to comprise the humanized endogenous TTR locus comprising the V30M mutation and the genomically integrated expression cassette prior to step (a). In some such methods, the non-human animal is a mouse or a rat. Optionally, the non-human animal is a mouse.

In another aspect, provided are methods of accelerating transthyretin amyloid deposition in a non-human animal, comprising administering exogenous, pre-formed transthyretin aggregates or fibrils to any of the above non-human animals or non-human animal cells comprising a humanized TTR locus comprising a V30M mutation. Optionally, the exogenous, pre-formed transthyretin aggregates or fibrils comprise a V30M mutation. Optionally, the exogenous, pre-formed transthyretin aggregates or fibrils are human. In some such methods, the exogenous, pre-formed transthyretin aggregates or fibrils are administered to the non-human animal via intravenous injection. In some such methods, the exogenous, pre-formed transthyretin aggregates or fibrils are administered via hydrodynamic delivery. In some such methods, the exogenous, pre-formed transthyretin aggregates or fibrils are administered together with heparin.

### BRIEF DESCRIPTION OF THE FIGURES

FIG. 1 shows an alignment of human and mouse wild type transthyretin (TTR) precursor proteins (SEQ ID NOS: 1 and 13, respectively). The signal peptide, T4 binding domain, phase 0 exon/intron boundaries, and phase 1/2 exon/intron boundaries are denoted, along with the position of the V30M mutation.

FIG. 2 shows schematics (not drawn to scale) of the wild-type murine Ttr locus and a mutant humanized mouse TTR locus (human V30M TTR). Exons, introns, 5′ untranslated regions (UTRs), 3′ UTRs, start codons (ATG), stop codons (TGA), and loxP scars from selection cassettes are denoted. White boxes indicate murine sequence; black boxes indicate human sequence.

FIG. 3 shows a schematic (not drawn to scale) of the targeting to create the mutant (V30M) humanized mouse TTR locus. The wild type mouse Ttr locus, the F0 allele of the mutant humanized mouse TTR locus with the self-deleting neomycin (SDC-Puro) selection cassette (MAID 8526), and the F1 allele of the mutant humanized mouse TTR locus with the loxP scar from removal of the SDC-Puro selection cassette (MAID 8527) are shown. White boxes indicate murine sequence; black boxes indicate human sequence.

FIG. 4 shows a schematic (not drawn to scale) of the strategy for screening of the humanized mouse TTR locus, including loss-of-allele assays (7576mTU, 4552mTU, 9212mTU, 7655mTU, 9090mTM, 7576mTD, 9212mTGD, and 7655mTD), gain of allele assays (7576hTU, 7655hTU, 7576hTD, Puro), retention assays (9204mretU, 9090retU, 9090retU2, 9090retU3, 9090retD, 9090retD2, 9090retD3, 9204mretD), and CRISPR assays designed to cover the region that is disrupted by the CRISPR guides (9090mTGU and 9090mTGD). White boxes indicate murine sequence; black boxes indicate human sequence.

FIG. 5 shows results of an ELISA assaying human TTR levels in blood plasma samples of wild type humanized TTR and V30M humanized TTR mice.

FIG. 6A (not to scale) shows a lox-stop-lox (LSL) dCas9 synergistic activation mediator (SAM) allele (LSL-SAM allele), comprising from 5′ to 3′: a 3′ splicing sequence; a first loxP site; a neomycin resistance gene; a polyadenylation signal; a second loxP site; a dCas9-NLS-VP64 coding sequence (NLS-dCas9-NLS-VP64); a T2A peptide coding sequence; an MCP-NLS-p65-HSF1 coding sequence; and a Woodchuck hepatitis virus posttranscriptional regulatory element (WPRE).

FIG. 6B (not to scale) shows the allele from FIG. 6A with the floxed neomycin resistance gene and polyadenylation signal removed (SAM allele).

FIG. 7 (not to scale) shows a general schematic for targeting the LSL-SAM allele from FIG. 6A into the first intron of the Rosa26 (R26) locus.

FIG. 8 (not to scale) shows a schematic for introducing a guide RNA array allele into R26^{SAM/+} mouse embryonic stem cells. The guide RNA array allele comprises from 5′ to 3′: a 3′ splicing sequence; a first rox site; a puromycin resistance gene; a polyadenylation signal; a second rox site; a first U6 promoter; a first guide RNA coding sequence; a second U6 promoter; a second guide RNA coding sequence; a third U6 promoter; and a third guide RNA coding sequence.

FIG. 9 (not to scale) shows a schematic for designing three guide RNAs that target upstream of the transcription start site of Ttr.

FIG. 10 shows a schematic of a generic single guide RNA (SEQ ID NO: 132) in which the tetraloop and stem loop 2 have been replaced with MS2-binding aptamers to facilitate recruitment of chimeric MS2 coat protein (MCP) fused to transcriptional activation domains.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” (amino-terminus) and a “C-terminus” (carboxy-terminus or carboxyl-terminus). The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), lipid droplets, proteins, and nucleic acids includes cells, tissues (e.g., liver samples), lipid droplets, proteins, and nucleic acids that are relatively purified with respect to other bacterial, viral, cellular, or other components that may normally be present in situ, up to and including a substantially pure preparation of the cells, tissues (e.g., liver samples), lipid droplets, proteins, and nucleic acids. The term “isolated” also includes cells, tissues (e.g., liver samples), lipid droplets, proteins, and nucleic acids that have no naturally occurring counterpart, have been chemically synthesized and are thus substantially uncontaminated by other cells, tissues (e.g., liver samples), lipid droplets, proteins, and nucleic acids, or has been separated or purified from most other components (e.g., cellular components or organism components) with which they are naturally accompanied (e.g., other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a rat cell or rat. For example, an endogenous Ttr sequence of a mouse refers to a native Ttr sequence that naturally occurs at the Ttr locus in the mouse.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a TTR protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “TTR locus” may refer to the specific location of a TTR gene, TTR DNA sequence, TTR-encoding sequence, or TTR position on a chromosome of the genome of an organism that has been identified as to where such a sequence resides. A “TTR locus” may comprise a regulatory element of a TTR gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

The “coding region” or “coding sequence” of a gene consists of the portion of a gene's DNA or RNA, composed of exons, that codes for a protein. The region begins at the start codon on the 5′ end and ends at the stop codon on the 3′ end.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid, or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., an organism or body or a cell or tissue within an organism or body) and to processes or reactions that occur within a natural environment. The term “ex vivo” includes cells that have been removed from the body of an individual and processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Repair in response to double-strand breaks (DSBs) occurs principally through two conserved DNA repair pathways: homologous recombination (HR) and non-homologous end joining (NHEJ). See Kasparek & Humphrey (2011) Semin. Cell Dev. Biol. 22(8):886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLoS One 7:e45768:1-9; and Wang et al. (2013) Nat. Biotechnol. 31:530-532, each of which is herein incorporated by reference in its entirety for all purposes.

Non-homologous end joining (NHEJ) includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range.

Unless otherwise apparent from the context, the term “about” encompasses values±5 of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

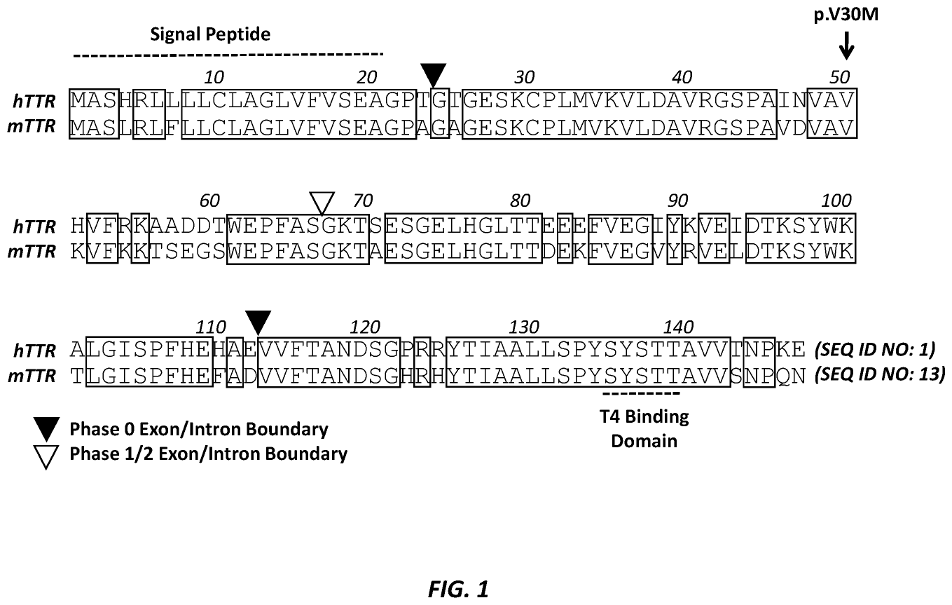

Disclosed herein are non-human animal genomes, non-human animal cells, and non-human animals comprising a humanized TTR locus comprising a V30M mutation and methods of making and using such non-human animal cells and non-human animals. Some such non-human animal genomes, non-human animal cells, and non-human animals further comprise CRISPR/Cas synergistic activation mediator system components. For example, disclosed herein are non-human animal genomes, non-human animal cells, and non-human animals comprising in their genome a Clustered Regularly Interspersed Short Palindromic Repeats (CRISPR)/CRISPR-associated (Cas)-based synergistic activation mediator (SAM) expression cassette and a humanized TTR locus comprising a V30M mutation and methods of using such non-human animal cells and non-human animals. A TTR locus comprising a V30M mutation refers to a TTR locus that encodes a TTR protein comprising a V30M mutation or comprising a mutation corresponding to the V30M mutation in human TTR when the encoded TTR protein is optimally aligned (greatest number of perfectly matched residues) with human TTR. TTR V30M is the most common mutation associated with familial amyloid polyneuropathy (FAP). The clinical presentation of TTR amyloidosis can differ according to the underlying TTR mutation, and the predominant pathogenic phenotype associated with TTR V30M is neuropathy. The nomenclature of the amino acid position for the V30M mutation refers to the position of the mutation in the mature human TTR protein after cleavage of the 20 amino acid signal peptide. See FIG. 1. This nomenclature is consistent with nomenclature used in publications describing this mutation.

Also disclosed herein are humanized non-human animal TTR genes comprising a V30M mutation and a targeted genetic modification that humanizes the non-human animal TTR genes and nuclease agents and targeting vectors for use in humanizing a non-human animal TTR gene and/or introducing a V30M mutation. Also disclosed herein are isolated liver samples (e.g., fractioned liver samples) prepared from the non-human animals comprising a humanized TTR locus comprising a V30M mutation.

Non-human animal cells or non-human animals comprising a humanized TTR locus comprising a V30M mutation express a human transthyretin protein (e.g., human transthyretin precursor protein) or a chimeric transthyretin protein (e.g., chimeric transthyretin precursor protein) comprising one or more fragments of a human transthyretin protein (e.g., human transthyretin precursor protein). Such non-human animal cells and non-human animals can be used to assess delivery or efficacy of human-TTR-targeting agents (e.g., CRISPR/Cas9 genome editing agents) in vitro or ex vivo or in vivo and can be used in methods of optimizing the delivery of efficacy of such agents in vitro or ex vivo or in vivo. When the SAM expression cassettes are present, they can be used to upregulate transcription of target genes such as the humanized TTR genes comprising a V30M mutation as disclosed herein in vitro, ex vivo or in vivo in order to achieve, for example, higher TTR expression levels that reach human physiological levels. SAM activation can be tuned to a more representative level of normal human expression or exacerbated above the disease state to facilitate a thorough characterization of ATTR models.

In some of the non-human animal cells and non-human animals disclosed herein, some or most or all of the human TTR genomic DNA is inserted into the corresponding orthologous non-human animal TTR locus. In some of the non-human animal cells and non-human animals disclosed herein, some or most or all of the non-human animal genomic DNA is replaced one-for-one with corresponding orthologous human genomic DNA. Compared to non-human animals with cDNA insertions, expression levels should be higher when the intron-exon structure and splicing machinery are maintained because conserved regulator elements are more likely to be left intact, and spliced transcripts that undergo RNA processing are more stable than cDNAs. In contrast, insertion of human TTR cDNA (e.g., along with insertion of an artificial beta-globin intron in the 5′ UTR) into a non-human animal TTR locus would abolish conserved regulatory elements such as those contained within the first exon and intron of the non-human animal TTR. Replacing the non-human animal genomic sequence with the corresponding orthologous human genomic sequence is more likely to result in faithful expression of the transgene from the endogenous TTR locus. Similarly, transgenic non-human animals with transgenic insertion of human-TTR-coding sequences at a random genomic locus rather than the endogenous non-human-animal TTR locus will not as accurately reflect the endogenous regulation of TTR expression. A humanized TTR allele resulting from replacing most or all of the non-human animal genomic DNA one-for-one with corresponding orthologous human genomic DNA or inserting human TTR genomic sequence in the corresponding orthologous non-human TTR locus will provide the true human target or a close approximation of the true human target of human-TTR-targeting reagents (e.g., CRISPR/Cas9 reagents designed to target human TTR), thereby enabling testing of the efficacy and mode of action of such agents in live animals as well as pharmacokinetic and pharmacodynamics studies in a setting where the humanized protein and humanized gene are the only version of TTR present.

The methods and compositions disclosed herein can optionally employ non-human animal genomes, non-human animal cells, and non-human animals comprising chimeric Cas protein expression cassettes, chimeric adaptor protein expression cassettes, or synergistic activation mediator (SAM) expression cassettes (e.g., a chimeric Cas protein coding sequence and a chimeric adaptor protein sequence) so that the components can be constitutively available or, for example, available in a tissue-specific or temporal-specific manner. The cassettes can be genomically integrated. Such genomes, cells, and non-human animals can also comprise guide RNA expression cassettes (e.g., Ttr guide RNA expression cassettes or Ttr guide RNA array expression cassettes) and/or recombinase expression cassettes as disclosed elsewhere herein. Alternatively, one or more components (e.g., guide RNAs and/or recombinases) can be introduced into the cells and non-human animals by other means to induce transcriptional activation of a target gene (e.g., the humanized Ttr gene).

Non-human animals comprising the SAM expression cassettes simplify the process for upregulating expression of a target gene (e.g., the humanized Ttr gene) in vivo because only the guide RNAs need to be introduced into the non-human animal to activate transcription of a target gene. If the non-human animal also comprises a guide RNA expression cassette, the effects of target gene activation or upregulation can be studied without introducing any further components. In addition, the SAM expression cassettes or guide RNA expression cassettes can optionally be conditional expression cassettes that can be selectively expressed in particular tissues or developmental stages, which can, for example, reduce the risk of Cas-mediated toxicity in vivo. Alternatively, such expression cassettes can be constitutively expressed to enable testing of activity in any and all types of cells, tissues, and organs.

Non-human animal genomes, non-human animal cells, and non-human animals comprising a humanized TTR locus comprising a V30M mutation as described elsewhere herein and one or more nucleic acids encoding a chimeric Cas protein, a chimeric adaptor protein, a guide RNA, a recombinase, or any combination thereof (any combination of such SAM system nucleic acids) are provided. The genomes, cells, or non-human animals can be male or female.

The genomes, cells, or non-human animals can be heterozygous or homozygous for the humanized TTR locus comprising the V30M mutation. A diploid organism has two alleles at each genetic locus. Each pair of alleles represents the genotype of a specific genetic locus. Genotypes are described as homozygous if there are two identical alleles at a particular locus and as heterozygous if the two alleles differ. A non-human animal comprising a humanized TTR locus comprising a V30M mutation described herein can comprise the humanized TTR locus in its germline.

The SAM nucleic acids or expression cassettes can be stably integrated into the genome (i.e., into a chromosome) of the cell or non-human animal or can be located outside of a chromosome (e.g., extrachromosomally replicating DNA). The SAM nucleic acids or expression cassettes can be randomly integrated into the genome of the non-human animal (i.e., transgenic, or can be integrated into a predetermined region (e.g., a safe harbor locus) of the genome of the non-human animal (i.e., knock in). The target genomic locus at which a SAM nucleic acid or expression cassette is stably integrated can be heterozygous for the nucleic acid or expression cassette or homozygous for the nucleic acid or expression cassette. A non-human animal comprising a stably integrated SAM nucleic acid or expression cassette described herein can comprise the nucleic acid or expression cassette in its germline.

For example, a non-human animal genome, non-human animal cell, or non-human animal can comprise a chimeric Cas protein expression cassette, a chimeric adaptor protein expression cassette, or a synergistic activation mediator (SAM) expression cassette (comprising both a chimeric Cas protein coding sequence and a chimeric adaptor protein sequence) as disclosed herein. In one example, the genome, cell, or non-human animal comprises a SAM expression cassette comprising both a chimeric Cas protein coding sequence and a chimeric adaptor protein coding sequence. In one example, the SAM expression cassette (or chimeric Cas protein expression cassette or chimeric adaptor protein expression cassette) is stably integrated into the genome. The stably integrated SAM expression cassette (or chimeric Cas protein expression cassette or chimeric adaptor protein expression cassette) can be randomly integrated into the genome of the non-human animal (i.e., transgenic), or it can be integrated into a predetermined region of the genome of the non-human animal (i.e., knock in). In one example, the SAM expression cassette (or chimeric Cas protein expression cassette or chimeric adaptor protein expression cassette) is stably integrated into a predetermined region of the genome, such as a safe harbor locus (e.g., Rosa26). The target genomic locus at which the SAM expression cassette (or chimeric Cas protein expression cassette or chimeric adaptor protein expression cassette) is stably integrated can be heterozygous or homozygous for the SAM expression cassette (or chimeric Cas protein expression cassette or chimeric adaptor protein expression cassette).

Optionally, the genome, cell, or non-human animal described above can further comprise a guide RNA expression cassette (e.g., guide RNA array expression cassette). The guide RNA expression cassette can be stably integrated into the genome (i.e., into a chromosome) of the cell or non-human animal or it can be located outside of a chromosome (e.g., extrachromosomally replicating DNA or introduced into the cell or non-human animal via AAV, LNP, or any other means disclosed herein). The guide RNA expression cassette can be randomly integrated into the genome of the non-human animal (i.e., transgenic), or it can be integrated into a predetermined region (e.g., a safe harbor locus) of the genome of the non-human animal (i.e., knock in). The target genomic locus at which the guide RNA expression cassette is stably integrated can be heterozygous or homozygous for the guide RNA expression cassette. In one example, a genome, cell, or non-human animal comprises both a SAM expression cassette (or chimeric Cas protein expression cassette or chimeric adaptor protein expression cassette) and a guide RNA expression cassette. In one example, both cassettes are genomically integrated. The guide RNA expression cassette can be integrated at a different target genomic locus from the SAM expression cassette (or chimeric Cas protein expression cassette or chimeric adaptor protein expression cassette), or it can be genomically integrated at the same target locus (e.g., a Rosa26 locus, such as integrated in the first intron of the Rosa26 locus). For example, the genome, cell, or non-human animal can be heterozygous for each of a SAM expression cassette (or chimeric Cas protein expression cassette or chimeric adaptor protein expression cassette) and the guide RNA expression cassette, with one allele of the target genomic locus (e.g., Rosa26) comprising the SAM expression cassette (or chimeric Cas protein expression cassette or chimeric adaptor protein expression cassette), and a second allele of the target genomic locus (e.g., Rosa26) comprising the guide RNA expression cassette expression cassette.

Optionally, any of the genomes, cells, or non-human animals described above can further comprise a recombinase expression cassette. The recombinase expression cassette can be stably integrated into the genome (i.e., into a chromosome) of the cell or non-human animal or it can be located outside of a chromosome (e.g., extrachromosomally replicating DNA or introduced into the cell or non-human animal via AAV, LNP, HDD, or any other means disclosed herein). The recombinase expression cassette can be randomly integrated into the genome of the non-human animal (i.e., transgenic), or it can be integrated into a predetermined region (e.g., a safe harbor locus) of the genome of the non-human animal (i.e., knock in). The target genomic locus at which the recombinase expression cassette is stably integrated can be heterozygous or homozygous for the recombinase expression cassette. The recombinase expression cassette can be integrated at a different target genomic locus from any of the other expression cassettes disclosed herein, or it can be genomically integrated at the same target locus (e.g., a Rosa26 locus, such as integrated in the first intron of the Rosa26 locus).

### II. Non-Human Animals Comprising a Humanized TTR Locus Comprising a V30M Mutation

The non-human animal genomes, non-human animal cells, and non-human animals disclosed herein comprise a humanized TTR locus comprising a V30M mutation. Cells or non-human animals comprising a humanized TTR locus comprising a V30M mutation express a human transthyretin protein (e.g., human transthyretin precursor protein) comprising a V30M mutation or a partially humanized, chimeric transthyretin protein (e.g., chimeric transthyretin precursor protein) in which one or more fragments of the native transthyretin protein (e.g., native transthyretin precursor protein) have been replaced with corresponding fragments from human transthyretin (e.g., human transthyretin precursor protein), wherein the partially humanized, chimeric transthyretin protein (e.g., chimeric transthyretin precursor protein) comprises a V30M mutation. A TTR locus comprising a V30M mutation refers to a TTR locus that encodes a TTR protein comprising a V30M mutation or comprising a mutation corresponding to the V30M mutation in human TTR when the encoded TTR protein is optimally aligned (greatest number of perfectly matched residues) with human TTR. TTR V30M is the most common mutation associated with familial amyloid polyneuropathy (FAP). The nomenclature of the amino acid position for the V30M mutation refers to the position of the mutation in the mature human TTR protein after cleavage of the 20 amino acid signal peptide. This nomenclature is consistent with nomenclature used in publications describing this mutation. See FIG. 1.

A. Transthyretin (TTR)

The cells and non-human animals described herein comprise a humanized transthyretin (TTR) locus comprising a V30M mutation. Transthyretin (TTR) is a 127-amino acid, 55 kDa serum and cerebrospinal fluid transport protein primarily synthesized by the liver but also produced by the choroid plexus. It has also been referred to as prealbumin, thyroxine binding prealbumin, ATTR, TBPA, CTS, CTS1, HEL111, HsT2651, and PALB. In its native state, TTR exists as a tetramer. In homozygotes, homo-tetramers comprise identical 127-amino-acid beta-sheet-rich subunits. In heterozygotes, TTR tetramers can be made up of variant and/or wild-type subunits, typically combined in a statistical fashion. TTR is responsible for carrying thyroxine (T4) and retinol-bound RBP (retinol-binding protein) in both the serum and the cerebrospinal fluid.

Unless otherwise apparent from context, reference to human transthyretin (TTR) or its fragments or domains includes the natural, wild type human amino acid sequences including isoforms and allelic variants thereof. Transthyretin precursor protein includes a signal sequence (typically 20 amino acids), whereas the mature transthyretin protein does not. Exemplary human TTR precursor protein sequences are designated by Accession Numbers NP_000362.1 (NCBI) and P02766.1 (UniProt) (identical, each set forth SEQ ID NO: 1). Residues may be numbered according to UniProt Accession No. P02766.1, with the first amino acid of the mature protein (i.e., not including the 20 amino acid signal sequence) designated residue 1. In any other human TTR protein, residues are numbered according to the corresponding residues in UniProt Accession No. P02766.1 on maximum alignment. An exemplary human TTR precursor protein sequence comprising a V30M mutation is set forth in SEQ ID NO: 2. Exemplary human mature TTR protein sequences are set forth in SEQ ID NO: 4 (wild type) and SEQ ID NO: 5 (V30M). The full-length human TTR precursor protein set forth in SEQ ID NO: 1 has 147 amino acids, including a signal peptide (amino acids 1-20) and a mature TTR protein (amino acids 21-147). Delineations between these domains are as designated in UniProt. Reference to human TTR includes the canonical (wild type) forms as well as all allelic forms and isoforms. Any other forms of human TTR have amino acids numbered for maximal alignment with the wild type form, aligned amino acids being designated the same number.

The human TTR gene is located on chromosome 18 and includes four exons and three introns. An exemplary wild type human TTR gene is from residues 5001-12258 in the sequence designated by GenBank Accession No. NG_009490.1 (SEQ ID NO: 12). The four exons in SEQ ID NO: 12 include residues 1-205, 1130-1260, 3354-3489, and 6802-7258, respectively. The TTR coding sequence in SEQ ID NO: 12 includes residues 137-205, 1130-1260, 3354-3489, and 6802-6909. An exemplary wild type human TTR mRNA is designated by NCBI Accession No. NM_000371.3 (SEQ ID NO: 11). An exemplary wild type human TTR coding sequence is set forth in SEQ ID NO: 6. An exemplary human TTR coding sequence encoding a TTR protein comprising a V30M mutation is set forth in SEQ ID NO: 7.

The mouse Ttr gene is located and chromosome 18 and also includes four exons and three introns. An exemplary mouse Ttr gene is from residues 20665250 to 20674326 the sequence designated by GenBank Accession No. NC_000084.6 (SEQ ID NO: 20). The four exons in SEQ ID NO: 20 include residues 1-258, 1207-1337, 4730-4865, and 8382-9077, respectively. The Ttr coding sequence in SEQ ID NO: 20 includes residues 190-258, 1207-1337, 4730-4865, and 8382-8489. An exemplary mouse wild type TTR precursor protein is designated by UniProt Accession No. P07309.1 or NCBI Accession No. NP_038725.1 (identical, each set forth SEQ ID NO: 13). An exemplary mouse wild type mature TTR protein sequence is set forth in SEQ ID NO: 15. The full-length mouse TTR precursor protein set forth in SEQ ID NO: 13 has 147 amino acids, including a signal peptide (amino acids 1-20) and a mature TTR protein (amino acids 21-147). Delineations between these domains are as designated in UniProt. Reference to mouse TTR includes the canonical (wild type) forms as well as all allelic forms and isoforms. Any other forms of mouse TTR have amino acids numbered for maximal alignment with the wild type form, aligned amino acids being designated the same number. An exemplary mouse Ttr mRNA is designated by NCBI Accession No. NM_013697.5 (SEQ ID NO: 19). An exemplary mouse Ttr coding sequence is set forth in SEQ ID NO: 16.

An exemplary rat TTR protein is designated by UniProt Accession No. P02767 (NCBI GeneID 24856). An exemplary pig TTR protein is designated by UniProt Accession No. P50390 (NCBI GeneID 397419). An exemplary chicken TTR protein is designated by UniProt Accession No. P27731 (NCBI GeneID 396277). An exemplary cow TTR protein is designated by UniProt Accession No. O46375 (NCBI GeneID 280948). An exemplary sheep TTR protein is designated by UniProt Accession No. P12303 (NCBI GeneID 443389). An exemplary chimpanzee TTR protein designated by UniProt Accession No. Q5U7I5 (NCBI GeneID 493188). An exemplary orangutan TTR protein is designated by UniProt Accession No. Q5NVS2 (NCBI GeneID 100174094). An exemplary rabbit TTR protein is designated by UniProt Accession No. P07489. An exemplary cynomolgus monkey (macaque) TTR protein is designated by UniProt Accession No. Q8HW1 (NCBI GeneID 101864775).

Transthyretin (TTR) amyloidosis is a systemic disorder characterized by pathogenic, misfolded TTR and the extracellular deposition of amyloid fibrils composed of TTR. TTR amyloidosis is generally caused by destabilization of the native TTR tetramer form (due to environmental or genetic conditions), leading to dissociation, misfolding, and aggregation of TTR into amyloid fibrils that accumulate in various organs and tissues, causing progressive dysfunction. The dissociated monomers have a propensity to form misfolded protein aggregates and amyloid fibrils.

In humans, both wild-type TTR tetramers and mixed tetramers made up of mutant and wild-type subunits can dissociate, misfold, and aggregate, with the process of amyloidogenesis leading to the degeneration of post-mitotic tissue. Thus, TTR amyloidoses encompass diseases caused by pathogenic misfolded TTR resulting from mutations in TTR or resulting from non-mutated, misfolded TTR.

Senile systemic amyloidosis (SSA) and senile cardiac amyloidosis (SCA) are age-related types of amyloidosis that result from the deposition of wild-type TTR amyloid outside and within the cardiomyocytes of the heart. TTR amyloidosis is also the most common form of hereditary (familial) amyloidosis, which is caused by mutations that destabilize the TTR protein. TTR amyloidoses associated with point mutations in the TTR gene include familial amyloid polyneuropathy (FAP), familial amyloid cardiomyopathy (FAC), and central nervous system selective amyloidosis (CNSA). The most common FAP-associated mutation is TTR V30M.

B. Humanized TTR Loci Comprising a V30M Mutation

Disclosed herein are humanized endogenous TTR loci in which a segment of an endogenous Ttr locus has been deleted and replaced with a corresponding human TTR sequence (e.g., a corresponding human TTR genomic sequence), wherein a humanized TTR protein is expressed from the humanized endogenous TTR locus. The humanized TTR loci described herein comprise a V30M mutation. A TTR locus comprising a V30M mutation refers to a TTR locus that encodes a TTR protein comprising a V30M mutation or comprising a mutation corresponding to the V30M mutation in human TTR when the encoded TTR protein is optimally aligned (greatest number of perfectly matched residues) with human TTR. A residue (e.g., nucleotide or amino acid) in an endogenous TTR gene (or TTR protein) can be determined to correspond with a residue in the human TTR gene (or TTR protein) by optimally aligning the two sequences for maximum correspondence over a specified comparison window (e.g., the TTR coding sequence), wherein the portion of the polynucleotide (or amino acid) sequence in the comparison window may comprise additions or deletions (i.e., gaps) as compared to the reference sequence (which does not comprise additions or deletions) for optimal alignment of the two sequences. Two residues correspond if they are located at the same position when optimally aligned.

TTR V30M is the most common mutation associated with familial amyloid polyneuropathy (FAP). The nomenclature of the amino acid position for the V30M mutation refers to the position of the mutation in the mature human TTR protein after cleavage of the 20 amino acid signal peptide. See FIG. 1. This nomenclature is consistent with nomenclature used in publications describing this mutation. That is, the numbering of the residues here and below refers to numbering in the mature human transthyretin protein without the signal peptide (e.g., beginning at residue 21 of the transthyretin precursor protein, so this residue in the transthyretin precursor protein would be residue 50).

A humanized TTR locus can be a TTR locus in which the entire TTR gene is replaced with the corresponding orthologous human TTR sequence, it can be a TTR locus in which only a portion of the TTR gene is replaced with the corresponding orthologous human TTR sequence (i.e., humanized), it can be a TTR locus in which a portion of an orthologous human TTR locus is inserted (e.g., a humanized TTR locus can comprise human TTR sequence inserted into an endogenous TTR locus without replacing the corresponding orthologous endogenous sequence), or it can be a TTR locus in which a portion of the TTR gene is deleted and a portion of the orthologous human TTR locus is inserted. The portion of the orthologous human TTR locus that is inserted can, for example, comprise more of the human TTR locus than is deleted from the endogenous TTR locus. If only a portion of the TTR locus is humanized, the V30M mutation can be in the remaining endogenous TTR sequence or in the inserted orthologous human TTR sequence. A human TTR sequence corresponding to a particular segment of endogenous TTR sequence refers to the region of human TTR that aligns with the particular segment of endogenous TTR sequence when human TTR and the endogenous TTR are optimally aligned (greatest number of perfectly matched residues). The corresponding orthologous human sequence can comprise, for example, complementary DNA (cDNA) or genomic DNA. Optionally, a codon-optimized version of the corresponding orthologous human TTR sequence can be used and is modified to be codon-optimized based on codon usage in the non-human animal. Replaced or inserted (i.e., humanized) regions can include coding regions such as an exon, non-coding regions such as an intron, an untranslated region, or a regulatory region (e.g., a promoter, an enhancer, or a transcriptional repressor-binding element), or any combination thereof. As one example, exons corresponding to 1, 2, 3, or all 4 exons (or all or portions of 1, 2, 3, or all 4 exons) of the human TTR gene can be humanized. In a specific example, exons corresponding to exons 2 and 3 and the coding regions of exons 1 and 4 (i.e., not including the 5′ UTR and the 3′ UTR) can be deleted from the endogenous TTR locus, and a region of the human TTR gene including exons 2-4 and the coding region of exon 1 (i.e., not including the 5′ UTR) of the human TTR gene can be inserted. In a specific example, exons corresponding to exons 2 and 3 and the coding regions of exons 1 and 4 (i.e., not including the 5′ UTR and the 3′ UTR) can be deleted from the endogenous TTR locus, and a region of the human TTR gene including exons 2 and 3 and the coding regions of exons 1 and 4 as well as all or part of the 3′ UTR (i.e., not including the 5′ UTR) of the human TTR gene can be inserted. Alternatively, a region of TTR encoding an epitope recognized by an anti-human-TTR antigen-binding protein or a region targeted by human-TTR-targeting reagent (e.g., a small molecule) can be humanized. Likewise, introns corresponding to 1, 2, or all 3 introns of the human TTR gene can be humanized or can remain endogenous. In one example, introns corresponding to all 3 introns of the human TTR gene can be humanized (e.g., deleted from the endogenous locus and replaced with the corresponding human introns).

In a specific example, a humanized TTR locus can be one in which a region of the endogenous TTR locus has been deleted and replaced with an orthologous human TTR sequence (e.g., orthologous human TTR sequence comprising a V30M mutation). As one example, the replaced region of the endogenous TTR locus can comprise both a coding sequence (i.e., all or part of an exon) and a non-coding sequence (i.e., all or part of intron), such as at least one exon and at least one intron. For example, the replaced region of the endogenous TTR locus can comprise both an exonic sequence (i.e., all or part of an exon) and an intronic sequence (i.e., all or part of intron), such as at least one exon and at least one intron. For example, the replaced region can comprise at least one exon and at least one intron. The replaced region comprising both coding sequence and non-coding sequence (e.g., comprising both exonic sequence and intronic sequence) can be a contiguous region of the endogenous TTR locus, meaning there is no intervening sequence between the replaced coding sequence and the replaced non-coding sequence (e.g., between the replaced exonic sequence and the replaced intronic sequence). For example, the replaced region can comprise at least one exon and at least one adjacent intron. The replaced region can comprise one exon, two exons, three exons, or all four exons of the endogenous TTR locus. The inserted human TTR sequence can comprise one exon, two exons, three exons, or all four exons of a human TTR gene. Likewise, the replaced region can comprise one intron, two introns, or all three introns of the endogenous TTR locus. The inserted human TTR sequence can comprise one intron, two introns, or all three introns of a human TTR gene. Optionally, one or more introns and/or one or more exons of the endogenous TTR locus remain unmodified (i.e., not deleted and replaced). For example, the first exon of the endogenous TTR locus can remain unmodified. Similarly, the first exon and the first intron of the endogenous TTR locus can remain unmodified.

In one specific example, the entire coding sequence for the transthyretin precursor protein can be deleted and replaced with the orthologous human TTR sequence. For example, the region of the endogenous TTR locus beginning at the start codon and ending at the stop codon can be deleted and replaced with the orthologous human TTR sequence.

Flanking untranslated regions including regulatory sequences can also be humanized or remain endogenous. The first exon of a TTR locus typically includes a 5′ untranslated region (UTR) upstream of the start codon. Likewise, the last exon (fourth exon) of a TTR locus typically includes a 3′ UTR downstream of the stop codon. Regions upstream of the TTR start codon and downstream of the TTR stop codon can either be unmodified or can be deleted and replaced with the orthologous human TTR sequence. For example, the 5′ UTR, the 3′UTR, or both the 5′ UTR and the 3′ UTR can be humanized, or the 5′ UTR, the 3′UTR, or both the 5′ UTR and the 3′ UTR can remain endogenous. One or both of the human 5′ and 3′ UTRs can be inserted, and/or one or both of the endogenous 5′ and 3′ UTRs can be deleted. In one specific example, the 5′ UTR remains endogenous. In another specific example, the 3′ UTR is humanized, but the 5′ UTR remains endogenous. In another specific example, the 5′ UTR remains endogenous, and a human TTR 3′ UTR is inserted into the endogenous TTR locus. For example, the human TTR 3′ UTR can replace the endogenous 3′ UTR or can be inserted without replacing the endogenous 3′ UTR (e.g., it can be inserted upstream of the endogenous 3′ UTR). For example, the endogenous 5′ UTR (or a portion thereof) and the endogenous 3′ UTR (or a portion thereof) can remain at the humanized TTR locus, and the human 3′ UTR (or a portion thereof) can be inserted upstream of the endogenous 3′ UTR. Depending on the extent of replacement by orthologous sequences, regulatory sequences, such as a promoter, can be endogenous or supplied by the replacing human orthologous sequence. For example, the humanized TTR locus can include the endogenous non-human animal TTR promoter (i.e., the inserted human TTR sequence or humanized TTR-coding sequence can be operably linked to the endogenous non-human animal TTR promoter).

One or more or all of the regions encoding the signal peptide and the mature transthyretin protein (i.e., after removal of the signal peptide from the transthyretin precursor protein) or one or more of such regions can remain endogenous. Exemplary coding sequences for a mouse transthyretin signal peptide and mature transthyretin protein are set forth in SEQ ID NOS: 17 and 18, respectively. Exemplary coding sequences for a human transthyretin signal peptide and mature transthyretin protein are set forth in SEQ ID NOS: 8 and 9, respectively. An exemplary coding sequence for a human mature transthyretin protein comprising a V30M mutation is set forth in SEQ ID NO: 10.

For example, all or part of the region of the TTR locus encoding the signal peptide can be humanized, and/or all or part of the region of the TTR locus encoding the mature transthyretin protein can be humanized. In one example, all or part of the region of the TTR locus encoding the signal peptide is humanized. Optionally, the CDS of the human transthyretin signal peptide comprises, consists essentially of, or consists of a sequence that is at least about 85%, at least about 90%, at least about 95%, at least about 96%, at least about 97%, at least about 98%, at least about 99%, or about 100% identical to SEQ ID NO: 8 (or degenerates thereof). The humanized transthyretin precursor protein is expressed and can retain the activity of the native transthyretin precursor protein and/or the human transthyretin precursor protein. In another example, all or part of the region of the TTR locus encoding the mature transthyretin protein is humanized. Optionally, the CDS of the human mature transthyretin protein comprising a V30M mutation comprises, consists essentially of, or consists of a sequence that is at least about 85%, at least about 90%, at least about 95%, at least about 96%, at least about 97%, at least about 98%, at least about 99%, or about 100% identical to SEQ ID NO: 10 (or degenerates thereof). The humanized transthyretin protein is expressed and can retain the activity of the native transthyretin protein and/or the human transthyretin protein. In another example, all or part of the region of the TTR locus encoding the signal peptide and the mature transthyretin protein is humanized. The humanized transthyretin protein can retain the activity of the native transthyretin protein and/or the human transthyretin protein. For example, the region of the TTR locus encoding all of the signal peptide and the mature transthyretin precursor protein can be humanized such that a fully humanized transthyretin precursor protein is produced with a human signal peptide and a human mature transthyretin protein region.

One or more of the regions encoding the signal peptide and the mature transthyretin protein region can remain endogenous. For example, the region encoding the signal peptide can remain endogenous. Optionally, the CDS of the endogenous transthyretin signal peptide comprises, consists essentially of, or consists of a sequence that is at least about 85%, at least about 90%, at least about 95%, at least about 96%, at least about 97%, at least about 98%, at least about 99%, or about 100% identical to SEQ ID NO: 17 (or degenerates thereof). For example, the region encoding the mature transthyretin protein can remain endogenous. Optionally, the CDS of the endogenous mature transthyretin protein comprises, consists essentially of, or consists of a sequence that is at least about 85%, at least about 90%, at least about 95%, at least about 96%, at least about 97%, at least about 98%, at least about 99%, or about 100% identical to SEQ ID NO: 18 (or degenerates thereof) but comprising a V30M mutation. In each case, the transthyretin precursor protein is expressed and can retain the activity of the native transthyretin precursor protein and/or the human transthyretin precursor protein.

The transthyretin precursor protein encoded by the humanized TTR locus can comprise one or more domains that are from a human transthyretin precursor protein and/or one or more domains that are from an endogenous (i.e., native) transthyretin precursor protein. Exemplary amino acid sequences for a mouse transthyretin signal peptide and mature transthyretin protein are set forth in SEQ ID NOS: 14 and 15, respectively. Exemplary amino acid sequences for a human transthyretin signal peptide and mature transthyretin protein are set forth in SEQ ID NOS: 3 and 4, respectively. An exemplary amino acid sequence for a human mature transthyretin protein comprising a V30M mutation is set forth in SEQ ID NO: 5.

The humanized transthyretin precursor protein can comprise one or more or all of a human transthyretin signal peptide and a human mature transthyretin protein region. As one example, the humanized transthyretin precursor protein can comprise a human transthyretin signal peptide and a human mature transthyretin protein region.

The humanized transthyretin precursor protein encoded by the humanized TTR locus can also comprise one or more domains that are from the endogenous (i.e., native) non-human animal transthyretin precursor protein. As one example, the transthyretin precursor protein encoded by the humanized TTR locus can comprise a signal peptide from the endogenous (i.e., native) non-human animal transthyretin precursor protein. For example, the humanized transthyretin precursor protein can comprise an endogenous transthyretin signal peptide and a human mature transthyretin protein region.

Domains in a humanized transthyretin precursor protein that are from a human transthyretin precursor protein can be encoded by a fully humanized sequence (i.e., the entire sequence encoding that domain is replaced with the orthologous human TTR sequence) or can be encoded by a partially humanized sequence (i.e., some of the sequence encoding that domain is replaced with the orthologous human TTR sequence, and the remaining endogenous (i.e., native) sequence encoding that domain encodes the same amino acids as the orthologous human TTR sequence such that the encoded domain is identical to that domain in the human transthyretin precursor protein). For example, part of the region of the TTR locus encoding the signal peptide (e.g., encoding the N-terminal region of the signal peptide) can remain endogenous TTR sequence, wherein the amino acid sequence of the region of the signal peptide encoded by the remaining endogenous TTR sequence is identical to the corresponding orthologous human transthyretin precursor protein amino acid sequence. As another example, part of the region of the TTR locus encoding the mature transthyretin protein (e.g., encoding the C-terminal region of the mature transthyretin protein) can remain endogenous TTR sequence, wherein the amino acid sequence of the region of the mature transthyretin protein encoded by the remaining endogenous TTR sequence is identical to the corresponding orthologous human mature transthyretin protein amino acid sequence.

Likewise, domains in a humanized protein that are from the endogenous transthyretin precursor protein cay be encoded by a fully endogenous sequence (i.e., the entire sequence encoding that domain is the endogenous TTR sequence) or can be encoded by a partially humanized sequence (i.e., some of the sequence encoding that domain is replaced with the orthologous human TTR sequence, but the orthologous human TTR sequence encodes the same amino acids as the replaced endogenous TTR sequence such that the encoded domain is identical to that domain in the endogenous transthyretin precursor protein). For example, part of the region of the TTR locus encoding the signal peptide (e.g., encoding the C-terminal region of the signal peptide) can be replaced with orthologous human TTR sequence, wherein the amino acid sequence of the region of the signal peptide encoded by the orthologous human TTR sequence is identical to the corresponding endogenous amino acid sequence. As another example, part of the region of the TTR locus encoding the mature transthyretin protein (e.g., encoding the N-terminal region of the mature transthyretin protein) can be replaced with orthologous human TTR sequence, wherein the amino acid sequence of the region of the mature transthyretin protein encoded by the orthologous human TTR sequence is identical to the corresponding endogenous amino acid sequence.

As one example, the transthyretin precursor protein encoded by the humanized TTR locus can comprise an endogenous transthyretin precursor signal peptide. Optionally, the endogenous transthyretin precursor signal peptide comprises, consists essentially of, or consists of a sequence that is at least about 85%, at least about 90%, at least about 95%, at least about 96%, at least about 97%, at least about 98%, at least about 99%, or about 100% identical to SEQ ID NO: 14. The humanized transthyretin precursor protein is expressed and can retain the activity of the native transthyretin precursor protein and/or the human transthyretin precursor protein. As another example, the transthyretin precursor protein encoded by the humanized TTR locus can comprise an endogenous mature transthyretin protein region. Optionally, the endogenous mature transthyretin protein comprises, consists essentially of, or consists of a sequence that is at least about 85%, at least about 90%, at least about 95%, at least about 96%, at least about 97%, at least about 98%, at least about 99%, or about 100% identical to SEQ ID NO: 15, but having a V30M mutation. The humanized transthyretin precursor protein is expressed and can retain the activity of the native transthyretin precursor protein and/or the human transthyretin precursor protein. As one example, the transthyretin precursor protein encoded by the humanized TTR locus can comprise a human transthyretin precursor signal peptide. Optionally, the human transthyretin precursor signal peptide comprises, consists essentially of, or consists of a sequence that is at least about 85%, at least about 90%, at least about 95%, at least about 96%, at least about 97%, at least about 98%, at least about 99%, or about 100% identical to SEQ ID NO: 3. The humanized transthyretin precursor protein is expressed and can retain the activity of the native transthyretin precursor protein and/or the human transthyretin precursor protein. As another example, the transthyretin precursor protein encoded by the humanized TTR locus can comprise a human mature transthyretin protein region. Optionally, the human mature transthyretin protein comprising the V30M mutation comprises, consists essentially of, or consists of a sequence that is at least about 85%, at least about 90%, at least about 95%, at least about 96%, at least about 97%, at least about 98%, at least about 99%, or about 100% identical to SEQ ID NO: 5. The humanized transthyretin precursor protein is expressed and can retain the activity of the native transthyretin precursor protein and/or the human transthyretin precursor protein. For example, the transthyretin precursor protein encoded by the humanized TTR locus comprising the V30M mutation can comprise, consist essentially of, or consist of a sequence that is at least about 85%, at least about 90%, at least about 95%, at least about 96%, at least about 97%, at least about 98%, at least about 99%, or about 100% identical to SEQ ID NO: 2. Optionally, the TTR CDS encoded by the humanized TTR locus comprising the V30M mutation can comprise, consist essentially of, or consist of a sequence that is at least about 85%, at least about 90%, at least about 95%, at least about 96%, at least about 97%, at least about 98%, at least about 99%, or about 100% identical to SEQ ID NO: 7 (or degenerates thereof). In each case, the humanized transthyretin precursor protein is expressed and can retain the activity of the native transthyretin precursor protein and/or the human transthyretin precursor protein.

Optionally, a humanized TTR locus can comprise other elements. Examples of such elements can include selection cassettes, reporter genes, recombinase recognition sites, or other elements. Alternatively, the humanized TTR locus can lack other elements (e.g., can lack a selection marker or selection cassette). Examples of suitable reporter genes and reporter proteins are disclosed elsewhere herein. Examples of suitable selection markers include neomycin phosphotransferase (neoᵣ), hygromycin B phosphotransferase (hygᵣ), puromycin-N-acetyltransferase (puroᵣ), blasticidin S deaminase (bsrᵣ), xanthine/guanine phosphoribosyl transferase (gpt), and herpes simplex virus thymidine kinase (HSV-k). Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

Other elements such as reporter genes or selection cassettes can be self-deleting cassettes flanked by recombinase recognition sites. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. The polynucleotide encoding the selection marker can be operably linked to a promoter active in a cell being targeted. Examples of promoters are described elsewhere herein. As another specific example, a self-deleting selection cassette can comprise a hygromycin resistance gene coding sequence operably linked to one or more promoters (e.g., both human ubiquitin and EM7 promoters) followed by a polyadenylation signal, followed by a Crei coding sequence operably linked to one or more promoters (e.g., an mPrm1 promoter), followed by another polyadenylation signal, wherein the entire cassette is flanked by loxP sites.

The humanized TTR locus can also be a conditional allele. For example, the conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

As a specific example, the humanized TTR locus comprising the V30M mutation can be one in which the region of the endogenous TTR locus being deleted and/or replaced with the orthologous human TTR sequence comprises, consists essentially of, or consists of the region from the TTR start codon to the stop codon. The human TTR sequence being inserted can further comprise a human TTR 3′ UTR. For example, the endogenous TTR sequence deleted from the humanized TTR locus can comprise, consist essentially of, or consist of the region from the endogenous TTR start codon to the endogenous TTR stop codon, and/or the human TTR sequence at the humanized TTR locus comprising the V30M mutation can comprise, consist essentially of, or consist of the region from the TTR start codon to the end of the 3′ UTR. Optionally, the TTR coding sequence in the modified endogenous TTR locus is operably linked to the endogenous TTR promoter.

In one specific example, the human TTR sequence at the humanized endogenous TTR locus comprising the V30M mutation can comprise, consist essentially of, or consist of a sequence at least about 85%, at least about 90%, at least about 95%, at least about 96%, at least about 97%, at least about 98%, at least about 99%, or about 100% identical to the sequence set forth in SEQ ID NO: 24. In another specific example, the humanized TTR locus can encode a protein (e.g., transthyretin precursor protein comprising a V30M mutation) comprising, consisting essentially of, or consisting of a sequence at least about 85%, at least about 90%, at least about 95%, at least about 96%, at least about 97%, at least about 98%, at least about 99%, or about 100% identical to the sequence set forth in SEQ ID NO: 2 or can encode a mature transthyretin protein (comprising a V30M mutation) comprising, consisting essentially of, or consisting of a sequence at least about 85%, at least about 90%, at least about 95%, at least about 96%, at least about 97%, at least about 98%, at least about 99%, or about 100% identical to the sequence set forth in SEQ ID NO: 5. In another specific example, the humanized TTR locus can comprise a coding sequence (e.g., coding sequence for a transthyretin precursor protein comprising a V30M mutation) comprising, consisting essentially of, or consisting of a sequence at least about 85%, at least about 90%, at least about 95%, at least about 96%, at least about 97%, at least about 98%, at least about 99%, or about 100% identical to the sequence set forth in SEQ ID NO: 7 or can comprise a coding sequence for a mature transthyretin protein (comprising a V30M mutation) comprising, consisting essentially of, or consisting of a sequence at least about 85%, at least about 90%, at least about 95%, at least about 96%, at least about 97%, at least about 98%, at least about 99%, or about 100% identical to the sequence set forth in SEQ ID NO: 10. In another specific example, the humanized TTR locus comprising the V30M mutation can comprise, consist essentially of, or consist of a sequence at least about 85%, at least about 90%, at least about 95%, at least about 96%, at least about 97%, at least about 98%, at least about 99%, or about 100% identical to the sequence set forth in SEQ ID NO: 22 or 23.

A control non-human animal comprising a humanized TTR wild type locus can also be generated. The coding sequence (CDS) at the humanized TTR wild type locus can comprise, consist essentially of, or consist of a sequence that is at least about 85%, at least about 90%, at least about 95%, at least about 96%, at least about 97%, at least about 98%, at least about 99%, or about 100% identical to SEQ ID NO: 6 (or degenerates thereof that encode the same protein). The resulting human transthyretin precursor protein encoded by the humanized TTR wild type locus can comprise, consist essentially of, or consist of a sequence that is at least about 85%, at least about 90%, at least about 95%, at least about 96%, at least about 97%, at least about 98%, at least about 99%, or about 100% identical to SEQ ID NO: 1.

As another specific example, the humanized TTR locus can be one in which the region of the endogenous TTR locus being deleted and/or replaced with the orthologous human TTR sequence comprises, consists essentially of, or consists of the region from the start of the second TTR exon to the stop codon. The human TTR sequence being inserted can further comprise a human TTR 3′ UTR. For example, the human TTR sequence at the humanized TTR locus can comprise, consist essentially of, or consist of the region from the start of the second human TTR exon to the end of the 3′ UTR. Optionally, the TTR coding sequence in the modified endogenous Ttr locus is operably linked to the endogenous TTR promoter.

TTR protein expressed from a humanized TTR locus can be an entirely human TTR protein or a chimeric endogenous/human TTR protein (e.g., if the non-human animal is a mouse, a chimeric mouse/human TTR protein). For example, the signal peptide of the transthyretin precursor protein can be endogenous, and the remainder of the protein can be human. Alternatively, the N-terminus of the transthyretin precursor protein can be endogenous, and the remainder of the protein can be human. For example, the N-terminal 5, 6, 7, 8, 9, 10, 11, 12, 13, 14, 15, 16, 17, 18, 19, 20, 21, 22, 23, 24, or 25 amino acids can be endogenous, and the remainder can be human. In a specific example, the 23 amino acids at the N-terminus are endogenous, and the remainder of the protein is human.

C. Non-Human Animal Genomes, Non-Human Animal Cells, and Non-Human Animals Comprising a Humanized TTR Locus Comprising a V30M Mutation

Non-human animal genomes, non-human animal cells, and non-human animals comprising a humanized TTR locus as described elsewhere herein are provided. The genomes, cells, or non-human animals can express a humanized TTR protein encoded by the humanized TTR locus. The genomes, cells, or non-human animals can be male or female. The genomes, cells, or non-human animals can be heterozygous or homozygous for the humanized TTR locus. Non-human animal genomes, non-human animal cells, and non-human animals comprising a humanized TTR locus as described elsewhere herein and CRISPR/Cas synergistic activation mediator system components are also provided. The genomes, cells, or non-human animals can be heterozygous or homozygous for the humanized TTR locus, and they can be heterozygous or homozygous for CRISPR/Cas synergistic activation mediator system components. A diploid organism has two alleles at each genetic locus. Each pair of alleles represents the genotype of a specific genetic locus. Genotypes are described as homozygous if there are two identical alleles at a particular locus and as heterozygous if the two alleles differ. A non-human animal comprising a humanized TTR locus can comprise the humanized TTR locus in its germline. Likewise, a non-human animal comprising CRISPR/Cas synergistic activation mediator system components can comprise the CRISPR/Cas synergistic activation mediator system components in its germline.

The non-human animal genomes or cells provided herein can be, for example, any non-human animal genome or cell comprising a TTR locus or a genomic locus homologous or orthologous to the human TTR locus. The genomes can be from or the cells can be eukaryotic cells, which include, for example, animal cells, mammalian cells, non-human mammalian cells, and human cells. The term “animal” includes any member of the animal kingdom, including, for example, mammals, fishes, reptiles, amphibians, birds, and worms. A mammalian cell can be, for example, a non-human mammalian cell, a rodent cell, a rat cell, or a mouse cell. Other non-human mammals include, for example, non-human primates. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell (e.g., a non-ES cell). Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatoblasts or hepatocytes.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, hepatocytes.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. A specific example of an immortalized cell line is the HepG2 human liver cancer cell line. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a humanized TTR locus comprising a V30M mutation as described herein can be made by the methods described elsewhere herein. Likewise, non-human animals comprising a humanized TTR locus comprising a V30M mutation and CRISPR/Cas synergistic activation mediator system components as described herein can be made by the methods described elsewhere herein. The term “animal” includes any member of the animal kingdom, including, for example, mammals, fishes, reptiles, amphibians, birds, and worms. In a specific example, the non-human animal is a non-human mammal. Non-human mammals include, for example, non-human primates and rodents (e.g., mice and rats). The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

Non-human animals comprising a humanized TTR locus comprising a V30M mutation can express the humanized TTR protein at any level. For example, non-human animals comprising a humanized TTR locus can express humanized TTR protein at levels of at least about 1, at least about 2, at least about 3, at least about 4, at least about 5, at least about 6, at least about 7, at least about 8, at least about 9, at least about 10, at least about 12, at least about 14, at least about 15, at least about 16, at least about 18, at least about 20, at least about 22, at least about 24, at least about 25, at least about 26, at least about 28, or at least about 30 μg/mL in the serum. Likewise, non-human animals comprising a humanized TTR locus comprising a V30M mutation and CRISPR/Cas synergistic activation mediator system components can express TTR protein encoded by the humanized TTR locus (e.g., human TTR) at any level (e.g., without SAM guide RNAs targeting the humanized TTR locus or with SAM guide RNAs targeting the humanized TTR locus). For example, serum levels of TTR protein encoded by the humanized TTR locus (e.g., human TTR) can be about the same as physiological levels in a human, which are well-known. In one example, serum levels of TTR protein encoded by the humanized TTR locus (e.g., human TTR) can be at least about 10 μg/mL, at least about 20 μg/mL, at least about 30 μg/mL, at least about 40 μg/mL, at least about 50 μg/mL, at least about 60 μg/mL, at least about 70 μg/mL, at least about 80 μg/mL, at least about 90 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/mL, at least about 500 μg/mL, at least about 600 μg/mL, at least about 700 μg/mL, at least about 800 μg/mL, at least about 900 μg/mL, or at least about 1000 μg/mL.

In another example, serum levels of TTR protein encoded by the humanized TTR locus (e.g., human TTR) can be between about 10 μg/mL and about 20 μg/mL, between about 20 μg/mL and about 30 μg/mL, between about 30 μg/mL and about 40 μg/mL, between about 40 μg/mL and about 50 μg/mL, between about 50 μg/mL and about 60 μg/mL, between about 60 μg/mL and about 70 μg/mL, between about 70 μg/mL and about 80 μg/mL, between about 80 μg/mL and about 90 μg/mL, between about 90 μg/mL and about 100 μg/mL, between about 100 μg/mL and about 150 μg/mL, between about 150 μg/mL and about 200 μg/mL, between about 200 μg/mL and about 250 μg/mL, between about 250 μg/mL and about 300 μg/mL, between about 300 μg/mL and about 350 μg/mL, between about 350 μg/mL and about 400 μg/mL, between about 400 μg/mL and about 450 μg/mL, between about 450 μg/mL and about 500 μg/mL, between about 500 μg/mL and about 600 μg/mL, between about 600 μg/mL and about 700 μg/mL, between about 700 μg/mL and about 800 μg/mL, between about 800 μg/mL and about 900 μg/mL, or between about 900 μg/mL and about 1000 μg/mL.

In another example, serum levels of TTR protein encoded by the humanized TTR locus (e.g., human TTR) can be between about 10 μg/mL and about 20 μg/mL, between about 10 μg/mL and about 30 μg/mL, between about 10 μg/mL and about 40 μg/mL, between about 10 μg/mL and about 50 μg/mL, between about 10 μg/mL and about 60 μg/mL, between about 10 μg/mL and about 70 μg/mL, between about 10 μg/mL and about 80 μg/mL, between about 10 μg/mL and about 90 μg/mL, between about 10 μg/mL and about 100 μg/mL, between about 10 μg/mL and about 150 μg/mL, between about 10 μg/mL and about 200 μg/mL, between about 10 μg/mL and about 250 μg/mL, between about 10 μg/mL and about 300 μg/mL, between about 10 μg/mL and about 350 μg/mL, between about 10 μg/mL and about 400 μg/mL, between about 10 μg/mL and about 450 μg/mL, between about 10 μg/mL and about 500 μg/mL, between about 10 μg/mL and about 600 μg/mL, between about 10 μg/mL and about 700 μg/mL, between about 10 μg/mL and about 800 μg/mL, between about 10 μg/mL and about 900 μg/mL, or between about 10 μg/mL and about 1000 μg/mL.

In another example, serum levels of TTR protein encoded by the humanized TTR locus (e.g., human TTR) can be between about 10 μg/mL and about 1000 μg/mL, between about 20 μg/mL and about 1000 μg/mL, between about 30 μg/mL and about 1000 μg/mL, between about 40 μg/mL and about 1000 μg/mL, between about 50 μg/mL and about 1000 μg/mL, between about 60 μg/mL and about 1000 μg/mL, between about 70 μg/mL and about 1000 μg/mL, between about 80 μg/mL and about 1000 μg/mL, between about 90 μg/mL and about 1000 μg/mL, between about 100 μg/mL and about 1000 μg/mL, between about 150 μg/mL and about 1000 μg/mL, between about 200 μg/mL and about 1000 μg/mL, between about 250 μg/mL and about 1000 μg/mL, between about 300 μg/mL and about 1000 μg/mL, between about 350 μg/mL and about 1000 μg/mL, between about 400 μg/mL and about 1000 μg/mL, between about 450 μg/mL and about 1000 μg/mL, between about 500 μg/mL and about 1000 μg/mL, between about 600 μg/mL and about 1000 μg/mL, between about 700 μg/mL and about 1000 μg/mL, between about 800 μg/mL and about 1000 μg/mL, or between about 900 μg/mL and about 1000 μg/mL.

In another example, serum levels of TTR protein encoded by the humanized TTR locus (e.g., human TTR) can be between about 10 μg/mL and about 450 μg/mL, between about 50 μg/mL and about 400 μg/mL, between about 100 μg/mL and about 350 μg/mL, between about 150 μg/mL and about 300 μg/mL, or between about 200 μg/mL and about 250 μg/mL.

Non-human animals comprising a humanized TTR locus comprising a V30M locus can also comprise TTR amyloid deposition or the presence of TTR aggregates or fibrils or phenotypes such as neuropathy or peripheral neuropathy or TTR amyloid neuropathy or polyneuropathy (e.g., TTR amyloid deposits around peripheral nerves). The protein deposits can occur, e.g., in the peripheral nervous system, which is made up of nerves connecting the brain and spinal cord to muscles and sensory cells that detect sensations such as touch, pain, heat, and sound. Protein deposits in these nerves can result in a loss of sensation in the extremities (peripheral neuropathy). The autonomic nervous system, which controls involuntary body functions such as blood pressure, heart rate, and digestion, may also be affected by amyloidosis. In some cases, the brain and spinal cord (central nervous system) are affected. Other areas of amyloidosis include the heart, kidneys, eyes, and gastrointestinal tract.

D. Seeded Non-Human Animal Cells, and Non-Human Animals Comprising a Humanized TTR Locus Comprising a V30M Mutation

Non-human animal cells and non-human animals comprising a humanized TTR locus can be seeded with pre-formed TTR aggregates or fibrils (i.e., exogenous TTR aggregates or fibrils). The pre-formed TTR aggregates or fibrils can be V30M TTR aggregates or fibrils, can be wild type TTR aggregates or fibrils, or can be TTR aggregates or fibrils in which the TTR comprises a mutation other than or in addition to V30M. Likewise, the TTR aggregates or fibrils can be human TTR aggregates or fibrils (e.g., human TTR V30M aggregates or fibrils) or can be mouse TTR aggregates or fibrils. In non-human animals comprising a humanized TTR locus, the pre-formed TTR aggregates or fibrils can be injected via intravenous injection (e.g., tail vein injection). For example, the pre-formed TTR aggregate or fibrils can be administered via hydrodynamic delivery. In some cases, the TTR aggregates or fibrils can be administered together with heparin (i.e., exogenous heparin), which can serve as a template for amyloid fibrils to form and accelerate TTR amyloid deposition. The non-human animals can comprise the pre-formed TTR aggregates or fibrils in the liver (i.e., the liver can be a site of exogenous TTR deposition in the non-human animals). Likewise, the non-human animals can comprise the pre-formed TTR aggregates or fibrils in the lung, the heart, the spleen, the kidney, and/or other organs (i.e., these organs can be sites of exogenous TTR deposition in the non-human animals).

### III. Non-Human Animals Comprising Synergistic Activation Mediator (SAM) Expression Cassettes

The non-human animal genomes, non-human animal cells, and non-human animals disclosed herein also comprise Clustered Regularly Interspersed Short Palindromic Repeats (CRISPR)/CRISPR-associated (Cas)-based synergistic activation mediator (SAM) expression cassettes for use in methods of activating transcription of target genes such as the humanized TTR genes disclosed herein in vitro, ex vivo, or in vivo. The SAM systems described herein comprise chimeric Cas proteins and chimeric adaptor proteins and can be used with guide RNAs as described elsewhere herein to activate transcription of target genes such as the humanized TTR genes disclosed herein. The guide RNAs can be encoded by genomically integrated expression cassettes, or they can be provided by AAV or any other suitable means. Chimeric Cas proteins (e.g., chimeric Cas proteins, such as chimeric Cas9 proteins, such as a chimeric Streptococcus pyogenes Cas9 protein, a chimeric Campylobacter jejuni Cas9 protein, or a chimeric Staphylococcus aureus Cas9 protein) and chimeric adaptor proteins (e.g., comprising an adaptor protein that specifically binds to an adaptor-binding element within a guide RNA; and one or more heterologous transcriptional activation domains) are described in further detail elsewhere herein.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). CRISPR/Cas systems used in the compositions and methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

The methods and compositions disclosed herein employ the CRISPR/Cas systems by using or testing the ability of CRISPR complexes (comprising a guide RNA (gRNA) complexed with a chimeric Cas protein and a chimeric adaptor protein) to induce transcriptional activation of a target genomic locus in vivo.

The genomes, cells, and non-human animals disclosed herein comprise a chimeric Cas protein expression cassette and/or a chimeric adaptor protein expression cassette. For example, the genomes, cells, and non-human animals disclosed herein can comprise a synergistic activation mediator (SAM) expression cassette comprising a chimeric Cas protein coding sequence and a chimeric adaptor protein coding sequence.

Such genomes, cells, or non-human animals comprising a SAM expression cassette have the advantage of needing delivery only of guide RNAs in order to induce transcriptional activation of a target genomic locus. Some such genomes, cells, or non-human animals also comprise a guide RNA expression cassette so that all components required for transcriptional activation of a target gene are already present. The SAM systems can be used in such cells to provide increased expression of target genes in any desired manner. For example, expression of one or more target genes can be increased in a constitutive manner or in a regulated manner (e.g., inducible, tissue-specific, temporally regulated, and so forth).

A. Chimeric Cas Proteins

Provided are chimeric Cas proteins that can bind to the guide RNAs disclosed elsewhere herein to activate transcription of target genes. Such chimeric Cas proteins can comprise: (a) a DNA-binding domain that is a Clustered Regularly Interspaced Short Palindromic Repeats (CRISPR)-associated (Cas) protein or a functional fragment or variant thereof that is capable of forming a complex with a guide RNA and binding to a target sequence; and (b) one or more transcriptional activation domains or functional fragments or variants thereof. For example, such fusion proteins can comprise 1, 2, 3, 4, 5, or more transcriptional activation domains (e.g., two or more heterologous transcriptional activation domains or three or more heterologous transcriptional activation domains). In one example, the chimeric Cas protein can comprise a catalytically inactive Cas protein (e.g., dCas9) and a VP64 transcriptional activation domain or a functional fragment or variant thereof. For example, such a chimeric Cas protein can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the dCas9-VP64 chimeric Cas protein sequence set forth in SEQ ID NO: 97. However, chimeric Cas proteins in which the transcriptional activation domains comprise other transcriptional activation domains or functional fragments or variants thereof and/or in which the Cas protein comprises other Cas proteins (e.g., catalytically inactive Cas proteins) are also provided. Examples of other suitable transcriptional activation domains are provided elsewhere herein.

The transcriptional activation domain(s) can be located at the N-terminus, the C-terminus, or anywhere within the Cas protein. For example, the transcriptional activation domain(s) can be attached to the Rec1 domain, the Rec2 domain, the HNH domain, or the PI domain of a Streptococcus pyogenes Cas9 protein or any corresponding region of an orthologous Cas9 protein or homologous or orthologous Cas protein when optimally aligned with the S. pyogenes Cas9 protein. For example, the transcriptional activation domain can be attached to the Rec1 domain at position 553, the Rec1 domain at position 575, the Rec2 domain at any position within positions 175-306 or replacing part of or the entire region within positions 175-306, the HNH domain at any position within positions 715-901 or replacing part of or the entire region within positions 715-901, or the PI domain at position 1153 of the S. pyogenes Cas9 protein. See, e.g., WO 2016/049258, herein incorporated by reference in its entirety for all purposes. The transcriptional activation domain may be flanked by one or more linkers on one or both sides as described elsewhere herein.

Chimeric Cas proteins can also be operably linked or fused to additional heterologous polypeptides. The fused or linked heterologous polypeptide can be located at the N-terminus, the C-terminus, or anywhere internally within the chimeric Cas protein. For example, a chimeric Cas protein can further comprise a nuclear localization signal. Examples of suitable nuclear localization signals and other modifications to Cas proteins are described in further detail elsewhere herein.

(1) Cas Proteins

Cas proteins generally comprise at least one RNA recognition or binding domain that can interact with guide RNAs. A functional fragment or functional variant of a Cas protein is one that retains the ability to form a complex with a guide RNA and to bind to a target sequence in a target gene (and, for example, activate transcription of the target gene).

In addition to transcriptional activation domain as described elsewhere herein, Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus. In one example, the Cas protein portions of the chimeric Cas proteins disclosed herein have been modified to have decreased nuclease activity (e.g., nuclease activity is diminished by at least about 70%, at least about 75%, at least about 80%, at least about 85%, at least about 90%, at least about 95%, at least about 96%, at least about 97%, at least about 98%, at least about 99%, or 100% compared to a wild type Cas protein) or to lack substantially all nuclease activity (i.e., nuclease activity is diminished by at least 90%, at least 95%, at least 97%, at least 98%, at least 99%, or 100% compared to a wild type Cas protein, or having no more than about 0%, no more than about 1%, no more than about 2%, no more than about 3%, no more than about 5%, or no more than about 10% of the nuclease activity of a wild type Cas protein). A nuclease-inactive Cas protein is a Cas protein having mutations known to be inactivating mutations in its catalytic (i.e., nuclease) domains (e.g., inactivating mutations in a RuvC-like endonuclease domain in a Cpf1 protein, or inactivating mutations in both an HNH endonuclease domain and a RuvC-like endonuclease domain in Cas9) or a Cas protein having nuclease activity diminished by at least about 97%, least about 98%, least about 99%, or 100% compared to a wild type Cas protein. Examples of different Cas protein mutations to reduce or substantially eliminate nuclease activity are disclosed below.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (assigned SwissProt accession number Q99ZW2) is an exemplary Cas9 protein. Cas9 from S. aureus (SaCas9) (assigned UniProt accession number J7RUA5) is another exemplary Cas9 protein. Cas9 from Campylobacter jejuni (CjCas9) (assigned UniProt accession number Q0P897) is another exemplary Cas9 protein. See, e.g., Kim et al. (2017) Nat. Comm. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, at least 85%, at least 90%, at least 91%, at least 92%, at least 93%, at least 94%, at least 95%, at least 96%, at least 97%, at least 98%, at least 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9. One example of a catalytically inactive Cas9 protein (dCas9) comprises, consists essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the dCas9 protein sequence set forth in SEQ ID NO: 98.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) and a substitution at position D10 (e.g., D10A substitution) to generate a nuclease-inactive Cas protein. See, e.g., WO 2016/106236, herein incorporated by reference in its entirety for all purposes. Examples of inactivating mutations in the catalytic domains of Nme2Cas9 are also known (e.g., combination of D16A and H588A). Examples of inactivating mutations in the catalytic domains of St1Cas9 are also known (e.g., combination of D9A, D598A, H599A, and N622A). Examples of inactivating mutations in the catalytic domains of St3Cas9 are also known (e.g., combination of D10A and N870A). Examples of inactivating mutations in the catalytic domains of CjCas9 are also known (e.g., combination of D8A and H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Cas proteins can also be operably linked to heterologous polypeptides as fusion proteins. For example, in addition to transcriptional activation domains, a Cas protein can be fused to a cleavage domain or an epigenetic modification domain. See WO 2014/089290, herein incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification) or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

(2) Transcriptional Activation Domains

The chimeric Cas proteins disclosed herein can comprise one or more transcriptional activation domains. Transcriptional activation domains include regions of a naturally occurring transcription factor which, in conjunction with a DNA-binding domain (e.g., a catalytically inactive Cas protein complexed with a guide RNA), can activate transcription from a promoter by contacting transcriptional machinery either directly or through other proteins such as coactivators. Transcriptional activation domains also include functional fragments or variants of such regions of a transcription factor and engineered transcriptional activation domains that are derived from a native, naturally occurring transcriptional activation domain or that are artificially created or synthesized to activate transcription of a target gene. A functional fragment is a fragment that is capable of activating transcription of a target gene when operably linked to a suitable DNA-binding domain. A functional variant is a variant that is capable of activating transcription of a target gene when operably linked to a suitable DNA-binding domain.

A specific transcriptional activation domain for use in the chimeric Cas proteins disclosed herein comprises a VP64 transcriptional activation domain or a functional fragment or variant thereof VP64 is a tetrameric repeat of the minimal activation domain from the herpes simplex VP16 activation domain. For example, the transcriptional activation domain can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the VP64 transcriptional activation domain protein sequence set forth in SEQ ID NO: 99.

Other examples of transcriptional activation domains include herpes simplex virus VP16 transactivation domain, VP64 (quadruple tandem repeat of the herpes simplex virus VP16), a NF-κB p65 (NF-κB trans-activating subunit p65) activation domain, a MyoD1 transactivation domain, an HSF1 transactivation domain (transactivation domain from human heat-shock factor 1), RTA (Epstein Barr virus R transactivator activation domain), a SET7/9 transactivation domain, a p53 activation domain 1, a p53 activation domain 2, a CREB (cAMP response element binding protein) activation domain, an E2A activation domain, an NFAT (nuclear factor of activated T-cells) activation domain, and functional fragments and variants thereof. See, e.g., US 2016/0298125, US 2016/0281072, and WO 2016/049258, each of which is herein incorporated by reference in its entirety for all purposes. Other examples of transcriptional activation domains include Gcn4, MLL, Rtg3, Gln3, Oaf1, Pip2, Pdr1, Pdr3, Pho4, Leu3, and functional fragments and variants thereof. See, e.g., US 2016/0298125, herein incorporated by reference in its entirety for all purposes. Yet other examples of transcriptional activation domains include Spl, Vax, GATA4, and functional fragments and variants thereof. See, e.g., WO 2016/149484, herein incorporated by reference in its entirety for all purposes. Other examples include activation domains from Oct1, Oct-2A, AP-2, CTF1, P300, CBP, PCAF, SRC1, PvALF, ERF-2, OsGAI, HALF-1, C1, AP1, ARF-5, ARF-6, ARF-7, ARF-8, CPRF1, CPRF4, MYC-RP/GP, and TRAB1PC4, and functional fragments and variants thereof. See, e.g., US 2016/0237456, EP3045537, and WO 2011/146121, each of which is incorporated by reference in its entirety for all purposes. Additional suitable transcriptional activation domains are also known. See, e.g., WO 2011/146121, herein incorporated by reference in its entirety for all purposes.

B. Chimeric Adaptor Proteins

Also provided are chimeric adaptor proteins that can bind to the guide RNAs disclosed elsewhere herein. The chimeric adaptor proteins disclosed herein are useful in dCas-synergistic activation mediator (SAM)-like systems to increase the number and diversity of transcriptional activation domains being directed to a target sequence within a target gene to activate transcription of the target gene. Nucleic acids encoding the chimeric adaptor proteins can be genomically integrated in a cell or non-human animal (e.g., a cell or non-human animal comprising a genomically integrated chimeric Cas protein expression cassette) as disclosed elsewhere herein, or the chimeric adaptor proteins or nucleic acids can be introduced into such cells and non-human animals using methods disclosed elsewhere herein (e.g., LNP-mediated delivery or AAV-mediated delivery).

Such chimeric adaptor proteins comprise: (a) an adaptor (i.e., adaptor domain or adaptor protein) that specifically binds to an adaptor-binding element within a guide RNA; and (b) one or more heterologous transcriptional activation domains. For example, such fusion proteins can comprise 1, 2, 3, 4, 5, or more transcriptional activation domains (e.g., two or more heterologous transcriptional activation domains or three or more heterologous transcriptional activation domains). In one example, such chimeric adaptor proteins can comprise: (a) an adaptor (i.e., an adaptor domain or adaptor protein) that specifically binds to an adaptor-binding element in a guide RNA; and (b) two or more transcriptional activation domains. For example, the chimeric adaptor protein can comprise: (a) an MS2 coat protein adaptor that specifically binds to one or more MS2 aptamers in a guide RNA (e.g., two MS2 aptamers in separate locations in a guide RNA); and (b) one or more (e.g., two or more transcriptional activation domains). For example, the two transcriptional activation domains can be p65 and HSF1 transcriptional activation domains or functional fragments or variants thereof. However, chimeric adaptor proteins in which the transcriptional activation domains comprise other transcriptional activation domains or functional fragments or variants thereof are also provided.

The one or more transcriptional activation domains can be fused directly to the adaptor. Alternatively, the one or more transcriptional activation domains can be linked to the adaptor via a linker or a combination of linkers or via one or more additional domains. Likewise, if two or more transcriptional activation domains are present, they can be fused directly to each other or can be linked to each other via a linker or a combination of linkers or via one or more additional domains. Linkers that can be used in these fusion proteins can include any sequence that does not interfere with the function of the fusion proteins. Exemplary linkers are short (e.g., 2-20 amino acids) and are typically flexible (e.g., comprising amino acids with a high degree of freedom such as glycine, alanine, and serine). Some specific examples of linkers comprise one or more units consisting of GGGS (SEQ ID NO: 100) or GGGGS (SEQ ID NO: 101), such as two, three, four, or more repeats of GGGS (SEQ ID NO: 100) or GGGGS (SEQ ID NO: 101) in any combination. Other linker sequences can also be used.

The one or more transcriptional activation domains and the adaptor can be in any order within the chimeric adaptor protein. As one option, the one or more transcriptional activation domains can be C-terminal to the adaptor and the adaptor can be N-terminal to the one or more transcriptional activation domains. For example, the one or more transcriptional activation domains can be at the C-terminus of the chimeric adaptor protein, and the adaptor can be at the N-terminus of the chimeric adaptor protein. However, the one or more transcriptional activation domains can be C-terminal to the adaptor without being at the C-terminus of the chimeric adaptor protein (e.g., if a nuclear localization signal is at the C-terminus of the chimeric adaptor protein). Likewise, the adaptor can be N-terminal to the one or more transcriptional activation domains without being at the N-terminus of the chimeric adaptor protein (e.g., if a nuclear localization signal is at the N-terminus of the chimeric adaptor protein). As another option, the one or more transcriptional activation domains can be N-terminal to the adaptor and the adaptor can be C-terminal to the one or more transcriptional activation domains. For example, the one or more transcriptional activation domains can be at the N-terminus of the chimeric adaptor protein, and the adaptor can be at the C-terminus of the chimeric adaptor protein. As yet another option, if the chimeric adaptor protein comprises two or more transcriptional activation domains, the two or more transcriptional activation domains can flank the adaptor.

Chimeric adaptor proteins can also be operably linked or fused to additional heterologous polypeptides. The fused or linked heterologous polypeptide can be located at the N-terminus, the C-terminus, or anywhere internally within the chimeric adaptor protein. For example, a chimeric adaptor protein can further comprise a nuclear localization signal. A specific example of such a protein comprises an MS2 coat protein (adaptor) linked (either directly or via an NLS) to a p65 transcriptional activation domain C-terminal to the MS2 coat protein (MCP), and HSF1 transcriptional activation domain C-terminal to the p65 transcriptional activation domain. Such a protein can comprise from N-terminus to C-terminus: an MCP; a nuclear localization signal; a p65 transcriptional activation domain; and an HSF1 transcriptional activation domain. For example, a chimeric adaptor protein can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the MCP-p65-HSF1 chimeric adaptor protein sequence set forth in SEQ ID NO: 102.

Chimeric adaptor proteins can also be fused or linked to one or more heterologous polypeptides that provide for subcellular localization. Such heterologous polypeptides can include, for example, one or more nuclear localization signals (NLS) such as the SV40 NLS and/or an alpha-importin NLS for targeting to the nucleus, a mitochondrial localization signal for targeting to the mitochondria, an ER retention signal, and the like. See, e.g., Lange et al. (2007) J. Biol. Chem. 282:5101-5105, herein incorporated by reference in its entirety for all purposes. An NLS can comprise, for example, a stretch of basic amino acids, and can be a monopartite sequence or a bipartite sequence. Optionally, the chimeric adaptor protein comprises two or more NLSs, including an NLS (e.g., an alpha-importin NLS) at the N-terminus and/or an NLS (e.g., an SV40 NLS) at the C-terminus.

Chimeric adaptor proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and W02013/176772, each of which is herein incorporated by reference in its entirety for all purposes. As another example, chimeric adaptor proteins can be fused or linked to a heterologous polypeptide providing increased or decreased stability.

Chimeric adaptor proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Chimeric adaptor proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the chimeric adaptor protein. Likewise, the chimeric adaptor protein can be tethered to the 5′ end, the 3′ end, or to an internal region within the labeled nucleic acid. That is, the labeled nucleic acid can be tethered in any orientation and polarity.

(1) Adaptor Proteins or Adaptor Domains

Adaptors (i.e., adaptor domains or adaptor proteins) are nucleic-acid-binding domains (e.g., DNA-binding domains and/or RNA-binding domains) that specifically recognize and bind to distinct sequences (e.g., bind to distinct DNA and/or RNA sequences such as aptamers in a sequence-specific manner). Aptamers include nucleic acids that, through their ability to adopt a specific three-dimensional conformation, can bind to a target molecule with high affinity and specificity. Such adaptors can bind, for example, to a specific RNA sequence and secondary structure. These sequences (i.e., adaptor-binding elements) can be engineered into a guide RNA. For example, an MS2 aptamer can be engineered into a guide RNA to specifically bind an MS2 coat protein (MCP). For example, the adaptor can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the MCP sequence set forth in SEQ ID NO: 103.

Some specific examples of adaptors and targets include RNA-binding protein/aptamer combinations that exist within the diversity of bacteriophage coat proteins. For example, the following adaptor proteins or functional fragments or variants thereof can be used: MS2 coat protein (MCP), PP7, Qβ, F2, GA, fr, JP501, M12, R17, BZ13, JP34, JP500, KU1, M11, MX1, TW18, VK, SP, FI, ID2, NL95, TW19, AP205, φCb5, ΦCb8r, ΦCb12r, ΦCb23r, 7s, and PRR1. See, e.g., WO 2016/049258, herein incorporated by reference in its entirety for all purposes. A functional fragment or functional variant of an adaptor protein is one that retains the ability to bind to a specific adaptor-binding element (e.g., ability to bind to a specific adaptor-binding sequence in a sequence-specific manner). For example, a PP7 Pseudomonas bacteriophage coat protein variant can be used in which amino acids 68-69 are mutated to SG and amino acids 70-75 are deleted from the wild type protein. See, e.g., Wu et al. (2012) Biophys. J. 102(12):2936-2944 and Chao et al. (2007) Nat. Struct. Mol. Biol. 15(1):103-105, each of which is herein incorporated by reference in its entirety for all purposes. Likewise, an MCP variant may be used, such as a N55K mutant. See, e.g., Spingola and Peabody (1994) J. Biol. Chem. 269(12):9006-9010, herein incorporated by reference in its entirety for all purposes.

Other examples of adaptor proteins that can be used include all or part of (e.g., the DNA-binding from) endoribonuclease Csy4 or the lambda N protein. See, e.g., U S 2016/0312198, herein incorporated by reference in its entirety for all purposes.

(2) Transcriptional Activation Domains

The chimeric adaptor proteins disclosed herein comprise one or more transcriptional activation domains. Such transcriptional activation domains can be naturally occurring transcriptional activation domains, can be functional fragments or functional variants of naturally occurring transcriptional activation domains, or can be engineered or synthetic transcriptional activation domains. Transcriptional activation domains that can be used include those described for use in chimeric Cas proteins elsewhere herein.

A specific transcriptional activation domain for use in the chimeric adaptor proteins disclosed herein comprises p65 and/or HSF1 transcriptional activation domains or functional fragments or variants thereof. The HSF1 transcriptional activation domain can be a transcriptional activation domain of human heat shock factor 1 (HSF1). The p65 transcriptional activation domain can be a transcriptional activation domain of transcription factor p65, also known as nuclear factor NF-kappa-B p65 subunit encoded by the RELA gene. As one example, a transcriptional activation domain can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the p65 transcriptional activation domain protein sequence set forth in SEQ ID NO: 104. As another example, a transcriptional activation domain can comprise, consist essentially of, or consist of an amino acid sequence at least 85%, at least 90%, at least 91%, at least 92%, at least 93%, at least 94%, at least 95%, at least 96%, at least 97%, at least 98%, at least 99%, or 100% identical to the HSF1 transcriptional activation domain protein sequence set forth in SEQ ID NO: 105.

C. SAM Guide RNAs and Guide RNA Arrays

Also provided are guide RNAs or guide RNA arrays that can bind to the chimeric Cas proteins and chimeric adaptor proteins disclosed elsewhere herein to activate transcription of target genes. Nucleic acids encoding the guide RNAs can be genomically integrated in a cell or non-human animal (e.g., a SAM-ready cell or non-human animal) as disclosed elsewhere herein, or the guide RNAs or nucleic acids can be introduced into such cells and non-human animals using methods disclosed elsewhere herein (e.g., LNP-mediated delivery or AAV-mediated delivery). The delivery method can be selected to provide tissue-specific delivery of the recombinase as disclosed elsewhere herein.

A nucleic acid encoding the guide RNAs or guide RNA array can encode one or more guide RNAs (or if guide RNAs are being introduced into the cell or non-human animal, one or more guide RNAs can be introduced). For example, 2 or more, 3 or more, 4 or more, or 5 or more guide RNAs can be encoded or introduced. Each guide RNA coding sequence can be operably linked to the same promoter (e.g., a U6 promoter) or a different promoter (e.g., each guide RNA coding sequence is operably linked to its own U6 promoter). Two or more of the guide RNAs can target a different target sequence in a single target gene. For example, 2 or more, 3 or more, 4 or more, or 5 or more guide RNAs can each target a different target sequence in a single target gene. Similarly, the guide RNAs can target multiple target genes (e.g., 2 or more, 3 or more, 4 or more, or 5 or more target genes). Examples of guide RNA target sequences are disclosed elsewhere herein.

(1) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail, located downstream (3′) of the DNA-targeting segment, comprises, consists essentially of, or consists of GUUUUAGAGCUAUGCU (SEQ ID NO: 142). Any of the DNA-targeting segments disclosed herein can be joined to the 5′ end of SEQ ID NO: 142 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences comprise, consist essentially of, or consist of any one of

(SEQ ID NO: 143)
AGCAUAGCAAGUUAAAAUAAGGCUAGUCCGUUAUCAACUU
GAAAAAGUGGCACCGAGUCGGUGCUUU,
(SEQ ID NO: 144)
AAACAGCAUAGCAAGUUAAAAUAAGGCUAGUCCGUUAUCA
ACUUGAAAAAGUGGCACCGAGUCGGUGCUUUU,
or
(SEQ ID NO: 145)
GUUGGAACCAUUCAAAACAGCAUAGCAAGUUAAAAUAAGG
CUAGUCCGUUAUCAACUUGAAAAAGUGGCACCGAGUCGGU
GC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, about 18, about 19, or about 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, about or more than about 26, about or more than about 32, about or more than about 45, about or more than about 48, about or more than about 54, about or more than about 63, about or more than about 67, about or more than about 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences comprise, consist essentially of, or consist of: GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 146); GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 147); GUUUUAGAGCUAGAAAUAGCAAGUUAAAAUAAGGCUAGUCCGUUAUCAACUUGA AAAAGUGGCACCGAGUCGGUGC (version 3; SEQ ID NO: 148); GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 149); GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 150); GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 151); or GUUUAAGAGCUAUGCUGGAAACAGCAUAGCAAGUUUAAAUAAGGCUAGUCCGUU AUCAACUUGAAAAAGUGGCACCGAGUCGGUGC (version 7; SEQ ID NO: 152). Guide RNAs targeting any of the guide RNA target sequences disclosed herein (e.g., any of SEQ ID NOS: 121-123) can include, for example, a DNA-targeting segment (e.g., any of SEQ ID NOS: 128-130) on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). Guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, such as transcriptional activators); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Unmodified nucleic acids can be prone to degradation. Exogenous nucleic acids can also induce an innate immune response. Modifications can help introduce stability and reduce immunogenicity. Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage; (2) alteration or replacement of a constituent of the ribose sugar such as alteration or replacement of the 2′ hydroxyl on the ribose sugar; (3) replacement of the phosphate moiety with dephospho linkers; (4) modification or replacement of a naturally occurring nucleobase; (5) replacement or modification of the ribose-phosphate backbone; (6) modification of the 3′ end or 5′ end of the oligonucleotide (e.g., removal, modification or replacement of a terminal phosphate group or conjugation of a moiety); and (7) modification of the sugar. Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

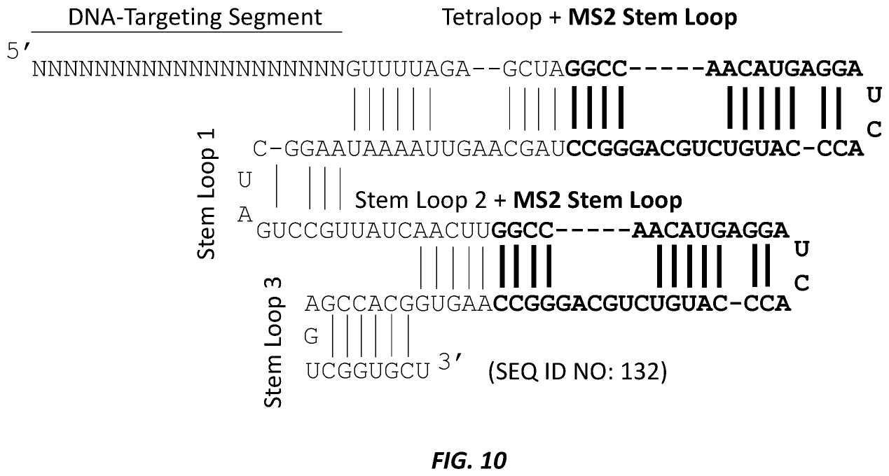

In some guide RNAs (e.g., single guide RNAs), at least one loop (e.g., two loops) of the guide RNA is modified by insertion of a distinct RNA sequence that binds to one or more adaptors (i.e., adaptor proteins or domains). Such adaptor proteins can be used to further recruit one or more heterologous functional domains, such as transcriptional activation domains. Examples of fusion proteins comprising such adaptor proteins (i.e., chimeric adaptor proteins) are disclosed elsewhere herein. For example, an MS2-binding loop ggccAACAUGAGGAUCACCCAUGUCUGCAGggcc (SEQ ID NO: 106) may replace nucleotides +13 to +16 and nucleotides +53 to +56 of the sgRNA scaffold (backbone) set forth in SEQ ID NO: 146, 148, 150, or 151 or the sgRNA backbone for the S. pyogenes CRISPR/Cas9 system described in WO 2016/049258 and Konermann et al. (2015) Nature 517(7536):583-588, each of which is herein incorporated by reference in its entirety for all purposes. See, e.g., FIG. 10. The guide RNA numbering used herein refers to the nucleotide numbering in the guide RNA scaffold sequence (i.e., the sequence downstream of the DNA-targeting segment of the guide RNA). For example, the first nucleotide of the guide RNA scaffold is +1, the second nucleotide of the scaffold is +2, and so forth. Residues corresponding with nucleotides +13 to +16 in SEQ ID NO: 146, 148, 150, or 151 are the loop sequence in the region spanning nucleotides +9 to +21 in SEQ ID NO: 146, 148, 150, or 151, a region referred to herein as the tetraloop. Residues corresponding with nucleotides +53 to +56 in SEQ ID NO: 146, 148, 150, or 151 are the loop sequence in the region spanning nucleotides +48 to +61 in SEQ ID NO: 146, 148, 150, or 151, a region referred to herein as the stem loop 2. Other stem loop sequences in SEQ ID NO: 146, 148, 150, or 151 comprise stem loop 1 (nucleotides +33 to +41) and stem loop 3 (nucleotides +63 to +75). The resulting structure is an sgRNA scaffold in which each of the tetraloop and stem loop 2 sequences have been replaced by an MS2 binding loop. The tetraloop and stem loop 2 protrude from the Cas9 protein in such a way that adding an MS2-binding loop should not interfere with any Cas9 residues. Additionally, the proximity of the tetraloop and stem loop 2 sites to the DNA indicates that localization to these locations could result in a high degree of interaction between the DNA and any recruited protein, such as a transcriptional activator. Thus, in some sgRNAs, nucleotides corresponding to +13 to +16 and/or nucleotides corresponding to +53 to +56 of the guide RNA scaffold set forth in SEQ ID NO: 146, 148, 150, or 151 or corresponding residues when optimally aligned with any of these scaffold/backbones are replaced by the distinct RNA sequences capable of binding to one or more adaptor proteins or domains. Alternatively or additionally, adaptor-binding sequences can be added to the 5′ end or the 3′ end of a guide RNA. An exemplary guide RNA scaffold comprising MS2-binding loops in the tetraloop and stem loop 2 regions can comprise, consist essentially of, or consist of the sequence set forth in SEQ ID NO: 127 or 140. An exemplary generic single guide RNA comprising MS2-binding loops in the tetraloop and stem loop 2 regions can comprise, consist essentially of, or consist of the sequence set forth in SEQ ID NO: 132 or 141.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

(2) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

It can be preferable for the target sequence to be adjacent to the transcription start site of a gene. For example, the target sequence can be within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair of the transcription start site, within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair upstream of the transcription start site, or within 1000, 900, 800, 700, 600, 500, 400, 300, 200, 190, 180, 170, 160, 150, 140, 130, 120, 110, 100, 90, 80, 70, 60, 50, 40, 30, 20, 10, 5, or 1 base pair downstream of the transcription start site. Optionally, the target sequence is within the region 200 base pairs upstream of the transcription start site and 1 base pair downstream of the transcription start site (−200 to +1).

The target sequence can be within any gene desired to be targeted for transcriptional activation. In some cases, a target gene may be one that is a non-expressing gene or a weakly expressing gene (e.g., only minimally expressed above background, such as 1.1-fold, 1.2-fold, 1.3-fold, 1.4-fold, 1.5-fold, 1.6-fold, 1.7-fold, 1.8-fold, 1.9-fold, or 2-fold). The target gene may also be one that is expressed at low levels compared to a control gene. The target gene may also be one that is epigenetically silenced. The term “epigenetically silenced” refers to a gene that is not being transcribed or is being transcribed at a level that is decreased with respect to the level of transcription of the gene in a control sample (e.g., a corresponding control cell, such as a normal cell), due to a mechanism other than a genetic change such as a mutation. Epigenetic mechanisms of gene silencing are well known and include, for example, hypermethylation of CpG dinucleotides in a CpG island of the 5′ regulatory region of a gene and structural changes in chromatin due, for example, to histone acetylation, such that gene transcription is reduced or inhibited.

Target genes can include genes expressed in particular organs or tissues, such as the liver. Target genes can include disease-associated genes. A disease-associated gene refers to any gene that yields transcription or translation products at an abnormal level or in an abnormal form in cells derived from a disease-affected tissues compared with tissues or cells of a non-disease control. It may be a gene that becomes expressed at an abnormally high level, where the altered expression correlates with the occurrence and/or progression of the disease. A disease-associated gene also refers to a gene possessing a mutation or genetic variation that is responsible for the etiology of a disease. The transcribed or translated products may be known or unknown, and may be at a normal or abnormal level. For example, target genes can be genes associated with protein aggregation diseases and disorders, such as Alzheimer's disease, Parkinson's disease, Huntington's disease, amyotrophic lateral sclerosis, prion diseases, and amyloidoses such as transthyretin amyloidosis (e.g., Ttr). Target genes can also be genes involved in pathways related to a disease or condition, such as hypercholesterolemia or atherosclerosis, or genes that when overexpressed can model such diseases or conditions. Target genes can also be genes expressed or overexpressed in one or more types of cancer. See, e.g., Santarius et al. (2010) Nat. Rev. Cancer 10(1):59-64, herein incorporated by reference in its entirety for all purposes.

One specific example of such a target gene is the Ttr gene (e.g., the humanized TTR locus described elsewhere herein). Examples of guide RNA target sequences (not including PAM) in the mouse Ttr gene are set forth in SEQ ID NOS: 121, 122, and 123, respectively. SEQ ID NO: 121 is located −63 of the Ttr transcription start site (genomic coordinates: build mm10, chr18, +strand, 20665187-20665209), SEQ ID NO: 122 is located −134 of the Ttr transcription start site (genomic coordinates: build mm10, chr18, +strand, 20665116-20665138), and SEQ ID NO: 123 is located −112 of the Ttr transcription start site (genomic coordinates: build mm10, chr18, +strand, 20665138-20665160). Guide RNA DNA-targeting segments corresponding to the guide RNA target sequences set forth in SEQ ID NOS: 121, 122, and 123, respectively, are set forth in SEQ ID NOS: 128, 129, and 130, respectively. Examples of single guide RNAs comprising these DNA-targeting segments are set forth in SEQ ID NOS: 124, 125, and 126, respectively.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 153) or N₂₀NGG (SEQ ID NO: 154). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 155) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 153-155, including the 5′ G or GG and the 3′ GG or NGG. Yet other guide RNA target sequences plus PAMs can have between 14 and 20 nucleotides in length of SEQ ID NOS: 153-155.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, at least 3, at least 4, at least 5, at least 6, at least 7, at least 8, at least 9, at least 10, at least 15, at least 20, at least 25, at least 30, at least 40, at least 50, at least 75, at least 100, at least 250, at least 500, or at least 1,000 base pairs.

D. Recombinases and Recombinase Deleter Non-Human Animals

Cells or non-human animals comprising a chimeric Cas protein expression cassette, a chimeric adaptor protein expression cassette, a SAM expression cassette, a guide RNA expression cassette, or a recombinase expression cassette in which the cassette is downstream of a polyadenylation signal or transcription terminator flanked by recombinase recognition sites recognized by a site-specific recombinase as disclosed herein can further comprise a recombinase expression cassette that drives expression of the site-specific recombinase. A nucleic acid encoding the recombinase can be genomically integrated, or the recombinase or nucleic acids can be introduced into such cells and non-human animals using methods disclosed elsewhere herein (e.g., LNP-mediated delivery or AAV-mediated delivery). The delivery method can be selected to provide tissue-specific delivery of the recombinase as disclosed elsewhere herein.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The recombinase expression cassette can be integrated at a different target genomic locus from other expression cassettes disclosed herein, or it can be genomically integrated at the same target locus (e.g., a Rosa26 locus, such as integrated in the first intron of the Rosa26 locus). For example, the cell or non-human animal can be heterozygous for each of a SAM expression cassette (or chimeric Cas protein expression cassette or chimeric adaptor protein expression cassette) and the recombinase expression cassette, with one allele of the target genomic locus comprising the SAM expression cassette, and a second allele of the target genomic locus comprising the recombinase expression cassette expression cassette. Likewise, the cell or non-human animal can be heterozygous for each of a guide RNA expression cassette (e.g., guide RNA array expression cassette) and the recombinase expression cassette, with one allele of the target genomic locus comprising the guide RNA expression cassette, and a second allele of the target genomic locus comprising the recombinase expression cassette expression cassette.

The recombinase gene in a recombinase expression cassette can be operably linked to any suitable promoter. Examples of promoters are disclosed elsewhere herein. For example, the promoter can be a tissue-specific promoter or a developmental-stage-specific promoter. Such promoters are advantageous because they can selectively activate transcription of a target gene in a desired tissue or only at a desired developmental stage. For example, in the case of Cas proteins, this can reduce the possibility of Cas-mediated toxicity in vivo. Exemplary promoters for mouse recombinase delete strains are known and are provided, for example, in US 2019/0284572 and WO 2019/183123, each of which is herein incorporated by reference in its entirety for all purposes. As a specific example, an albumin (Alb) promoter can be used for liver-specific expression.

E. Nucleic Acids Encoding Chimeric Cas Protein, Chimeric Adaptor Protein, Guide RNA, Synergistic Activation Mediator, or Recombinase

Also provided are nucleic acids encoding a chimeric Cas protein, a chimeric adaptor protein, a guide RNA, a recombinase, or any combination thereof. Chimeric Cas proteins, chimeric adaptor proteins, guide RNAs, and recombinases are described in more detail elsewhere herein. For example, the nucleic acids can be chimeric Cas protein expression cassettes, chimeric adaptor protein expression cassettes, synergistic activation mediator (SAM) expression cassettes comprising nucleic acids encoding both a chimeric Cas protein and a chimeric adaptor protein, guide RNA or guide RNA array expression cassettes, recombinase expression cassettes, or any combination thereof. Such nucleic acids can be RNA (e.g., messenger RNA (mRNA)) or DNA, can be single-stranded or double-stranded, and can be linear or circular. DNA can be part of a vector, such as an expression vector or a targeting vector. The vector can also be a viral vector such as adenoviral, adeno-associated viral, lentiviral, and retroviral vectors. When any of the nucleic acids disclosed herein is introduced into a cell, the encoded chimeric Cas protein, chimeric adaptor protein, or guide RNA can be transiently, conditionally, or constitutively expressed in the cell.

Optionally, the nucleic acids can be codon-optimized for efficient translation into protein in a particular cell or organism. For example, the nucleic acid can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence.

The nucleic acids or expression cassettes can be stably integrated into the genome (i.e., into a chromosome) of the cell or non-human animal or it can be located outside of a chromosome (e.g., extrachromosomally replicating DNA). The stably integrated expression cassettes or nucleic acids can be randomly integrated into the genome of the non-human animal (i.e., transgenic), or they can be integrated into a predetermined region of the genome of the non-human animal (i.e., knock in). In one example, a nucleic acid or expression cassette is stably integrated into a safe harbor locus as described elsewhere herein. The target genomic locus at which a nucleic acid or expression cassette is stably integrated can be heterozygous for the nucleic acid or expression cassette or homozygous for the nucleic acid or expression cassette. For example, a target genomic locus or a cell or non-human animal can be heterozygous for a SAM expression cassette and heterozygous for a guide RNA expression cassette, optionally with each being at the same target genomic locus on different alleles.

A nucleic acid or expression cassette described herein can be operably linked to any suitable promoter for expression in vivo within a non-human animal or in vitro or ex vivo within a cell. The non-human animal can be any suitable non-human animal as described elsewhere herein. As one example, a nucleic acid or expression cassette (e.g., a chimeric Cas protein expression cassette, a chimeric adaptor protein expression cassette, or a SAM cassette comprising nucleic acids encoding both a chimeric Cas protein and a chimeric adaptor protein) can be operably linked to an endogenous promoter at a target genomic locus, such as a Rosa26 promoter. Alternatively, cassette nucleic acid or expression cassette can be operably linked to an exogenous promoter, such as a constitutively active promoter (e.g., a CAG promoter or a U6 promoter), a conditional promoter, an inducible promoter, a temporally restricted promoter (e.g., a developmentally regulated promoter), or a spatially restricted promoter (e.g., a cell-specific or tissue-specific promoter). Such promoters are well-known and are discussed elsewhere herein. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, or a zygote. Such promoters can be, for example, conditional promoters, inducible promoters, constitutive promoters, or tissue-specific promoters.

For example, a nucleic acid encoding a guide RNA can be operably linked to a U6 promoter, such as a human U6 promoter or a mouse U6 promoter. Specific examples of suitable promoters (e.g., for expressing a guide RNA) include an RNA polymerase III promoter, such as a human U6 promoter, a rat U6 polymerase III promoter, or a mouse U6 polymerase III promoter.

Optionally, the promoter can be a bidirectional promoter driving expression of one gene (e.g., a gene encoding a chimeric Cas protein) and a second gene (e.g., a gene encoding a guide RNA or a chimeric adaptor protein) in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express two genes simultaneously allows for the generation of compact expression cassettes to facilitate delivery.

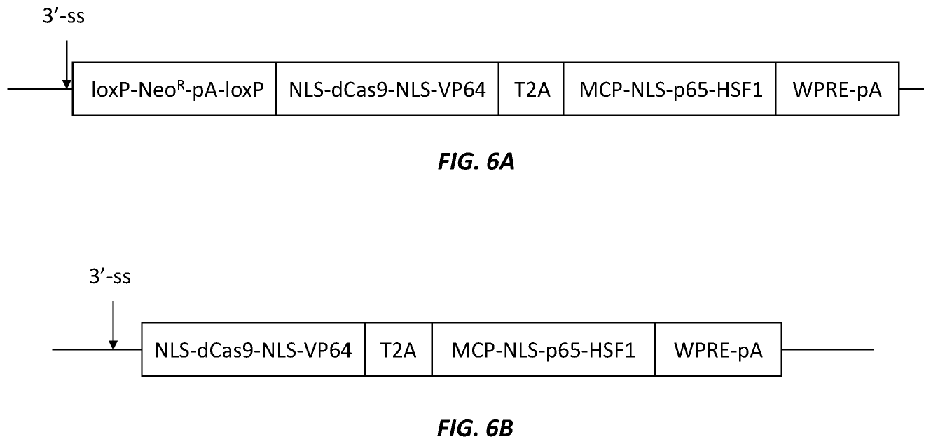

One or more of the nucleic acids can be together in a multicistronic expression construct. For example, a nucleic acid encoding a chimeric Cas protein and a nucleic acid encoding a chimeric adaptor protein can be together in a bicistronic expression construct. See, e.g., FIGS. 6A and 6B. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). For example, such constructs can comprise: (1) nucleic acids encoding one or more chimeric Cas proteins and one or more chimeric adaptor proteins; (2) nucleic acids encoding two or more chimeric adaptor proteins; (3) nucleic acids encoding two or more chimeric Cas proteins; (4) nucleic acids encoding two or more guide RNAs or two or more guide RNA arrays; (5) nucleic acids encoding one or more chimeric Cas proteins and one or more guide RNAs or guide RNA arrays; (6) nucleic acids encoding one or more chimeric adaptor proteins and one or more guide RNAs or guide RNA arrays; or (7) nucleic acids encoding one or more chimeric Cas proteins, one or more chimeric adaptor proteins, and one or more guide RNAs or guide RNA arrays. As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4):e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin. Biol. Ther. 5(5):627-638, herein incorporated by reference in its entirety for all purposes. Examples of 2A peptides that can be used include Thoseaasigna virus 2A (T2A); porcine teschovirus-1 2A (P2A); equine rhinitis A virus (ERAV) 2A (E2A); and FMDV 2A (F2A). Exemplary T2A, P2A, E2A, and F2A sequences include the following: T2A (EGRGSLLTCGDVEENPGP; SEQ ID NO: 107); P2A (ATNFSLLKQAGDVEENPGP; SEQ ID NO: 108); E2A (QCTNYALLKLAGDVESNPGP; SEQ ID NO: 109); and F2A (VKQTLNFDLLKLAGDVESNPGP; SEQ ID NO: 110). GSG residues can be added to the 5′ end of any of these peptides to improve cleavage efficiency.

Any of the nucleic acids or expression cassettes can also comprise a polyadenylation signal or transcription terminator upstream of a coding sequence. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, a chimeric Cas protein expression cassette, a chimeric adaptor protein expression cassette, a SAM expression cassette, a guide RNA expression cassette, or a recombinase expression cassette can comprise a polyadenylation signal or transcription terminator upstream of the coding sequence(s) in the expression cassette. The polyadenylation signal or transcription terminator can be flanked by recombinase recognition sites recognized by a site-specific recombinase. Optionally, the recombinase recognition sites also flank a selection cassette comprising, for example, the coding sequence for a drug resistance protein. Optionally the recombinase recognition sites do not flank a selection cassette. The polyadenylation signal or transcription terminator prevents transcription and expression of the protein or RNA encoded by the coding sequence (e.g., chimeric Cas protein, chimeric adaptor protein, guide RNA, or recombinase). However, upon exposure to the site-specific recombinase, the polyadenylation signal or transcription terminator will be excised, and the protein or RNA can be expressed.

Such a configuration for an expression cassette (e.g., a chimeric Cas protein expression cassette or a SAM expression cassette) can enable tissue-specific expression or developmental-stage-specific expression in non-human animals comprising the expression cassette if the polyadenylation signal or transcription terminator is excised in a tissue-specific or developmental-stage-specific manner. For example, in the case of the chimeric Cas protein, this may reduce toxicity due to prolonged expression of the chimeric Cas protein in a cell or non-human animal or expression of the chimeric Cas protein at undesired developmental stages or in undesired cell or tissue types within a non-human animal. See, e.g., Parikh et al. (2015) PLoS One 10(1):e0116484, herein incorporated by reference in its entirety for all purposes. Excision of the polyadenylation signal or transcription terminator in a tissue-specific or developmental-stage-specific manner can be achieved if a non-human animal comprising the expression cassette further comprises a coding sequence for the site-specific recombinase operably linked to a tissue-specific or developmental-stage-specific promoter. The polyadenylation signal or transcription terminator will then be excised only in those tissues or at those developmental stages, enabling tissue-specific expression or developmental-stage-specific expression. In one example, a chimeric Cas protein, a chimeric adaptor protein, a chimeric Cas protein and a chimeric adaptor protein, or a guide RNA can be expressed in a liver-specific manner. Examples of such promoters that have been used to develop such “recombinase deleter” strains of non-human animals are disclosed elsewhere herein.

Any transcription terminator or polyadenylation signal can be used. A “transcription terminator” as used herein refers to a DNA sequence that causes termination of transcription. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells.

Site-specific recombinases include enzymes that can facilitate recombination between recombinase recognition sites, where the two recombination sites are physically separated within a single nucleic acid or on separate nucleic acids. Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

The expression cassettes disclosed herein can comprise other components as well. Such expression cassettes (e.g., chimeric Cas protein expression cassette, chimeric adaptor protein expression cassette, SAM expression cassette, guide RNA expression cassette, or recombinase expression cassette) can further comprise a 3′ splicing sequence at the 5′ end of the expression cassette and/or a second polyadenylation signal following the coding sequence (e.g., encoding the chimeric Cas protein, the chimeric adaptor protein, the guide RNA, or the recombinase). The term 3′ splicing sequence refers to a nucleic acid sequence at a 3′ intron/exon boundary that can be recognized and bound by splicing machinery. An expression cassette can further comprise a selection cassette comprising, for example, the coding sequence for a drug resistance protein. Examples of suitable selection markers include neomycin phosphotransferase (neo^{r}), hygromycin B phosphotransferase (hyg^{r}), puromycin-N-acetyltransferase (puro^{r}), blasticidin S deaminase (bsr^{r}), xanthine/guanine phosphoribosyl transferase (gpt), and herpes simplex virus thymidine kinase (HSV-k). Optionally, the selection cassette can be flanked by recombinase recognition sites for a site-specific recombinase. If the expression cassette also comprises recombinase recognition sites flanking a polyadenylation signal upstream of the coding sequence as described above, the selection cassette can be flanked by the same recombinase recognition sites or can be flanked by a different set of recombinase recognition sites recognized by a different recombinase.

An expression cassette can also comprise a nucleic acid encoding one or more reporter proteins, such as a fluorescent protein (e.g., a green fluorescent protein). Any suitable reporter protein can be used. For example, a fluorescent reporter protein as defined elsewhere herein can be used, or a non-fluorescent reporter protein can be used. Examples of fluorescent reporter proteins are provided elsewhere herein. Non-fluorescent reporter proteins include, for example, reporter proteins that can be used in histochemical or bioluminescent assays, such as beta-galactosidase, luciferase (e.g., Renilla luciferase, firefly luciferase, and NanoLuc luciferase), and beta-glucuronidase. An expression cassette can include a reporter protein that can be detected in a flow cytometry assay (e.g., a fluorescent reporter protein such as a green fluorescent protein) and/or a reporter protein that can be detected in a histochemical assay (e.g., beta-galactosidase protein). One example of such a histochemical assay is visualization of in situ beta-galactosidase expression histochemically through hydrolysis of X-Gal (5-bromo-4-chloro-3-indoyl-b-D-galactopyranoside), which yields a blue precipitate, or using fluorogenic substrates such as beta-methyl umbelliferyl galactoside (MUG) and fluorescein digalactoside (FDG).

The expression cassettes described herein can be in any form. For example, an expression cassette can be in a vector or plasmid, such as a viral vector. The expression cassette can be operably linked to a promoter in an expression construct capable of directing expression of a protein or RNA (e.g., upon removal of an upstream polyadenylation signal). Alternatively, an expression cassette can be in a targeting vector. For example, the targeting vector can comprise homology arms flanking the expression cassette, wherein the homology arms are suitable for directing recombination with a desired target genomic locus to facilitate genomic integration and/or replacement of endogenous sequence.

The expression cassettes described herein can be in vitro, they can be within a cell (e.g., an embryonic stem cell) ex vivo (e.g., genomically integrated or extrachromosomal), or they can be in an organism (e.g., a non-human animal) in vivo (e.g., genomically integrated or extrachromosomal). If ex vivo, the expression cassette(s) can be in any type of cell from any organism, such as a totipotent cell such as an embryonic stem cell (e.g., a mouse or a rat embryonic stem cell) or an induced pluripotent stem cell (e.g., a human induced pluripotent stem cell). If in vivo, the expression cassette(s) can be in any type of organism (e.g., a non-human animal as described further elsewhere herein).

A specific example of a nucleic acid encoding a catalytically inactive Cas protein can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the dCas9 protein sequence set forth in SEQ ID NO: 98. Optionally, the nucleic acid can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 111 (optionally wherein the sequence encodes a protein at least 85%, at least 90%, at least 91%, at least 92%, at least 93%, at least 94%, at least 95%, at least 96%, at least 97%, at least 98%, at least 99%, or 100% identical to the dCas9 protein sequence set forth in SEQ ID NO: 98).

A specific example of a nucleic acid encoding a chimeric Cas protein can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the chimeric Cas protein sequence set forth in SEQ ID NO: 97. Optionally, the nucleic acid can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 112 (optionally wherein the sequence encodes a protein at least 85%, at least 90%, at least 91%, at least 92%, at least 93%, at least 94%, at least 95%, at least 96%, at least 97%, at least 98%, at least 99%, or 100% identical to the chimeric Cas protein sequence set forth in SEQ ID NO: 97).

A specific example of a nucleic acid encoding an adaptor can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to MCP sequence set forth in SEQ ID NO: 103. Optionally, the nucleic acid can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 113 (optionally wherein the sequence encodes a protein at least 85%, at least 90%, at least 91%, at least 92%, at least 93%, at least 94%, at least 95%, at least 96%, at least 97%, at least 98%, at least 99%, or 100% identical to the MCP sequence set forth in SEQ ID NO: 103).

A specific example of a nucleic acid encoding a chimeric adaptor protein can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the chimeric adaptor protein sequence set forth in SEQ ID NO: 102. Optionally, the nucleic acid can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 114 (optionally wherein the sequence encodes a protein at least 85%, at least 90%, at least 91%, at least 92%, at least 93%, at least 94%, at least 95%, at least 96%, at least 97%, at least 98%, at least 99%, or 100% identical to the chimeric adaptor protein sequence set forth in SEQ ID NO: 102).

Specific examples of nucleic acids encoding transcriptional activation domains can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the VP64, p65, or HSF1 sequences set forth in SEQ ID NO: 99, 104, or 105, respectively. Optionally, the nucleic acid can comprise, consist essentially of, or consist of a nucleic acid encoding an amino acid sequence at least 85%, at least 90%, at least 91%, at least 92%, at least 93%, at least 94%, at least 95%, at least 96%, at least 97%, at least 98%, at least 99%, or 100% identical to the sequence set forth in SEQ ID NO: 115, 116, or 117, respectively (optionally wherein the sequence encodes a protein at least 85%, at least 90%, at least 91%, at least 92%, at least 93%, at least 94%, at least 95%, at least 96%, at least 97%, at least 98%, at least 99%, or 100% identical to the VP64, p65, or HSF1 sequences set forth in SEQ ID NO: 99, 104, or 105, respectively).

One exemplary synergistic activation mediator (SAM) expression cassette comprises from 5′ to 3′: (a) a 3′ splicing sequence; (b) a first recombinase recognition site (e.g., loxP site); (c) a coding sequence for a drug resistance gene (e.g., neomycin phosphotransferase (neo^{r}) coding sequence); (d) a polyadenylation signal; (e) a second recombinase recognition site (e.g., loxP site); (f) a chimeric Cas protein coding sequence (e.g., dCas9-NLS-VP64 fusion protein or NLS-dCas9-NLS-VP64 fusion protein); (g) a 2A protein coding sequence (e.g., a P2A or T2A coding sequence); and (e) a chimeric adaptor protein coding sequence (e.g., MCP-NLS-p65-HSF1). Another exemplary synergistic activation mediator (SAM) expression cassette comprises from 5′ to 3′: (a) a 3′ splicing sequence; (b) a first recombinase recognition site (e.g., loxP site); (c) a coding sequence for a drug resistance gene (e.g., neomycin phosphotransferase (neo^{r}) coding sequence); (d) a polyadenylation signal (e.g., PGK polyadenylation signal and/or SV40 polyadenylation signal, such as a combination of a PGK polyadenylation signal and 3 SV40 polyadenylation signals); (e) a second recombinase recognition site (e.g., loxP site); (f) a chimeric Cas protein coding sequence (e.g., dCas9-NLS-VP64 fusion protein or NLS-dCas9-NLS-VP64 fusion protein); (g) a 2A protein coding sequence (e.g., a P2A or T2A coding sequence);(e) a chimeric adaptor protein coding sequence (e.g., MCP-NLS-p65-HSF1); (f) a Woodchuck hepatitis virus posttranscriptional regulatory element (WPRE); and (g) another polyadenylation signal (e.g., BGH polyadenylation signal). See, e.g., FIG. 6A and SEQ ID NO: 118 (coding sequence set forth in SEQ ID NO: 133 and encoding protein set forth in SEQ ID NO: 131).

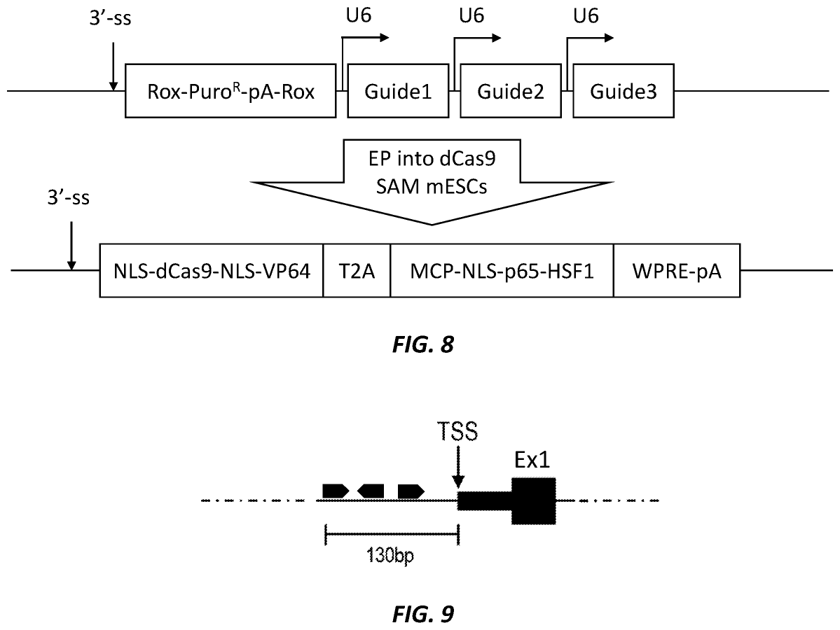

One exemplary generic guide RNA array expression cassette comprises from 5′ to 3′: (a) a 3′ splicing sequence; (b) a first recombinase recognition site (e.g., rox site); (c) a coding sequence for a drug resistance gene (e.g., puromycin-N-acetyltransferase (puro^{r}) coding sequence); (d) a polyadenylation signal (e.g., PGK polyadenylation signal and/or SV40 polyadenylation signal, such as a combination of a PGK polyadenylation signal and 3 SV40 polyadenylation signals); (e) a second recombinase recognition site (e.g., rox site); (f) a guide RNA array comprising one or more guide RNA genes (e.g., a first U6 promoter followed by a first guide RNA coding sequence and a first terminator sequence, a second U6 promoter followed by a second guide RNA coding sequence and a second terminator sequence, and a third U6 promoter followed by a third guide RNA coding sequence and a third terminator sequence). See, e.g., FIG. 8 and SEQ ID NO: 119. The region of SEQ ID NO: 119 comprising the promoters and guide RNA coding sequences is set forth in SEQ ID NO: 134. The recombinase recognition sites in the guide RNA array expression cassette can be the same or different from the recombinase recognition sites in the SAM expression cassette (e.g., can be recognized by the same recombinase or a different recombinase). Such an exemplary guide RNA array expression cassette encoding guide RNAs targeting mouse Ttr is set forth in SEQ ID NO: 120. The region of SEQ ID NO: 120 comprising the promoters and guide RNA coding sequences is set forth in SEQ ID NO: 135.

Another exemplary generic guide RNA array expression cassette comprises one or more guide RNA genes (e.g., a first U6 promoter followed by a first guide RNA coding sequence, a second U6 promoter followed by a second guide RNA coding sequence, and a third U6 promoter followed by a third guide RNA coding sequence). Such an exemplary generic guide RNA array expression cassette is set forth in SEQ ID NO: 134. Examples of such guide RNA array expression cassettes for specific genes are set forth, e.g., in SEQ ID NOS: 120, 135, and 136.

F. Genomic Loci for Integration

The nucleic acids and expression cassettes described herein can be genomically integrated at a target genomic locus in a cell or a non-human animal. Any target genomic locus capable of expressing a gene can be used.

An example of a target genomic locus into which the nucleic acids or cassettes described herein can be stably integrated is a safe harbor locus in the genome of the non-human animal. Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

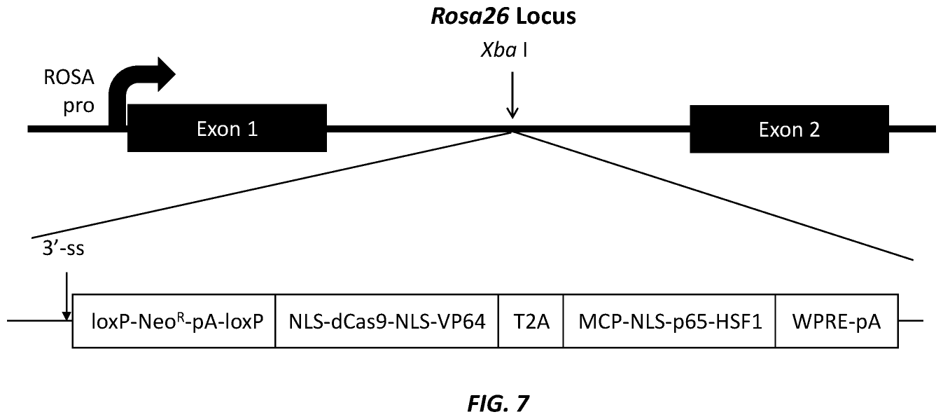

For example, the Rosa26 locus and its equivalent in humans offer an open chromatin configuration in all tissues and is ubiquitously expressed during embryonic development and in adults. See, e.g., Zambrowicz et al. (1997) Proc. Natl. Acad. Sci. USA 94:3789-3794, herein incorporated by reference in its entirety for all purposes. In addition, the Rosa26 locus can be targeted with high efficiency, and disruption of the Rosa26 gene produces no overt phenotype. Other examples of safe harbor loci include CCR5, HPRT, AAVS1, and albumin. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Biallelic targeting of safe harbor loci such as the Rosa26 locus has no negative consequences, so different genes or reporters can be targeted to the two Rosa26 alleles. In one example, an expression cassette is integrated into an intron of the Rosa26 locus, such as the first intron of the Rosa26 locus. See, e.g., FIG. 7.

Expression cassettes integrated into a target genomic locus can be operably linked to an endogenous promoter at the target genomic locus or can be operably linked to an exogenous promoter that is heterologous to the target genomic locus. In one example, a chimeric Cas protein expression cassette, chimeric adaptor protein expression cassette, or synergistic activation mediator (SAM) expression cassette is integrated into a target genomic locus (e.g., the Rosa26 locus) and is operably linked to the endogenous promoter at the target genomic locus (e.g., the Rosa26 promoter). In another example, a guide RNA expression cassette is integrated into a target genomic locus (e.g., the Rosa26 locus) and is operably linked to one or more heterologous promoters (e.g., U6 promoter(s), such as a different U6 promoter upstream of each guide RNA coding sequence).

### IV. Methods of Using Non-Human Animals Comprising a Humanized TTR Locus Comprising a V30M Mutation for Assessing Efficacy of Human-TTR-Targeting Reagents In Vivo or Ex Vivo

Various methods are provided for using the non-human animals comprising a humanized TTR locus comprising a V30M mutation as described elsewhere herein for assessing or optimizing delivery or efficacy of human-TTR-targeting reagents (e.g., therapeutic molecules or complexes) in vivo or ex vivo or in vitro. Because the non-human animals comprise a humanized TTR locus, the non-human animals will more accurately reflect the efficacy of a human TTR-targeting reagent. Such non-human animals are particularly useful for testing genome-editing reagents designed to target the human TTR gene because the non-human animals disclosed herein comprise humanized endogenous TTR loci rather than transgenic insertions of human TTR sequence at random genomic loci, and the humanized endogenous TTR loci comprise orthologous human genomic TTR sequence from both coding and non-coding regions (e.g., from both exonic and intronic regions) rather than an artificial cDNA sequence.

A. Methods of Testing Efficacy of Human-TTR-Targeting Reagents In Vivo or Ex Vivo

Various methods are provided for assessing delivery or efficacy of human-TTR-targeting reagents in vivo using non-human animals comprising a humanized TTR locus comprising a V30M mutation as described elsewhere herein. Such methods can comprise: (a) introducing into the non-human animal a human-TTR-targeting reagent; and (b) assessing the activity of the human-TTR-targeting reagent. The assessing can be, for example, compared to a control non-human animal comprising the humanized TTR locus that was not administered the human-TTR-targeting reagent or compared to the non-human animal prior to administration of the human-TTR-targeting reagent.

In methods in which the non-human animals also comprise CRISPR/Cas synergistic activation mediator system components, such methods can further comprise administering one or more SAM guide RNAs or one or more DNAs encoding one or more SAM guide RNAs as described elsewhere herein to the non-human animal prior to step (a), wherein each of the one or more guide RNAs comprises one or more adaptor-binding elements to which the chimeric adaptor protein can specifically bind, and wherein each of the one or more guide RNAs forms a complex with the chimeric Cas protein and the chimeric adaptor protein and guides them to a target sequence within the humanized Ttr locus, thereby increasing expression of the humanized Ttr locus. If the assessing step is performed compared to a control non-human animal that was not administered the human-TTR-targeting reagent, the methods can further comprise administering the one or more guide RNAs or the one or more DNAs encoding one or more SAM guide RNAs as described elsewhere herein to the control non-human animal.

In methods further comprising administering one or more SAM guide RNAs or one or more DNAs encoding one or more SAM guide RNAs as described elsewhere herein to the non-human animal prior to step (a), any suitable amount of time can take place between the step of administering one or more SAM guide RNAs or one or more DNAs encoding one or more SAM guide RNAs and the step of administering the human-TTR-targeting reagent. In one example, the human-TTR-targeting reagent can be administered at least about 1 day, at least about 2 days, at least about 3 days, at least about 4 days, at least about 5 days, at least about 6 days, at least about 7 days, at least about 8 days, at least about 9 days, at least about 10 days, at least about 15 days, at least about 20 days, at least about 25 days, or at least about 30 days after administering the one or more guide RNAs or the one or more DNAs encoding the one or more guide RNAs. In another example, the human-TTR-targeting reagent is administered about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 15 days, about 1 day to about 20 days, about 1 day to about 25 days, or about 1 day to about 30 days after administering the one or more guide RNAs or the one or more DNAs encoding the one or more guide RNAs. In another example, the human-TTR-targeting reagent is administered about 1 day to about 30 days, about 2 days to about 30 days, about 3 days to about 30 days, about 4 days to about 30 days, about 5 days to about 30 days, about 6 days to about 30 days, about 7 days to about 30 days, about 8 days to about 30 days, about 9 days to about 30 days, about 10 days to about 30 days, about 15 days to about 30 days, about 20 days to about 30 days, or about 25 days to about 30 days after administering the one or more guide RNAs or the one or more DNAs encoding the one or more guide RNAs.

Such methods can further comprise measuring expression of a Ttr messenger RNA encoded by the humanized Ttr locus or measuring expression of a TTR protein encoded by the humanized Ttr locus after administering the one or more guide RNAs or the one or more DNAs encoding the one or more guide RNAs and before administering the human-TTR-targeting reagent. In one example, the human-TTR-targeting reagent is not administered until serum levels of the TTR protein encoded by the humanized Ttr locus are at least about 10 μg/mL, at least about 20 μg/mL, at least about 30 μg/mL, at least about 40 μg/mL, at least about 50 μg/mL, at least about 60 μg/mL, at least about 70 μg/mL, at least about 80 μg/mL, at least about 90 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/mL, at least about 500 μg/mL, at least about 600 μg/mL, at least about 700 μg/mL, at least about 800 μg/mL, at least about 900 μg/mL, or at least about 1000 μg/mL.

In another example, the human-TTR-targeting reagent is not administered until serum levels of the TTR protein encoded by the humanized Ttr locus are between about 10 μg/mL and about 20 μg/mL, between about 20 μg/mL and about 30 μg/mL, between about 30 μg/mL and about 40 μg/mL, between about 40 μg/mL and about 50 μg/mL, between about 50 μg/mL and about 60 μg/mL, between about 60 μg/mL and about 70 μg/mL, between about 70 μg/mL and about 80 μg/mL, between about 80 μg/mL and about 90 μg/mL, between about 90 μg/mL and about 100 μg/mL, between about 100 μg/mL and about 150 μg/mL, between about 150 μg/mL and about 200 μg/mL, between about 200 μg/mL and about 250 μg/mL, between about 250 μg/mL and about 300 μg/mL, between about 300 μg/mL and about 350 μg/mL, between about 350 μg/mL and about 400 μg/mL, between about 400 μg/mL and about 450 μg/mL, between about 450 μg/mL and about 500 μg/mL, between about 500 μg/mL and about 600 μg/mL, between about 600 μg/mL and about 700 μg/mL, between about 700 μg/mL and about 800 μg/mL, between about 800 μg/mL and about 900 μg/mL, or between about 900 μg/mL and about 1000 μg/mL.

In another example, the human-TTR-targeting reagent is not administered until serum levels of the TTR protein encoded by the humanized Ttr locus are between about 10 μg/mL and about 20 μg/mL, between about 10 μg/mL and about 30 μg/mL, between about 10 μg/mL and about 40 μg/mL, between about 10 μg/mL and about 50 μg/mL, between about 10 μg/mL and about 60 μg/mL, between about 10 μg/mL and about 70 μg/mL, between about 10 μg/mL and about 80 μg/mL, between about 10 μg/mL and about 90 μg/mL, between about 10 μg/mL and about 100 μg/mL, between about 10 μg/mL and about 150 μg/mL, between about 10 μg/mL and about 200 μg/mL, between about 10 μg/mL and about 250 μg/mL, between about 10 μg/mL and about 300 μg/mL, between about 10 μg/mL and about 350 μg/mL, between about 10 μg/mL and about 400 μg/mL, between about 10 μg/mL and about 450 μg/mL, between about 10 μg/mL and about 500 μg/mL, between about 10 μg/mL and about 600 μg/mL, between about 10 μg/mL and about 700 μg/mL, between about 10 μg/mL and about 800 μg/mL, between about 10 μg/mL and about 900 μg/mL, or between about 10 μg/mL and about 1000 μg/mL.

In another example, the human-TTR-targeting reagent is not administered until serum levels of the TTR protein encoded by the humanized Ttr locus are between about 10 μg/mL and about 1000 μg/mL, between about 20 μg/mL and about 1000 μg/mL, between about 30 μg/mL and about 1000 μg/mL, between about 40 μg/mL and about 1000 μg/mL, between about 50 μg/mL and about 1000 μg/mL, between about 60 μg/mL and about 1000 μg/mL, between about 70 μg/mL and about 1000 μg/mL, between about 80 μg/mL and about 1000 μg/mL, between about 90 μg/mL and about 1000 μg/mL, between about 100 μg/mL and about 1000 μg/mL, between about 150 μg/mL and about 1000 μg/mL, between about 200 μg/mL and about 1000 μg/mL, between about 250 μg/mL and about 1000 μg/mL, between about 300 μg/mL and about 1000 μg/mL, between about 350 μg/mL and about 1000 μg/mL, between about 400 μg/mL and about 1000 μg/mL, between about 450 μg/mL and about 1000 μg/mL, between about 500 μg/mL and about 1000 μg/mL, between about 600 μg/mL and about 1000 μg/mL, between about 700 μg/mL and about 1000 μg/mL, between about 800 μg/mL and about 1000 μg/mL, or between about 900 μg/mL and about 1000 μg/mL.

In another example, the human-TTR-targeting reagent is not administered until serum levels of the TTR protein encoded by the humanized Ttr locus are between about 10 μg/mL and about 450 μg/mL, between about 50 μg/mL and about 400 μg/mL, between about 100 μg/mL and about 350 μg/mL, between about 150 μg/mL and about 300 μg/mL, or between about 200 μg/mL and about 250 μg/mL.

Likewise, in some methods, the human-TTR-targeting reagent is not administered until TTR amyloid deposition occurs or is observed in the non-human animal following administering the guide RNAs or the DNA encoding the guide RNAs. The TTR amyloid deposition can be in any relevant tissue. Transthyretin amyloidosis is a slowly progressive condition characterized by the buildup of abnormal deposits of a protein called amyloid (amyloidosis) in the body's organs and tissues. These protein deposits most frequently occur in the peripheral nervous system, which is made up of nerves connecting the brain and spinal cord to muscles and sensory cells that detect sensations such as touch, pain, heat, and sound. Protein deposits in these nerves result in a loss of sensation in the extremities (peripheral neuropathy). The autonomic nervous system, which controls involuntary body functions such as blood pressure, heart rate, and digestion, may also be affected by amyloidosis. In some cases, the brain and spinal cord (central nervous system) are affected. Other areas of amyloidosis include the heart, kidneys, eyes, and gastrointestinal tract. In some methods, the human-TTR-targeting reagent is not administered until TTR amyloid deposition occurs or is observed in any one of or any combination of these areas, systems, or tissues.

The guide RNAs or the DNA encoding the guide RNAs can be administered (introduced into the cell or introduced into the animal such that the guide RNAs or the DNA gain access to the interior of cells in the non-human animal) in any form, in any delivery vehicle, and by any route of administration. For example, the administering the one or more guide RNAs or the one or more DNAs encoding the one or more guide RNAs can, in some methods, comprise adeno-associated virus (AAV)-mediated delivery, lipid nanoparticle (LNP)-mediated delivery, or hydrodynamic delivery (HDD). In one example, the guide RNAs or the DNA encoding the guide RNAs are administered via LNP-mediated delivery (e.g., at a dose between about 0.1 mg/kg to about 2 mg/kg). In another example, the guide RNAs or the DNA encoding the guide RNAs are administered via AAV-mediated delivery (e.g., using an AAV with a serotype for delivery to the liver, such as AAV8). The guide RNAs can be administered as RNA, or they can be administered as DNA. If administered as DNA, each guide-RNA-encoding sequence can be, in one example, operably linked to a different U6 promoter.

In some methods, the target sequences for the guide RNAs can comprise a regulatory sequence within the humanized Ttr locus. For example, the regulatory sequence can comprise a promoter or an enhancer. In some methods, the target sequences for the guide RNAs can be within 200 base pairs of the transcription start site of the genetically modified endogenous Ttr locus or can be within a region 200 base pairs upstream of the transcription start site and 1 base pair downstream of the transcription start site.

In some methods, the guide RNAs each comprise two adaptor-binding elements to which the chimeric adaptor protein can specifically bind. For example, a first adaptor-binding element can be within a first loop of each of the one or more guide RNAs, and a second adaptor-binding element can be within a second loop of each of the one or more guide RNAs. In a specific example, each guide RNA can be a single guide RNA comprising a CRISPR RNA (crRNA) portion fused to a transactivating CRISPR RNA (tracrRNA) portion, and the first loop is the tetraloop corresponding to residues 13-16 of SEQ ID NO: 146, 148, 150, or 151, and the second loop is the stem loop 2 corresponding to residues 53-56 of SEQ ID NO: 146, 148, 150, or 151. In another specific example, the adaptor-binding element comprises the sequence set forth in SEQ ID NO: 106. In another specific example, each of the one or more guide RNAs comprises the sequence set forth in SEQ ID NO: 127, 132, 140, or 141.

In one example, the guide RNAs can target a sequence comprising the sequence set forth in any one of SEQ ID NOS: 121-123. Likewise, the guide RNAs can comprise the sequence set forth in any one of SEQ ID NOS: 124-126.

In some methods, the one or more guide RNAs comprise multiple guide RNAs that target the humanized Ttr locus (e.g., at least two or at least three guide RNAs that target the humanized Ttr locus). In a specific example, a first guide RNA targets a sequence comprising SEQ ID NO: 121 or comprises the sequence set forth in SEQ ID NO: 124, a second guide RNA targets a sequence comprising SEQ ID NO: 122 or comprises the sequence set forth in SEQ ID NO: 125, and a third guide RNA targets a sequence comprising SEQ ID NO: 123 or comprises the sequence set forth in SEQ ID NO: 126.

The human-TTR-targeting reagent can be a human-TTR-targeting antibody or antigen-binding protein or any other large molecule or small molecule that targets human TTR protein. Alternatively, the human-TTR-targeting reagent can be any biological or chemical agent that targets the human TTR locus (the human TTR gene), the human TTR mRNA, or the human TTR protein. Examples of human-TTR-targeting reagents are disclosed elsewhere herein.

Such human-TTR-targeting reagents can be administered by any delivery method/vehicle (e.g., AAV, LNP, HDD, or injection) and by any route of administration. Means of delivering complexes and molecules and routes of administration are disclosed in more detail elsewhere herein. In particular methods, the reagents delivered via AAV-mediated delivery. For example, AAV8 can be used to target the liver. In other particular methods, the reagents are delivered by LNP-mediated delivery. In other particular methods, the reagents are delivered by hydrodynamic delivery (HDD). The dose can be any suitable dose.

Methods for assessing activity of the human-TTR-targeting reagent are well-known and are provided elsewhere herein. Assessment of activity can be in any cell type, any tissue type, or any organ type as disclosed elsewhere herein. In some methods, assessment of activity is in liver cells or in the liver.

If the human-TTR-targeting reagent is a genome editing reagent (e.g., a nuclease agent), such methods can comprise assessing modification of the humanized TTR locus. As one example, the assessing can comprise measuring non-homologous end joining (NHEJ) activity at the humanized TTR locus. This can comprise, for example, measuring the frequency of insertions or deletions within the humanized TTR locus. For example, the assessing can comprise sequencing the humanized TTR locus in one or more cells isolated from the non-human animal (e.g., next-generation sequencing). Assessment can comprise isolating a target organ or tissue (e.g., liver) from the non-human animal and assessing modification of humanized TTR locus in the target organ or tissue. Assessment can also comprise assessing modification of humanized TTR locus in two or more different cell types within the target organ or tissue. Similarly, assessment can comprise isolating a non-target organ or tissue (e.g., two or more non-target organs or tissues) from the non-human animal and assessing modification of humanized TTR locus in the non-target organ or tissue.

Such methods can also comprise measuring expression levels of the mRNA produced by the humanized TTR locus, or by measuring expression levels of the protein encoded by the humanized TTR locus. For example, protein levels can be measured in a particular cell, tissue, or organ type (e.g., liver), or secreted levels can be measured in the serum. Methods for assessing expression of TTR mRNA or TTR protein expressed from the humanized TTR locus are provided elsewhere herein and are well-known.

As one specific example, if the human-TTR-targeting reagent is a genome editing reagent (e.g., a nuclease agent), percent editing (e.g., total number of insertions or deletions observed over the total number of sequences read in the PCR reaction from a pool of lysed cells) at the humanized TTR locus can be assessed (e.g., in liver cells).

The various methods provided above for assessing activity in vivo can also be used to assess the activity of human-TTR-targeting reagents ex vivo (e.g., in a liver comprising a humanized TTR locus) or in vitro (e.g., in a cell comprising a humanized TTR locus) as described elsewhere herein.

In some methods, the human-TTR-targeting reagent is a nuclease agent, such as a CRISPR/Cas nuclease agent, that targets the human TTR gene. Such methods can comprise, for example: (a) introducing into the non-human animal a nuclease agent (or a nucleic acid encoding the nuclease agent) designed to cleave the human TTR gene (e.g., Cas protein such as Cas9 (or a nucleic acid encoding Cas9) and a guide RNA (or a DNA encoding the guide RNA) designed to target a guide RNA target sequence in the human TTR gene); and (b) assessing modification of the humanized TTR locus comprising the V30M mutation.

In the case of a CRISPR/Cas nuclease, for example, modification of the humanized TTR locus comprising the V30M mutation will be induced when the guide RNA forms a complex with the Cas protein and directs the Cas protein to the humanized TTR locus, and the Cas/guide RNA complex cleaves the guide RNA target sequence, triggering repair by the cell (e.g., via non-homologous end joining (NHEJ) if no donor sequence is present).

Optionally, two or more guide RNAs (or DNAs encoding the guide RNAs) can be introduced, each designed to target a different guide RNA target sequence within the human TTR gene. For example, two guide RNAs can be designed to excise a genomic sequence between the two guide RNA target sequences. Modification of the humanized TTR locus will be induced when the first guide RNA forms a complex with the Cas protein and directs the Cas protein to the humanized TTR locus, the second guide RNA forms a complex with the Cas protein and directs the Cas protein to the humanized TTR locus, the first Cas/guide RNA complex cleaves the first guide RNA target sequence, and the second Cas/guide RNA complex cleaves the second guide RNA target sequence, resulting in excision of the intervening sequence.

Optionally, an exogenous donor nucleic acid capable of recombining with and modifying a human TTR gene is also introduced into the non-human animal. Optionally, the nuclease agent or Cas protein can be tethered to the exogenous donor nucleic acid as described elsewhere herein. Modification of the humanized TTR locus will be induced, for example, when the guide RNA forms a complex with the Cas protein and directs the Cas protein to the humanized TTR locus, the Cas/guide RNA complex cleaves the guide RNA target sequence, and the humanized TTR locus recombines with the exogenous donor nucleic acid to modify the humanized TTR locus. The humanized TTR locus can then be repaired with the exogenous donor nucleic acid, for example, via homology-directed repair (HDR) or via NHEJ-mediated insertion. Any type of exogenous donor nucleic acid can be used, examples of which are provided elsewhere herein.

Some methods comprise administering exogenous, pre-formed TTR aggregates or fibrils to the non-human animal prior to or simultaneously with introducing the human-TTR-targeting reagent. For example, the administering of the exogenous, pre-formed TTR aggregates or fibrils can be at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, or at least 12 months prior to introducing the human-TTR-targeting reagent. Alternatively, the administering of the exogenous, pre-formed TTR aggregates or fibrils can be no more than 1 day, no more than 2 days, no more than 3 days, no more than 4 days, no more than 5 days, no more than 6 days, no more than 1 week, no more than 2 weeks, no more than 3 weeks, no more than 4 weeks, no more than 1 month, no more than 2 months, no more than 3 months, no more than 4 months, no more than 5 months, no more than 6 months, no more than 7 months, no more than 8 months, no more than 9 months, no more than 10 months, no more than 11 months, or no more than 12 months prior to introducing the human-TTR-targeting reagent. Alternatively, the administering of the exogenous, pre-formed TTR aggregates or fibrils can be between 1 day and 12 months, between 1 week and 12 months, between 1 month and 12 months, between 2 months and 12 months, between 3 months and 12 months, between 4 months and 12 months, between 5 months and 12 months, between 6 months and 12 months, between 1 day and 6 months, between 1 day and 5 months, between 1 day and 4 months, between 1 day and 3 months, between 1 day and 2 months, between 1 day and 1 month, between 1 day and 4 weeks, between 1 day and 3 weeks, between 1 day and 2 weeks, or between 1 day and 1 week prior to introducing the human-TTR-targeting reagent.

The exogenous, pre-formed TTR aggregates or fibrils can be administered to the non-human animal one time or multiple times. For example, they can be administered at least 2, at least 3, at least 4, at least 5, at least 6, at least 7, at least 8, at least 9, or at least 10 times. Alternatively, they can be administered no more than 2, no more than 3, no more than 4, no more than 5, no more than 6, no more than 7, no more than 8, no more than 9, or no more than 10 times. Alternatively, they can be administered between 1 and 10 times, between 2 and 10 times, between 3 and 10 times, between 4 and 10 times, between 5 and 10 times, between 1 and 9 times, between 1 and 8 times, between 1 and 7 times, between 1 and 6 times, between 1 and 5 times, between 1 and 4 times, or between 1 and 3 times.

The pre-formed TTR aggregates or fibrils can be V30M TTR aggregates or fibrils, can be wild type TTR aggregates or fibrils, or can be TTR aggregates or fibrils in which the TTR comprises a mutation other than or in addition to V30M. Likewise, the pre-formed TTR aggregates or fibrils can be human TTR aggregates or fibrils (e.g., human TTR V30M aggregates or fibrils) or can be mouse TTR aggregates or fibrils.

The pre-formed TTR aggregates or fibrils can be administered via any suitable route. For example, the pre-formed TTR aggregates or fibrils can be injected via intravenous injection (e.g., tail vein injection). For example, the pre-formed TTR aggregate or fibrils can be administered via hydrodynamic delivery. In some cases, the TTR aggregates or fibrils can be administered together with heparin (i.e., exogenous heparin), which can serve as a template for amyloid fibrils to form and accelerate TTR amyloid deposition.

B. Methods of Optimizing Delivery or Efficacy of Hum an-TTR-Targeting Reagent In Vivo or Ex Vivo

Various methods are provided for optimizing delivery of human-TTR-targeting reagents to a cell or non-human animal or optimizing the activity or efficacy of human-TTR-targeting reagents in vivo. Such methods can comprise, for example: (a) performing the method of testing the efficacy of a human-TTR-targeting reagent as described above a first time in a first non-human animal or first cell; (b) changing a variable and performing the method a second time in a second non-human animal (i.e., of the same species) or a second cell with the changed variable; and (c) comparing the activity of the human-TTR-targeting reagent in step (a) with the activity of the human-TTR-targeting reagent in step (b), and selecting the method resulting in the higher activity.

Methods of measuring delivery, efficacy, or activity of human-TTR-targeting reagents are disclosed elsewhere herein. For example, such methods can comprise measuring modification of the humanized TTR locus comprising the V30M mutation. More effective modification of the humanized TTR locus can mean different things depending on the desired effect within the non-human animal or cell. For example, more effective modification of the humanized TTR locus can mean one or more or all of higher levels of modification, higher precision, higher consistency, or higher specificity. Higher levels of modification (i.e., higher efficacy) of the humanized TTR locus refers to a higher percentage of cells is targeted within a particular target cell type, within a particular target tissue, or within a particular target organ (e.g., liver). Higher precision refers to more precise modification of the humanized TTR locus (e.g., a higher percentage of targeted cells having the same modification or having the desired modification without extra unintended insertions and deletions (e.g., NHEJ indels)). Higher consistency refers to more consistent modification of the humanized TTR locus among different types of targeted cells, tissues, or organs if more than one type of cell, tissue, or organ is being targeted (e.g., modification of a greater number of cell types within the liver). If a particular organ is being targeted, higher consistency can also refer to more consistent modification throughout all locations within the organ (e.g., the liver). Higher specificity can refer to higher specificity with respect to the genomic locus or loci targeted, higher specificity with respect to the cell type targeted, higher specificity with respect to the tissue type targeted, or higher specificity with respect to the organ targeted. For example, increased genomic locus specificity refers to less modification of off-target genomic loci (e.g., a lower percentage of targeted cells having modifications at unintended, off-target genomic loci instead of or in addition to modification of the target genomic locus). Likewise, increased cell type, tissue, or organ type specificity refers to less modification of off-target cell types, tissue types, or organ types if a particular cell type, tissue type, or organ type is being targeted (e.g., when a particular organ is targeted (e.g., the liver), there is less modification of cells in organs or tissues that are not intended targets).

Alternatively, such methods can comprise measuring expression of TTR mRNA or TTR protein. In one example, a more effective human-TTR-targeting agent results in a greater decrease in TTR mRNA or TTR protein expression. Alternatively, such methods can comprise measuring TTR activity. In one example, a more effective human-TTR-targeting agent results in a greater decrease in TTR activity.

The variable that is changed can be any parameter. As one example, the changed variable can be the packaging or the delivery method/vehicle by which the human-TTR-targeting reagent or reagents are introduced into the cell or non-human animal. Examples of delivery methods/vehicles, such as LNP, HDD, and AAV, are disclosed elsewhere herein. For example, the changed variable can be the AAV serotype. Alternatively, the changed variable can be the dose of AAV delivered (e.g., about 10¹¹, about 10¹², about 10¹³, or about 10¹⁴ vg/kg of body weight). Similarly, the administering can comprise LNP-mediated delivery, and the changed variable can be the LNP formulation. Alternatively, the administering can comprise LNP-mediated delivery, and the changed variable can be the dose of the LNP delivered (e.g., about 0.01 mg/kg, about 0.03 mg/kg, about 0.1 mg/kg, about 0.3 mg/kg, about 1 mg/kg, about 3 mg/kg, or about 10 mg/kg). As another example, the changed variable can be the route of administration for introduction of the human-TTR-targeting reagent or reagents into the cell or non-human animal. Examples of routes of administration, such as intravenous, intravitreal, intraparenchymal, and nasal instillation, are disclosed elsewhere herein.

As another example, the changed variable can be the concentration or amount of the human-TTR-targeting reagent or reagents introduced. As another example, the changed variable can be the concentration or the amount of one human-TTR-targeting reagent introduced (e.g., guide RNA, Cas protein, exogenous donor nucleic acid, RNAi agent, or ASO) relative to the concentration or the amount another human-TTR-targeting reagent introduced (e.g., guide RNA, Cas protein, exogenous donor nucleic acid, RNAi agent, or ASO).

As another example, the changed variable can be the timing of introducing the human-TTR-targeting reagent or reagents relative to the timing of assessing the activity or efficacy of the reagents. As another example, the changed variable can be the number of times or frequency with which the human-TTR-targeting reagent or reagents are introduced. As another example, the changed variable can be the timing of introduction of one human-TTR-targeting reagent introduced (e.g., guide RNA, Cas protein, exogenous donor nucleic acid, RNAi agent, or ASO) relative to the timing of introduction of another human-TTR-targeting reagent introduced (e.g., guide RNA, Cas protein, exogenous donor nucleic acid, RNAi agent, or ASO).

As another example, the changed variable can be the form in which the human-TTR-targeting reagent or reagents are introduced. For example, a guide RNA can be introduced in the form of DNA or in the form of RNA. A Cas protein (e.g., Cas9) can be introduced in the form of DNA, in the form of RNA, or in the form of a protein (e.g., complexed with a guide RNA). An exogenous donor nucleic acid can be DNA, RNA, single-stranded, double-stranded, linear, circular, and so forth. Similarly, each of the components can comprise various combinations of modifications for stability, to reduce off-target effects, to facilitate delivery, and so forth. Likewise, RNAi agents and ASOs, for example, can comprise various combinations of modifications for stability, to reduce off-target effects, to facilitate delivery, and so forth.

As another example, the changed variable can be the human-TTR-targeting reagent or reagents that are introduced. For example, if the human-TTR-targeting reagent comprises a guide RNA, the changed variable can be introducing a different guide RNA with a different sequence (e.g., targeting a different guide RNA target sequence). Similarly, if the human-TTR-targeting reagent comprises an RNAi agent or an ASO, the changed variable can be introducing a different RNAi agent or ASO with a different sequence. Likewise, if the human-TTR-targeting reagent comprises a Cas protein, the changed variable can be introducing a different Cas protein (e.g., introducing a different Cas protein with a different sequence, or a nucleic acid with a different sequence (e.g., codon-optimized) but encoding the same Cas protein amino acid sequence). Likewise, if the human-TTR-targeting reagent comprises an exogenous donor nucleic acid, the changed variable can be introducing a different exogenous donor nucleic acid with a different sequence (e.g., a different insert nucleic acid or different homology arms (e.g., longer or shorter homology arms or homology arms targeting a different region of the human TTR gene)).

In a specific example, the human-TTR-targeting reagent comprises a Cas protein and a guide RNA designed to target a guide RNA target sequence in a human TTR gene. In such methods, the changed variable can be the guide RNA sequence and/or the guide RNA target sequence. In some such methods, the Cas protein and the guide RNA can each be administered in the form of RNA, and the changed variable can be the ratio of Cas mRNA to guide RNA (e.g., in an LNP formulation). In some such methods, the changed variable can be guide RNA modifications (e.g., a guide RNA with a modification is compared to a guide RNA without the modification).

In another specific example, the human-TTR-targeting reagent comprises an RNAi agent or ASO agent targeting human TTR. In such methods, the changed variable can be the RNAi agent or ASO agent sequence and/or the RNAi agent or ASO agent target sequence. In some such methods, the changed variable can be the RNAi agent or ASO agent modification pattern.

C. Human-TTR-Targeting Reagents

A human-TTR-targeting reagent can be any reagent that targets a human TTR protein, a human TTR gene, or a human TTR mRNA. A human-TTR-targeting reagent can be, for example, a known human-TTR-targeting reagent, can be a putative human-TTR-targeting reagent (e.g., candidate reagents designed to target human TTR), or can be a reagent being screened for human-TTR-targeting activity.

For example, a human-TTR-targeting reagent can be an antigen-binding protein (e.g., agonist antibody) targeting an epitope of a human TTR protein. The term “antigen-binding protein” includes any protein that binds to an antigen. Examples of antigen-binding proteins include an antibody, an antigen-binding fragment of an antibody, a multispecific antibody (e.g., a bi-specific antibody), an scFV, a bis-scFV, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes). Other human-TTR-targeting reagents include small molecules targeting a human TTR protein.

Other human-TTR-targeting reagents can include genome editing reagents such as a nuclease agent (e.g., a Clustered Regularly Interspersed Short Palindromic Repeats (CRISPR)/CRISPR-associated (Cas) (CRISPR/Cas) nuclease, a zinc finger nuclease (ZFN), or a Transcription Activator-Like Effector Nuclease (TALEN)) that cleaves a recognition site within the human TTR gene. Likewise, a human-TTR-targeting reagent can be an exogenous donor nucleic acid (e.g., a targeting vector or single-stranded oligodeoxynucleotide (ssODN)) designed to recombine with the human TTR gene.

Other human-TTR-targeting reagents can include RNAi agents. An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to: short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Other human-TTR-targeting reagents can include antisense oligonucleotides (ASOs). Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H. Examples of human-TTR-targeting RNAi agents or antisense oligonucleotides are known. See, e.g., Ackermann et al. (2012) Amyloid Suppl 1:43-44 and Coelho et al. (2013) N. Engl. J. Med. 369(9):819-829, each of which is herein incorporated by reference in its entirety for all purposes.

Other human-TTR-targeting reagents include small-molecule reagents. One example of such a small-molecule reagent is tafamidis, which functions by kinetic stabilization of the correctly folded tetrameric form of the transthyretin (TTR) protein. See, e.g., Hammarstrom et al. (2003) Science 299:713-716, herein incorporated by reference in its entirety for all purposes.

D. Administering Human-TTR-Targeting Reagents and/or SAM Guide RNAs and/or Recombinase Expression Cassettes to Non-Human Animals or Cells

The methods disclosed herein can comprise introducing into a non-human animal or cell various molecules (e.g., human-TTR-targeting reagents such as therapeutic molecules or complexes and/or SAM guide RNAs as described herein or DNA encoding SAM guide RNAs and/or recombinases or nucleic acids encoding recombinases), including nucleic acids, proteins, nucleic-acid-protein complexes, protein complexes, or small molecules. “Introducing” includes presenting to the cell or non-human animal the molecule (e.g., nucleic acid or protein) in such a manner that it gains access to the interior of the cell or to the interior of cells within the non-human animal. The introducing can be accomplished by any means, and two or more of the components (e.g., two of the components, or all of the components) can be introduced into the cell or non-human animal simultaneously or sequentially in any combination. For example, a Cas protein can be introduced into a cell or non-human animal before introduction of a guide RNA, or it can be introduced following introduction of the guide RNA. As another example, an exogenous donor nucleic acid can be introduced prior to the introduction of a Cas protein and a guide RNA, or it can be introduced following introduction of the Cas protein and the guide RNA (e.g., the exogenous donor nucleic acid can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or non-human animal by the same delivery method/vehicle or different delivery methods/vehicles. Similarly, two or more of the components can be introduced into a non-human animal by the same route of administration or different routes of administration.

In some methods, components of a CRISPR/Cas system are introduced into a non-human animal or cell. A guide RNA can be introduced into a non-human animal or cell in the form of an RNA (e.g., in vitro transcribed RNA) or in the form of a DNA encoding the guide RNA. When introduced in the form of a DNA, the DNA encoding a guide RNA can be operably linked to a promoter active in a cell in the non-human animal. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a rodent cell, a mouse cell, a rat cell, or any other host cell of interest, as compared to the naturally occurring polynucleotide sequence. When a nucleic acid encoding the Cas protein is introduced into a non-human animal, the Cas protein can be transiently, conditionally, or constitutively expressed in a cell in the non-human animal.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery.

Molecules (e.g., Cas proteins or guide RNAs or RNAi agents or ASOs) introduced into the non-human animal or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or non-human animal. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4): 1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, N.Y.: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359.

Other methods for introducing molecules (e.g., nucleic acid or proteins) into a cell or non-human animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., adeno-associated virus (AAV)-mediated delivery), and lipid-nanoparticle-mediated delivery.

Introduction of nucleic acids and proteins into cells or non-human animals can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Exemplary viral titers (e.g., AAV titers) include 10¹², 10¹³, 10¹⁴, 10¹⁵, and 10¹⁶ vector genomes/mL. Other exemplary viral titers (e.g., AAV titers) include about 10¹², about 10¹³, about 10¹⁴, about 10¹⁵, and about 10¹⁶ vector genomes(vg)/kg of body weight.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. Delivery through such methods results in transient Cas expression, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1, herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Reports 22:1-9 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Reports 22:1-9 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), di stearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine di stearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA to gRNA nucleic acid of from about 1:1 to about 1:2. In specific examples, the ratio of Cas mRNA to gRNA can be about 1:1 or about 1:2.

Exemplary dosing of LNPs includes, for example, about 0.1, about 0.25, about 0.3, about 0.5, about 1, about 2, about 3, about 4, about 5, about 6, about 8, or about 10 mg/kg (mpk) with respect to total RNA (e.g., Cas9 mRNA and gRNA) cargo content. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 1:1, 1:3, 1:5, 1:10, or 1:25.

A specific example of a suitable LNP has a nitrogen-to-phosphate (N/P) ratio of 4.5 and contains biodegradable cationic lipid, cholesterol, DSPC, and PEG2k-DMG in a 45:44:9:2 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Reports 22:1-9, herein incorporated by reference in its entirety for all purposes. The Cas9 mRNA can be in a 1:1 ratio by weight to the guide RNA. Another specific example of a suitable LNP contains Dlin-MC3-DMA (MC3), cholesterol, DSPC, and PEG-DMG in a 50:38.5:10:1.5 molar ratio.

Another specific example of a suitable LNP has a nitrogen-to-phosphate (N/P) ratio of 6 and contains biodegradable cationic lipid, cholesterol, DSPC, and PEG2k-DMG in a 50:38:9:3 molar ratio. The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. The Cas9 mRNA can be in a 1:2 ratio by weight to the guide RNA.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or exogenous donor nucleic acid/repair template). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyms or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions comprising the guide RNAs and/or Cas proteins (or nucleic acids encoding the guide RNAs and/or Cas proteins) can be formulated using one or more physiologically and pharmaceutically acceptable carriers, diluents, excipients, or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the exogenous donor nucleic acids, guide RNAs, or Cas proteins (or nucleic acids encoding the guide RNAs or Cas proteins) and the route of administration among other factors. The introduction of nucleic acids or proteins into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

E. Measuring Delivery, Activity, or Efficacy of Human-TTR-Targeting Reagents In Vivo or Ex Vivo

The methods disclosed herein can further comprise detecting or measuring activity of human-TTR-targeting reagents.

If the human-TTR-targeting reagent is a genome editing reagent (e.g., CRISPR/Cas designed to target the human TTR locus), the measuring can comprise assessing the humanized TTR locus comprising the V30M mutation for modifications. Various methods can be used to identify cells having a targeted genetic modification. The screening can comprise a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, herein incorporated by reference in its entirety for all purposes). Next-generation sequencing (NGS) can also be used for screening. Next-generation sequencing can also be referred to as “NGS” or “massively parallel sequencing” or “high throughput sequencing.” NGS can be used as a screening tool in addition to the MOA assays to define the exact nature of the targeted genetic modification and whether it is consistent across cell types or tissue types or organ types.

Assessing modification of the humanized TTR locus comprising the V30M mutation in a non-human animal can be in any cell type from any tissue or organ. For example, the assessment can be in multiple cell types from the same tissue or organ or in cells from multiple locations within the tissue or organ. This can provide information about which cell types within a target tissue or organ are being targeted or which sections of a tissue or organ are being reached by the human-TTR-targeting reagent. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

If the reagent is designed to inactivate the humanized TTR locus comprising the V30M mutation, affect expression of the humanized TTR locus, prevent translation of the humanized TTR mRNA, or clear the humanized TTR protein, the measuring can comprise assessing humanized TTR mRNA or protein expression. This measuring can be within the liver or particular cell types or regions within the liver, or it can involve measuring serum levels of secreted humanized TTR protein.

Production and secretion of the humanized TTR protein comprising the V30M mutation can be assessed by any known means. For example, expression can be assessed by measuring levels of the encoded mRNA in the liver of the non-human animal or levels of the encoded protein in the liver of the non-human animal using known assays. Secretion of the humanized TTR protein can be assessed by measuring or plasma levels or serum levels of the encoded humanized TTR protein in the non-human animal using known assays. For example, the measuring can be to determine if the human-TTR-targeting reagent reduces TTR levels in the non-human animal.

TTR amyloid deposition or the presence of TTR aggregates or fibrils can also be assessed by known means, and other phenotypes such as neuropathy or peripheral neuropathy or TTR amyloid neuropathy or polyneuropathy (e.g., TTR amyloid deposits around peripheral nerves) can be assessed by known means.

The assessing in a non-human animal can be in any cell type from any tissue or organ. For example, the assessment can be in multiple cell types from the same tissue or organ (e.g., liver) or in cells from multiple locations within the tissue or organ. This can provide information about which cell types within a target tissue or organ are being targeted or which sections of a tissue or organ are being reached by the human-TTR-targeting reagent. As another example, the assessment can be in multiple types of tissue or in multiple organs. In methods in which a particular tissue, organ, or cell type is being targeted, this can provide information about how effectively that tissue or organ is being targeted and whether there are off-target effects in other tissues or organs.

One example of an assay that can be used are the RNASCOPE™ and BASESCOPE™ RNA in situ hybridization (ISH) assays, which are methods that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a ZZ probe, and it can differentially detect single nucleotide edits and mutations in intact fixed tissue.

The assessment of any of these phenotypes can be at any age of non-human animal, such as at least about 1, 2, 3, 4, 5, 6, 7, 8, 9, 10, 11, or 12 months of age.

The assessment of any of these phenotypes can be done in comparison to a control non-human animal. One example of a control non-human animal is a corresponding wild type animal (e.g., of the same species). For example, the control non-human animal can be a wild type littermate. Another example of a control non-human animal is a corresponding non-human animal comprising a humanized TTR locus without the V30M mutation (e.g., the humanized TTR locus is identical except for the absence of the V30M mutation). The control non-human animals can be, for example, the same age as the test non-human animal and/or the same sex as the test non-human animal. The assessment of any of these phenotypes can also be done in comparison to a control non-human animal that is identical to the test non-human animal except not treated with the human-TTR-targeting reagent.

The assessment of any of these phenotypes can be in a single non-human animal and assessing changes in that non-human animal. Alternatively, the assessment can be in a population of non-human animals and comparing, for example, the percentage of non-human animals having a particular phenotype.

F. Measuring CRISPR/Cas-Induced Upregulation of Expression of Humanized TTR Locus In Vivo

The methods disclosed herein can further comprise assessing expression of the humanized TTR locus or upregulation of the humanized TTR locus by the synergistic activation mediator (SAM) systems disclosed herein.

For example, the method of assessing expression can comprise measuring expression or activity of the encoded TTR mRNA and/or TTR protein. For example, serum levels of the encoded TTR protein can be measured. Assays for measuring levels and activity of RNA and proteins are well known.

Assessing expression of the humanized TTR locus in a non-human animal can be in any cell type from any tissue or organ. For example, expression of the humanized TTR locus can be assessed in multiple cell types from the same tissue or organ or in cells from multiple locations within the tissue or organ. This can provide information about which cell types within a target tissue or organ are being targeted or which sections of a tissue or organ are being reached by the CRISPR/Cas and modified. As another example, expression of the humanized TTR locus can be assessed in multiple types of tissue or in multiple organs. In methods in which a particular tissue or organ is being targeted, this can provide information about how effectively that tissue or organ is being targeted and whether there are off-target effects in other tissues or organs.

G. Methods of Accelerating TTR Amyloid Deposition

Also provided are methods of accelerating TTR amyloid deposition in the non-human animals or non-human animal cells disclosed herein (i.e., the non-human animals or cells comprising a humanized TTR locus comprising a V30M mutation). Such methods can comprise administering exogenous, pre-formed TTR aggregates or fibrils to the non-human animal. The exogenous, pre-formed TTR aggregates or fibrils can be administered to the non-human animal one time or multiple times. For example, they can be administered at least 2, at least 3, at least 4, at least 5, at least 6, at least 7, at least 8, at least 9, or at least 10 times. Alternatively, they can be administered no more than 2, no more than 3, no more than 4, no more than 5, no more than 6, no more than 7, no more than 8, no more than 9, or no more than 10 times. Alternatively, they can be administered between 1 and 10 times, between 2 and 10 times, between 3 and 10 times, between 4 and 10 times, between 5 and 10 times, between 1 and 9 times, between 1 and 8 times, between 1 and 7 times, between 1 and 6 times, between 1 and 5 times, between 1 and 4 times, or between 1 and 3 times.

The pre-formed TTR aggregates or fibrils can be V30M TTR aggregates or fibrils, can be wild type TTR aggregates or fibrils, or can be TTR aggregates or fibrils in which the TTR comprises a mutation other than or in addition to V30M. Likewise, the pre-formed TTR aggregates or fibrils can be human TTR aggregates or fibrils (e.g., human TTR V30M aggregates or fibrils) or can be mouse TTR aggregates or fibrils.

The pre-formed TTR aggregates or fibrils can be administered via any suitable route. For example, the pre-formed TTR aggregates or fibrils can be injected via intravenous injection (e.g., tail vein injection). For example, the pre-formed TTR aggregate or fibrils can be administered via hydrodynamic delivery. In some cases, the TTR aggregates or fibrils can be administered together with heparin (i.e., exogenous heparin), which can serve as a template for amyloid fibrils to form and accelerate TTR amyloid deposition.

V. Methods of Making Non-Human Animals Comprising a Humanized TTR Locus Comprising a V30M Mutation

Various methods are provided for making a non-human animal genome, non-human animal cell, or non-human animal comprising a humanized TTR locus comprising a V30M mutation as disclosed elsewhere herein. Likewise, various methods are provided for making a humanized TTR gene or locus comprising the V30M mutation or for making a non-human animal genome or non-human animal cell comprising a humanized TTR locus comprising the V30M mutation as disclosed elsewhere herein. Likewise, various methods are provided for making a non-human animal comprising a synergistic activation mediator (SAM) expression cassette (comprising a chimeric Cas protein coding sequence and a chimeric adaptor protein expression coding sequence) and a humanized TTR locus comprising a V30M mutation as disclosed elsewhere herein. Any convenient method or protocol for producing a genetically modified organism is suitable for producing such a genetically modified non-human animal. See, e.g., Poueymirou et al. (2007) Nat. Biotechnol. 25(1):91-99; U.S. Pat. Nos. 7,294,754; 7,576,259; 7,659,442; 8,816,150; 9,414,575; 9,730,434; and 10,039,269, each of which is herein incorporated by reference in its entirety for all purposes (describing mouse ES cells and the VELOCIMOUSE® method for making a genetically modified mouse). See also US 2014/0235933 A1, US 2014/0310828 A1, each of which is herein incorporated by reference in its entirety for all purposes (describing rat ES cells and methods for making a genetically modified rat). See also Cho et al. (2009) Curr. Protoc. Cell. Biol. 42:19.11.1-19.11.22 (doi: 10.1002/0471143030.cb1911s42) and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted TTR locus.

For example, the method of producing a non-human animal comprising a humanized TTR locus comprising the V30M mutation can comprise: (1) providing a pluripotent cell (e.g., an embryonic stem (ES) cell such as a mouse ES cell or a rat ES cell) comprising the humanized TTR locus comprising the V30M mutation; (2) introducing the genetically modified pluripotent cell into a non-human animal host embryo; and (3) gestating the host embryo in a surrogate mother.

As another example, the method of producing a non-human animal comprising a humanized TTR locus comprising the V30M mutation can comprise: (1) modifying the genome of a pluripotent cell (e.g., an embryonic stem (ES) cell such as a mouse ES cell or a rat ES cell) to comprise the humanized TTR locus comprising the V30M mutation; (2) identifying or selecting the genetically modified pluripotent cell comprising the humanized TTR locus comprising the V30M mutation; (3) introducing the genetically modified pluripotent cell into a non-human animal host embryo; and (4) gestating the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized TTR locus comprising the V30M mutation (and capable of transmitting the genetic modification through the germline).

Alternatively, the method of producing the non-human animals described elsewhere herein can comprise: (1) modifying the genome of a one-cell stage embryo to comprise the humanized TTR locus comprising the V30M mutation using the methods described above for modifying pluripotent cells; (2) selecting the genetically modified embryo; and (3) gestating the genetically modified embryo in a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The modified cell or one-cell stage embryo can be generated, for example, through recombination by (a) introducing into the cell one or more exogenous donor nucleic acids (e.g., targeting vectors) comprising an insert nucleic acid flanked, for example, by 5′ and 3′ homology arms corresponding to 5′ and 3′ target sites (e.g., target sites flanking the endogenous sequences intended for deletion and replacement with the insert nucleic acid), wherein the insert nucleic acid comprises a human TTR sequence and the V30M mutation to generate a humanized TTR locus comprising the V30M mutation; and (b) identifying at least one cell comprising in its genome the insert nucleic acid integrated at the endogenous TTR locus (i.e., identifying at least one cell comprising the humanized TTR locus comprising the V30M mutation). Likewise, a modified non-human animal genome or humanized non-human animal TTR gene comprising the V30M mutation can be generated, for example, through recombination by (a) contacting the genome or gene with one or more exogenous donor nucleic acids (e.g., targeting vectors) comprising 5′ and 3′ homology arms corresponding to 5′ and 3′ target sites (e.g., target sites flanking the endogenous sequences intended for deletion and replacement with an insert nucleic acid (e.g., comprising a human TTR sequence and the V30M mutation to generate a humanized TTR locus comprising the V30M mutation) flanked by the 5′ and 3′ homology arms), wherein the exogenous donor nucleic acids are designed for humanization of the endogenous non-human animal TTR locus.

Alternatively, the modified pluripotent cell or one-cell stage embryo can be generated by (a) introducing into the cell: (i) a nuclease agent, wherein the nuclease agent induces a nick or double-strand break at a target site within the endogenous TTR locus; and (ii) one or more exogenous donor nucleic acids (e.g., targeting vectors) comprising an insert nucleic acid flanked by, for example, 5′ and 3′ homology arms corresponding to 5′ and 3′ target sites (e.g., target sites flanking the endogenous sequences intended for deletion and replacement with the insert nucleic acid), wherein the insert nucleic acid comprises a human TTR sequence and the V30M mutation to generate a humanized TTR locus comprising the V30M mutation; and (c) identifying at least one cell comprising in its genome the insert nucleic acid integrated at the endogenous TTR locus (i.e., identifying at least one cell comprising the humanized TTR locus comprising the V30M mutation). Likewise, a humanized non-human animal genome or humanized non-human animal TTR gene comprising the V30M mutation can be generated by contacting the genome or gene with: (i) a nuclease agent, wherein the nuclease agent induces a nick or double-strand break at a target site within the endogenous TTR locus or gene; and (ii) one or more exogenous donor nucleic acids (e.g., targeting vectors) comprising an insert nucleic acid (e.g., comprising a human TTR sequence and the V30M mutation to generate a humanized TTR locus comprising the V30M mutation) flanked by, for example, 5′ and 3′ homology arms corresponding to 5′ and 3′ target sites (e.g., target sites flanking the endogenous sequences intended for deletion and replacement with the insert nucleic acid), wherein the exogenous donor nucleic acids are designed for humanization of the endogenous TTR locus and introduction of the V30M mutation. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA. In another example, the nuclease comprises a Cas9 protein and two or more, three or more, or four or more guide RNAs.

The step of modifying the genome can, for example, utilize exogenous repair templates (e.g., targeting vectors) to modify a TTR locus to comprise a humanized TTR locus comprising a V30M mutation disclosed herein. As one example, the targeting vector can be for generating a humanized TTR gene comprising the V30M mutation at an endogenous TTR locus (e.g., endogenous non-human animal TTR locus), wherein the targeting vector comprises a nucleic acid insert comprising human TTR sequence and the V30M mutation to be integrated in the TTR locus flanked by a 5′ homology arm targeting a 5′ target sequence at the endogenous TTR locus and a 3′ homology arm targeting a 3′ target sequence at the endogenous TTR locus. Integration of a nucleic acid insert in the TTR locus can result in addition of a nucleic acid sequence of interest in the TTR locus, deletion of a nucleic acid sequence of interest in the TTR locus, or replacement of a nucleic acid sequence of interest in the TTR locus (i.e., deleting a segment of the endogenous TTR locus and replacing with an orthologous human TTR sequence).

The exogenous repair templates can be for non-homologous-end-joining-mediated insertion or homologous recombination. Exogenous repair templates can comprise deoxyribonucleic acid (DNA) or ribonucleic acid (RNA), they can be single-stranded or double-stranded, and they can be in linear or circular form. For example, a repair template can be a single-stranded oligodeoxynucleotide (ssODN). Exogenous repair templates can also comprise a heterologous sequence that is not present at an untargeted endogenous TTR locus. For example, an exogenous repair template can comprise a selection cassette, such as a selection cassette flanked by recombinase recognition sites.

In cells other than one-cell stage embryos, the exogenous repair template can be a “large targeting vector” or “LTVEC,” which includes targeting vectors that comprise homology arms that correspond to and are derived from nucleic acid sequences larger than those typically used by other approaches intended to perform homologous recombination in cells. See, e.g., US 2004/0018626; WO 2013/163394; U.S. Pat. Nos. 9,834,786; 10,301,646; WO 2015/088643; U.S. Pat. Nos. 9,228,208; 9,546,384; 10,208,317; and US 2019-0112619, each of which is herein incorporated by reference in its entirety for all purposes. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

The methods can further comprise identifying a cell or animal having a modified target genomic locus. Various methods can be used to identify cells and animals having a targeted genetic modification. The screening step can comprise, for example, a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

The various methods provided herein allow for the generation of a genetically modified non-human F0 animal wherein the cells of the genetically modified F0 animal comprise the humanized TTR locus comprising the V30M mutation. It is recognized that depending on the method used to generate the F0 animal, the number of cells within the F0 animal that have the humanized TTR locus comprising the V30M mutation will vary. With mice, for example, the introduction of the donor ES cells into a pre-morula stage embryo from the mouse (e.g., an 8-cell stage mouse embryo) via, for example, the VELOCIMOUSE® method allows for a greater percentage of the cell population of the F0 mouse to comprise cells having the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification. The cells of the genetically modified F0 animal can be heterozygous for the humanized TTR locus comprising the V30M mutation or can be homozygous for the humanized TTR locus comprising the V30M mutation.

Likewise, various methods are provided for making a non-human animal comprising a synergistic activation mediator (SAM) expression cassette (comprising a chimeric Cas protein coding sequence and a chimeric adaptor protein expression coding sequence) and a humanized TTR locus comprising a V30M mutation as disclosed elsewhere herein. Any convenient method or protocol for producing a genetically modified organism is suitable for producing such a genetically modified non-human animal. See, e.g., Poueymirou et al. (2007) Nat. Biotechnol. 25(1):91-99; U.S. Pat. Nos. 7,294,754; 7,576,259; 7,659,442; 8,816,150; 9,414,575; 9,730,434; and 10,039,269, each of which is herein incorporated by reference in its entirety for all purposes (describing mouse ES cells and the VELOCIMOUSE® method for making a genetically modified mouse). See also US 2014/0235933 A1, US 2014/0310828 A1, each of which is herein incorporated by reference in its entirety for all purposes (describing rat ES cells and methods for making a genetically modified rat). See also Cho et al. (2009) Curr. Protoc. Cell. Biol. 42:19.11.1-19.11.22 (doi: 10.1002/0471143030.cb1911s42) and Gama Sosa et al. (2010) Brain Struct. Funct. 214(2-3):91-109, each of which is herein incorporated by reference in its entirety for all purposes. Such genetically modified non-human animals can be generated, for example, by creating a first non-human animal comprising a humanized TTR locus comprising a V30M mutation, creating a second non-human animal comprising a SAM expression cassette (e.g., genomically integrated SAM expression cassette), and then crossing the first and second non-human animals. Alternatively, such genetically modified non-human animals can be generated by making a genetically modified pluripotent cell comprising the humanized TTR locus comprising a V30M mutation, further modifying the pluripotent cell to comprise a SAM expression cassette (e.g., genomically integrated SAM expression cassette), and then generating a genetically modified non-human animal from the pluripotent cell. Likewise, such genetically modified non-human animals can be generated by making a genetically modified pluripotent cell comprising a SAM expression cassette (e.g., genomically integrated expression cassette), further modifying the pluripotent cell to comprise the humanized TTR locus comprising a V30M mutation, and then generating a genetically modified non-human animal from the pluripotent cell. Optionally, the cells are non-human animals can be further modified to comprise a guide RNA expression cassette and/or a recombinase expression cassette as described elsewhere herein.

For example, the method of producing a non-human animal comprising a humanized TTR locus comprising a V30M mutation and a SAM expression cassette (and optionally a guide RNA array expression cassette) can comprise: (1) providing a pluripotent cell (e.g., an embryonic stem (ES) cell such as a mouse ES cell or a rat ES cell) comprising in its genome the humanized TTR locus comprising a V30M mutation and the SAM expression cassette (and optionally the guide RNA array expression cassette); (2) introducing the genetically modified pluripotent cell into a non-human animal host embryo; and (3) gestating (e.g., implanting and gestating) the host embryo in a surrogate mother.

For example, the method of producing a non-human animal comprising a humanized TTR locus can comprise: (1) modifying the genome of a pluripotent cell (e.g., an embryonic stem (ES) cell such as a mouse ES cell or a rat ES cell) to comprise the humanized TTR locus comprising a V30M mutation; (2) identifying or selecting the genetically modified pluripotent cell comprising the humanized TTR locus comprising a V30M mutation; (3) introducing the genetically modified pluripotent cell into a non-human animal host embryo; and (4) gestating (e.g., implanting and gestating) the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized TTR locus comprising a V30M mutation.

Likewise, the method of producing a non-human animal comprising a SAM expression cassette and/or a guide RNA array expression cassette can comprise: (1) modifying the genome of a pluripotent cell to comprise one or more or all of the expression cassettes; (2) identifying or selecting the genetically modified pluripotent cell comprising the one or more or all of the expression cassettes; (3) introducing the genetically modified pluripotent cell into a non-human animal host embryo; and (4) gestating (e.g., implanting or gestating) the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising one or more or all of the expression cassettes. The non-human animal comprising the humanized TTR locus comprising a V30M mutation can then be crossed to the non-human animal comprising the SAM expression cassette and/or the guide RNA array expression cassette.

As another example, the method of producing a non-human animal comprising a humanized TTR locus comprising a V30M mutation and a SAM expression cassette can comprise: (1) modifying the genome of a pluripotent cell (e.g., an embryonic stem (ES) cell such as a mouse ES cell or a rat ES cell) to comprise the humanized TTR locus comprising a V30M mutation; (2) identifying or selecting the genetically modified pluripotent cell comprising the humanized TTR locus comprising a V30M mutation; (3) modifying the genome of the genetically modified pluripotent cell comprising the humanized TTR locus comprising a V30M mutation to comprise the SAM expression cassette; (4) identifying or selecting the genetically modified pluripotent cell comprising SAM expression cassette and the humanized TTR locus comprising a V30M mutation; (5) introducing the genetically modified pluripotent cell into a non-human animal host embryo; and (6) gestating (e.g., implanting and gestating) the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized TTR locus comprising a V30M mutation and the SAM expression cassette.

As another example, the method of producing a non-human animal comprising a humanized TTR locus comprising a V30M mutation and a SAM expression cassette can comprise: (1) modifying the genome of a pluripotent cell to comprise the SAM expression cassette; (2) identifying or selecting the genetically modified pluripotent cell comprising the SAM expression cassette; (3) modifying the genome of the genetically modified pluripotent cell comprising the SAM expression cassette to further comprise the humanized TTR locus comprising a V30M mutation; (4) identifying or selecting the genetically modified pluripotent cell comprising the humanized TTR locus comprising a V30M mutation and the SAM expression cassette; (5) introducing the genetically modified pluripotent cell into a non-human animal host embryo; and (6) gestating (e.g., implanting and gestating) the host embryo in a surrogate mother. The donor cell can be introduced into a host embryo at any stage, such as the blastocyst stage or the pre-morula stage (i.e., the 4-cell stage or the 8-cell stage). Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized TTR locus comprising a V30M mutation and the SAM expression cassette.

The methods can further comprise identifying a cell or animal having a modified target genomic locus (i.e., a humanized TTR locus comprising a V30M mutation and/or a target genomic locus comprising the SAM expression cassette or the guide RNA expression cassette). Various methods can be used to identify cells and animals having a targeted genetic modification.

The screening step can comprise, for example, a quantitative assay for assessing modification of allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). A modified pluripotent cell comprising a humanized TTR locus comprising a V30M mutation can be generated, for example, through recombination by (a) introducing into the cell one or more targeting vectors or exogenous donor nucleic acids comprising an insert nucleic acid flanked by 5′ and 3′ homology arms corresponding to 5′ and 3′ target sites, wherein the insert nucleic acid comprises a human TTR sequence comprising a V30M mutation; and (b) identifying at least one cell comprising in its genome the insert nucleic acid integrated at the endogenous Ttr locus comprising a V30M mutation. Alternatively, the modified pluripotent cell can be generated by (a) introducing into the cell: (i) a nuclease agent, wherein the nuclease agent induces a nick or double-strand break at a target sequence within the endogenous Ttr locus; and (ii) one or more targeting vectors comprising an insert nucleic acid flanked by 5′ and 3′ homology arms corresponding to 5′ and 3′ target sites located in sufficient proximity to the target sequence, wherein the insert nucleic acid comprises a human TTR sequence comprising a V30M mutation; and (c) identifying at least one cell comprising a modification (e.g., integration of the insert nucleic acid) at the endogenous Ttr locus. Any nuclease agent that induces a nick or double-strand break into a desired target sequence can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems or components of such systems (e.g., CRISPR/Cas9). See, e.g., US 2013/0309670 and US 2015/0159175, each of which is herein incorporated by reference in its entirety for all purposes.

Likewise, a modified pluripotent cell comprising a SAM expression cassette and/or a guide RNA expression cassette can be generated, for example, through recombination by (a) introducing into the cell one or more targeting vectors or exogenous donor nucleic acids comprising an insert nucleic acid flanked by 5′ and 3′ homology arms corresponding to 5′ and 3′ target sites, wherein the insert nucleic acid comprises the expression cassette; and (b) identifying at least one cell comprising in its genome the insert nucleic acid integrated at the target genomic locus. Alternatively, the modified pluripotent cell can be generated by (a) introducing into the cell: (i) a nuclease agent, wherein the nuclease agent induces a nick or double-strand break at a target sequence within the target genomic locus; and (ii) one or more targeting vectors comprising an insert nucleic acid flanked by 5′ and 3′ homology arms corresponding to 5′ and 3′ target sites located in sufficient proximity to the target sequence, wherein the insert nucleic acid comprises the expression cassette; and (c) identifying at least one cell comprising a modification (e.g., integration of the insert nucleic acid) at the target genomic locus. Any nuclease agent that induces a nick or double-strand break into a desired target sequence can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems or components of such systems (e.g., CRISPR/Cas9). See, e.g., US 2013/0309670 and US 2015/0159175, each of which is herein incorporated by reference in its entirety for all purposes.

In some such methods using cells other than one-cell stage embryos, the targeting vector is a large targeting vector at least 10 kb in length or in which the sum total of the 5′ and 3′ homology arms is at least 10 kb in length, but other types of exogenous donor nucleic acids can also be used and are well-known. See, e.g., US 2004/0018626; WO 2013/163394; U.S. Pat. Nos. 9,834,786; 10,301,646; WO 2015/088643; U.S. Pat. Nos. 9,228,208; 9,546,384; 10,208,317; and US 2019-0112619, each of which is herein incorporated by reference in its entirety for all purposes. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes. The 5′ and 3′ homology arms can correspond with 5′ and 3′ target sequences, respectively, that flank the region being replaced by the insert nucleic acid or that flank the region into which the insert nucleic acid is to be inserted. The exogenous donor nucleic acid or targeting vector can recombine with the target locus via homology directed repair or can be inserted via NHEJ-mediated insertion to generate the modified genomic locus.

The donor cell can be introduced into a host embryo at any stage, such as the blastocyst stage or the pre-morula stage (i.e., the 4-cell stage or the 8-cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo (e.g., that already comprises a SAM expression cassette) to comprise the humanized TTR locus comprising a V30M mutation using the methods described above for modifying pluripotent cells; (2) selecting the genetically modified embryo; and (3) gestating (e.g., implanting and gestating) the genetically modified embryo into a surrogate mother. Progeny that are capable of transmitting the genetic modification though the germline are generated.

Alternatively, the method of producing the non-human animals described elsewhere herein can comprise: (1) modifying the genome of a one-cell stage embryo (e.g., that already comprises a humanized TTR locus comprising a V30M mutation) to comprise a SAM expression cassette (and optionally a guide RNA expression cassette) using the methods described above for modifying pluripotent cells; (2) selecting the genetically modified embryo; and (3) gestating (e.g., implanting and gestating) the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the humanized TTR locus comprising a V30M mutation and/or the SAM expression cassette. Depending on the method used to generate the F0 animal, the number of cells within the F0 animal that have the humanized TTR locus comprising a V30M mutation and/or the SAM expression cassette will vary. With mice, for example, the introduction of the donor ES cells into a pre-morula stage embryo from the mouse (e.g., an 8-cell stage mouse embryo) via, for example, the VELOCIMOUSE® method allows for a greater percentage of the cell population of the F0 mouse to comprise cells having the targeted genetic modification. For example, at least 50%, at least 60%, at least 65%, at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% of the cellular contribution of the non-human F0 animal can comprise a cell population having the targeted modification.

The cells of the genetically modified F0 animal can be heterozygous for the humanized TTR locus comprising a V30M mutation and/or the SAM expression cassette or the guide RNA expression cassette or can be homozygous for the humanized TTR locus comprising a V30M mutation and/or the SAM expression cassette or the guide RNA expression cassette.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ ID NO | Type | Description |
|---|---|---|
| 1 | Protein | Human TTR Protein NP_000362.1 and P02766.1 |
| 2 | Protein | Human TTR V30M |
| 3 | Protein | Human TTR Signal Peptide |
| 4 | Protein | Human TTR Mature Protein (no Signal Peptide) |
| 5 | Protein | Human TTR V30M Mature Protein (no Signal Peptide) |
| 6 | DNA | Human TTR CDS |
| 7 | DNA | Human TTR V30M CDS |
| 8 | DNA | Human TTR Signal Peptide CDS |
| 9 | DNA | Human TTR Mature Protein (no Signal Peptide) CDS |
| 10 | DNA | Human TTR V30M Mature Protein (no Signal Peptide) CDS |
| 11 | DNA | Human TTR cDNA NM_000371.3 |
| 12 | DNA | Human TTR Gene NG_009490.1 |
| 13 | Protein | Mouse TTR Protein P07309.1 and NP_038725.1 |
| 14 | Protein | Mouse TTR Signal Peptide |
| 15 | Protein | Mouse TTR Mature Protein (no Signal Peptide) |
| 16 | DNA | Mouse Ttr CDS |
| 17 | DNA | Mouse Ttr Signal Peptide CDS |
| 18 | DNA | Mouse Ttr Mature Protein (no Signal Peptide) CDS |
| 19 | DNA | Mouse Ttr cDNA NM_013697.5 |
| 20 | DNA | Mouse Ttr gene NC_000084.6 |
| 21 | DNA | Mouse Ttr Sequence (Start Codon to Stop Codon) |
| 22 | DNA | V30M Humanization - F0, with Cassette |
| 23 | DNA | V30M Humanization - F1, Cassette Deleted |
| 24 | DNA | Human TTR Sequence at Humanized Locus |
| 25-96 | DNA | Primers and Probes |
| 97 | Protein | dCas9-VP64 chimeric Cas protein |
| 98 | Protein | dCas9 protein |
| 99 | Protein | VP64 transcriptional activation domain |
| 100 | Protein | Linker v1 |
| 101 | Protein | Linker v2 |
| 102 | Protein | MCP-p65-HSF1 chimeric adaptor protein |
| 103 | Protein | MS2 coat protein (MCP) |
| 104 | Protein | p65 transcriptional activation domain |
| 105 | Protein | HSF1 transcriptional activation domain |
| 106 | RNA | MS2-binding loop |
| 107 | Protein | T2A |
| 108 | Protein | P2A |
| 109 | Protein | E2A |
| 110 | Protein | F2A |
| 111 | DNA | Nucleic acid encoding dCas9 protein |
| 112 | DNA | Nucleic acid encoding dCas9-VP64 chimeric Cas protein |
| 113 | DNA | Nucleic acid encoding MCP |
| 114 | DNA | Nucleic acid encoding MCP-p65-HSF1 chimeric adaptor protein |
| 115 | DNA | Nucleic acid encoding VP64 transcriptional activation domain |
| 116 | DNA | Nucleic acid encoding p65 transcriptional activation domain |
| 117 | DNA | Nucleic acid encoding HSF1 transcriptional activation domain |
| 118 | DNA | Synergistic activation mediator (SAM) bicistronic expression cassette |
| | | (dCas9-VP64-T2A-MCP-p65-HSF1) |
| 119 | DNA | Generic guide RNA array expression cassette |
| 120 | DNA | Ttr guide RNA array expression cassette |
| 121 | DNA | Mouse Ttr guide RNA target sequence v1 |
| 122 | DNA | Mouse Ttr guide RNA target sequence v2 |
| 123 | DNA | Mouse Ttr guide RNA target sequence v3 |
| 124 | RNA | Mouse Ttr single guide RNA v1 |
| 125 | RNA | Mouse Ttr single guide RNA v2 |
| 126 | RNA | Mouse Ttr single guide RNA v3 |
| 127 | RNA | gRNA scaffold with MS2 binding loops |
| 128 | RNA | Mouse Ttr guide RNA DNA-targeting segment v1 |
| 129 | RNA | Mouse Ttr guide RNA DNA-targeting segment v2 |
| 130 | RNA | Mouse Ttr guide RNA DNA-targeting segment v3 |
| 131 | Protein | Synergistic activation mediator (SAM) |
| | | (dCas9-VP64-T2A-MCP-p65-HSF1) |
| 132 | RNA | Generic single gRNA with MS2 binding loops |
| 133 | DNA | Synergistic activation mediator (SAM) coding sequence |
| | | (dCas9-VP64-T2A-MCP-p65-HSF1) |
| 134 | DNA | Generic guide RNA array promoters and guide RNA coding sequences |
| 135 | DNA | Ttr guide RNA array promoters and guide RNA coding sequences |
| 136 | DNA | pscAAV Ttr array |
| 137 | DNA | pAAV Ttr g1 |
| 138 | DNA | pAAV Ttr g2 |
| 139 | DNA | pAAV Ttr g3 |
| 140 | RNA | gRNA scaffold with MS2 binding loops v2 |
| 141 | RNA | Generic single gRNA with MS2 binding loops v2 |
| 142 | RNA | crRNA Tail |
| 143 | RNA | TracrRNA v1 |
| 144 | RNA | TracrRNA v2 |
| 145 | RNA | TracrRNA v3 |
| 146 | RNA | Guide RNA Scaffold v1 |
| 147 | RNA | Guide RNA Scaffold v2 |
| 148 | RNA | Guide RNA Scaffold v3 |
| 149 | RNA | Guide RNA Scaffold v4 |
| 150 | RNA | Guide RNA Scaffold v5 |
| 151 | RNA | Guide RNA Scaffold v6 |
| 152 | RNA | Guide RNA Scaffold v7 |
| 153 | DNA | Guide RNA Target Sequence Plus PAM v1 |
| 154 | DNA | Guide RNA Target Sequence Plus PAM v2 |
| 155 | DNA | Guide RNA Target Sequence Plus PAM v3 |

### EXAMPLES

### Example 1

### Generation of Mice Comprising a Humanized TTR Locus with a V30M Mutation

A humanized TTR allele was generated that was a complete deletion of the mouse transthyretin coding sequence and its replacement with the orthologous part of the human TTR gene. The orthologous part of the human TTR gene encoded a V30M point mutation. The nomenclature of the amino acid position for the V30M mutation refers to the mature TTR protein after cleavage of the 20 amino acid signal peptide. This nomenclature is consistent with nomenclature used in publications describing this mutation.

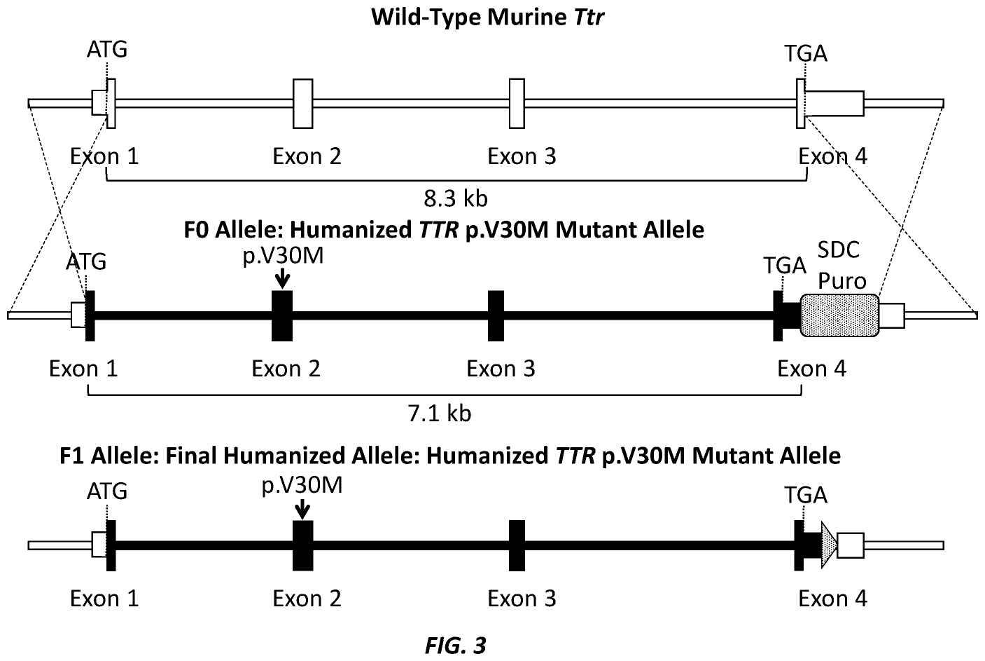

A large targeting vector comprising a 5′ homology arm including 33.7 kb of sequence upstream from the mouse Ttr start codon and 34.5 kb of the sequence downstream of the mouse Ttr stop codon was generated to replace the approximately 8.3 kb region from the mouse Ttr start codon to the mouse Ttr stop codon with the approximately 7.1 kb orthologous human TTR sequence from the human TTR start codon to the end of the last human TTR exon (exon 4, including the human 3′ UTR) and a self-deleting puromycin selection cassette (SDC Puro) flanked by loxP sites. See FIG. 3. The SDC Puro cassette included the following components from 5′ to 3′: loxP site; mouse protamine (Prm1) promoter; Crei (Cre coding sequence optimized to include intron); polyA; human ubiquitin promoter; puromycin-N-acetyltransferase (puroᵣ) coding sequence; polyA; and loxP site. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

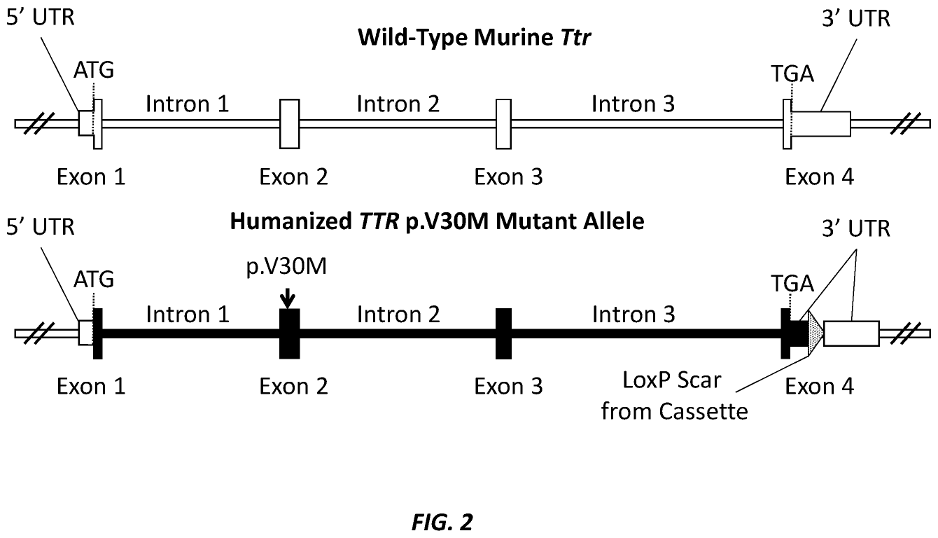

The allele with the loxP-mPrml-Crei-pA-hUbl-em7-Neo-pA-loxP cassette is set forth in SEQ ID NO: 22. See FIG. 3. After cassette deletion, loxP and cloning sites remained downstream of human 3′ UTR. The cassette-deleted allele is set forth in SEQ ID NO: 23. See FIGS. 2 and 3.

Sequences for the mouse TTR signal peptide and mature protein (i.e., after cleavage of the 20 amino acid signal peptide) are set forth in SEQ ID NOS: 14 and 15, respectively, with the corresponding coding sequence set forth in SEQ ID NOS: 17 and 18, respectively. Sequences for the wild type human TTR signal peptide and mature protein are set forth in SEQ ID NOS: 3 and 4, respectively, with the corresponding coding sequences set forth in SEQ ID NOS: 8 and 9, respectively. The sequence for the V30M version of the human TTR mature protein is set forth in SEQ ID NO: 5, with the corresponding coding sequence set forth in SEQ ID NO: 10. An alignment of the mouse and human TTR proteins is shown in FIG. 1. The mouse and human wild type TTR coding sequences are set forth in SEQ ID NOS: 16 and 6, respectively. The human V30M TTR coding sequence is set forth in SEQ ID NO: 7. The mouse and human wild type TTR protein sequences are set forth in SEQ ID NOS: 13 and 1, respectively. The human V30M TTR protein sequence is set forth in SEQ ID NO: 2. The sequences for the expected humanized V30M TTR coding sequence and the expected humanized V30M TTR protein are set forth in SEQ ID NOS: 7 and 2, respectively.

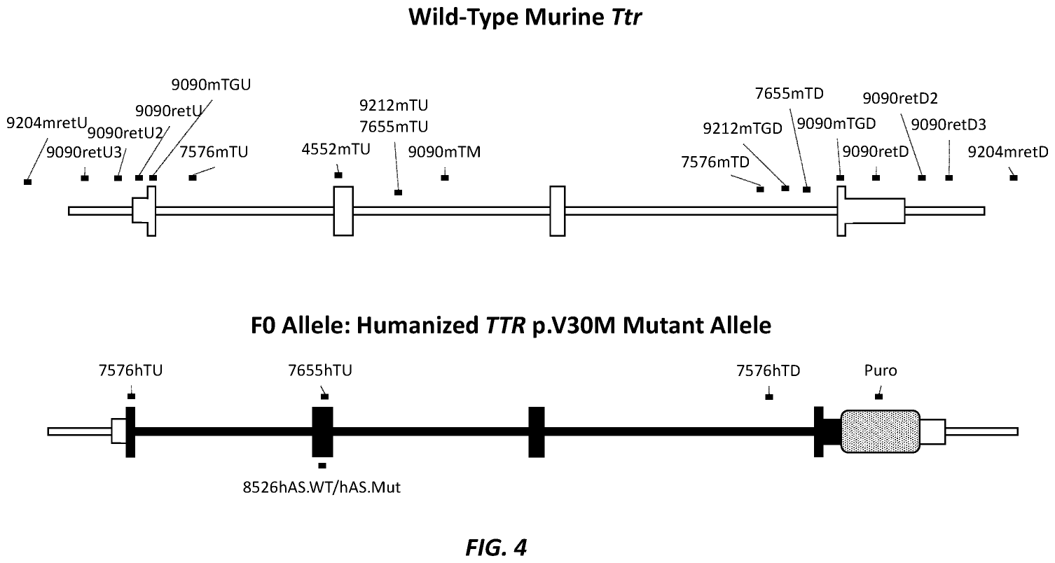

To generate the mutant allele, the large targeting vector described above was introduced into F1H4 mouse embryonic stem (ES) cells together with CRISPR/Cas9 components targeting the mouse Ttr locus. F1H4 mouse ES cells were derived from hybrid embryos produced by crossing a female C57BL/6NTac mouse to a male 129S6/SvEvTac mouse. See, e.g., US 2015-0376651 and WO 2015/200805, each of which is herein incorporated by reference in its entirety for all purposes. Following antibiotic selection, colonies were picked, expanded, and screened by TAQMAN®. See FIG. 4. Loss-of-allele assays were performed to detect loss of the endogenous mouse allele, and gain-of-allele assays were performed to detect gain of the humanized allele using the primers and probes set forth in Table 3. Retention assays and CRISPR assays using primers and probes were also performed using the primers and probes set forth in FIG. 4 and in Table 3.

TAB;E 3
Screening Assays.

| Assay | | | |
|---|---|---|---|
| Name | Forward Primer | Reverse Primer | Probe |
| 9090 | CACAGACAATCAGACGTACC | GGGACATCTCGGTTTCCTGACTT | TCATGTAATCTGGCTTCAGAGTGGGA |
| retU3 | AGTA (SEQ ID NO: 25) | (SEQ ID NO: 26) | (SEQ ID NO: 27) |
| 9090 | CCAGCTTTGCCAGTTTACGA | TCCACACTACTGAACTCCACAA | TGGGAGGCAATTCTTAGTTTCAATGGA |
| retU2 | (SEQ ID NO: 28) | (SEQ ID NO: 29) | (SEQ ID NO: 30) |
| 9090 | TTGGACGGTTGCCCTCTT | CGGAACACTCGCTCTACGAAA | TCCCAAAGGTGTCTGTCTGCACA |
| retU | (SEQ ID NO: 31) | (SEQ ID NO: 32) | (SEQ ID NO: 33) |
| 9090 | GATGGCTTCCCTTCGACTCTT | GGGCCAGCTTCAGACACA | CTCCTTTGCCTCGCTGGACTGG |
| mTGU | C | (SEQ ID NO: 35) | (SEQ ID NO: 36) |
| | (SEQ ID NO: 34) | | |
| 7576 | CACTGACATTTCTCTTGTCTC | CCCAGGGTGCTGGAGAATCCAA | CGGACAGCATCCAGGACTT |
| mTU | CTCT (SEQ ID NO: 37) | (SEQ ID NO: 38) | (SEQ ID NO: 39) |
| 9090 | GGGCTCACCACAGATGAGAA | GCCAAGTGTCTTCCAGTACGAT | AGAAGGAGTGTACAGAGTAGAACTGG |
| mTM | G | (SEQ ID NO: 41) | ACA |
| | (SEQ ID NO: 40) | | (SEQ ID NO: 42) |
| 7576 | CACTGTTCGCCACAGGTCTT | GTTCCCTTTCTTGGGTTCAGA | TGTTTGTGGGTGTCAGTGTTTCTACTC |
| mTD | (SEQ ID NO: 43) | (SEQ ID NO: 44) | (SEQ ID NO: 45) |
| 9090 | GCTCAGCCCATACTCCTACA | GATGCTACTGCTTTGGCAAGATC | CACCACGGCTGTCGTCAGCAA |
| mTGD | (SEQ ID NO: 46) | (SEQ ID NO: 47) | (SEQ ID NO: 48) |
| 9090 | GCCCAGGAGGACCAGGAT | CCTGAGCTGCTAACACGGTT | CTTGCCAAAGCAGTAGCATCCCA |
| retD | (SEQ ID NO: 49) | (SEQ ID NO: 50) | (SEQ ID NO: 51) |
| 9090 | GGCAACTTGCTTGAGGAAGA | AGCTACAGACCATGCTTAGTGTA | AGGTCAGAAAGCAGAGTGGACCA |
| retD2 | (SEQ ID NO: 52) | (SEQ ID NO: 53) | (SEQ ID NO: 54) |
| 9090 | GCAGCAACCCAGCTTCACTT | TGCCAGTTTAGGAGGAATATGTT | CCCAGGCAATTCCTACCTTCCCA |
| retD3 | (SEQ ID NO: 55) | C | (SEQ ID NO: 57) |
| | | (SEQ ID NO: 56) | |
| 7576 | ACTGAGCTGGGACTTGAAC | CTGAGGAAACAGAGGTACCAGA | TCTGAGCATTCTACCTCATTGCTTTGG |
| hTU | (SEQ ID NO: 58) | TAT | T |
| | | (SEQ ID NO: 59) | (SEQ ID NO: 60) |
| 7576 | TGCCTCACTCTGAGAACCA | AGTCACACAGTTCTGTCAAATCA | AGGCTGTCCCAGCACCTGAGTCG |
| hTD | (SEQ ID NO: 61) | G | (SEQ ID NO: 63) |
| | | (SEQ ID NO: 62) | |
| Puro | CGCAACCTCCCCTTCTACG | GTCCTTCGGGCACCTCG | CGGCTCGGCTTCACCGTCACC |
| | (SEQ ID NO: 64) | (SEQ ID NO: 65) | (SEQ ID NO: 66) |
| 7655 | GGCCGTGCATGTGTTCAG | TCCTGTGGGAGGGTTCTTTG | AAGGCTGCTGATGACACCTGGGA |
| hTU | (SEQ ID NO: 67) | (SEQ ID NO: 68) | (SEQ ID NO: 69) |
| 9212 | GGTTCCCATTTGCTCTTATTC | CCCTCTCTCTGAGCCCTCTA | AGATTCAGACACACACAACTTACCAG |
| mTU | GT | (SEQ ID NO: 71) | C |
| | (SEQ ID NO: 70) | | (SEQ ID NO: 72) |
| 9212 | CCCACACTGCAGAAGGAAAC | GCTGCCTAAGTCTTTGGAGCT | AGACCTGCAATTCTCTAAGAGCTCCAC |
| mTGD | TTG | (SEQ ID NO: 74) | A |
| | (SEQ ID NO: 73) | | (SEQ ID NO: 75) |
| 7655 | GGTTCCCATTTGCTCTTATTC | CCCTCTCTCTGAGCCCTCTA | AGATTCAGACACACACAACTTACCAG |
| mTU | GT | (SEQ ID NO: 77) | C |
| | (SEQ ID NO: 76) | | (SEQ ID NO: 78) |
| 7655 | CCAGCTTAGCATCCTGTGAA | GAGAGGAGAGACAGCTAGTTCT | TTGTCTGCAGCTCCTACCTCTGGG |
| mTD | CA | AAC | (SEQ ID NO: 81) |
| | (SEQ ID NO: 79) | (SEQ ID NO: 80) | |
| 9204 | GGCAACTTGCTTGAGGAAGA | AGCTACAGACCATGCTTAGTGTA | AGGTCAGAAAGCAGAGTGGACCA |
| mretD | (SEQ ID NO: 82) | (SEQ ID NO: 83) | (SEQ ID NO: 84) |
| 9204 | TGTGGAGTTCAGTAGTGTGG | GCCCTCTTCATACAGGAATCAC | TTGACATGTGTGGGTGAGAGATTTTAC |
| mretU | AG | (SEQ ID NO: 86) | TG |
| | (SEQ ID NO: 85) | | (SEQ ID NO: 87) |
| 4552 | CACTGACATTTCTCTTGTCTC | CGGACAGCATCCAGGACTT | CCCAGGGTGCTGGAGAATCCAA |
| mTU | CTCT (SEQ ID NO: 88) | (SEQ ID NO: 89) | (SEQ ID NO: 90) |
| 8526hA | CTGTCCGAGGCAGTCCTG | GTGTCATCAGCAGCCTTTCTG | AATGTGGCCGTGCATG |
| S.WT | (SEQ ID NO: 91) | (SEQ ID NO: 92) | (SEQ ID NO: 93) |
| 8526hA | CTGTCCGAGGCAGTCCTG | GTGTCATCAGCAGCCTTTCTG | ATGTGGCCATGCATG |
| S.MUT | (SEQ ID NO: 94) | (SEQ ID NO: 95) | (SEQ ID NO: 96) |

Modification-of-allele (MOA) assays including loss-of-allele (LOA) and gain-of-allele (GOA) assays are described, for example, in US 2014/0178879; US 2016/0145646; WO 2016/081923; and Frendewey et al. (2010) Methods Enzymol. 476:295-307, each of which is herein incorporated by reference in its entirety for all purposes. The loss-of-allele (LOA) assay inverts the conventional screening logic and quantifies the number of copies in a genomic DNA sample of the native locus to which the mutation was directed. In a correctly targeted heterozygous cell clone, the LOA assay detects one of the two native alleles (for genes not on the X or Y chromosome), the other allele being disrupted by the targeted modification. The same principle can be applied in reverse as a gain-of-allele (GOA) assay to quantify the copy number of the inserted targeting vector in a genomic DNA sample.

Retention assays are described in US 2016/0145646 and WO 2016/081923, each of which is herein incorporated by reference in its entirety for all purposes. Retention assays distinguish between correct targeted insertions of a nucleic acid insert into a target genomic locus from random transgenic insertions of the nucleic acid insert into genomic locations outside of the target genomic locus by assessing copy numbers of DNA templates from 5′ and 3′ target sequences corresponding to the 5′ and 3′ homology arms of the targeting vector, respectively. Specifically, retention assays determine copy numbers in a genomic DNA sample of a 5′ target sequence DNA template intended to be retained in the modified target genomic locus and/or the 3′ target sequence DNA template intended to be retained in the modified target genomic locus. In diploid cells, correctly targeted clones will retain a copy number of two. Copy numbers greater than two generally indicate transgenic integration of the targeting vector randomly outside of the target genomic locus rather than at the target genomic locus. Copy numbers of less than generally indicate large deletions extending beyond the region targeted for deletion.

CRISPR assays are TAQMAN® assays designed to cover the region that is disrupted by the CRISPR gRNAs. When a CRISPR gRNA cuts and creates an indel (insertion or deletion), the TAQMAN® assay will fail to amplify and thus reports CRISPR cleavage.

The 8526hAS.WT and 8526hAS.MUT assays in combination detect non-mutated and mutated (V30M) alleles.

F0 mice were generated from the modified ES cells using the VELOCIMOUSE® method. Specifically, mouse ES cell clones comprising the humanized V30M TTR locus described above that were selected by the MOA assay described above were injected into 8-cell stage embryos using the VELOCIMOUSE® method. See, e.g., U.S. Pat. Nos. 7,576,259; 7,659,442; 7,294,754; US 2008/0078000; and Poueymirou et al. (2007) Nat. Biotechnol. 25(1):91-99, each of which is herein incorporated by reference in its entirety for all purposes. In the VELOCIMOUSE® method, targeted mouse ES cells are injected through laser-assisted injection into pre-morula stage embryos, e.g., eight-cell-stage embryos, which efficiently yields F0 generation mice that are fully ES-cell-derived. In the VELOCIMOUSE® method, the injected pre-morula stage embryos are cultured to the blastocyst stage, and the blastocyst-stage embryos are introduced into and gestated in surrogate mothers to produce the F0 generation mice. When starting with mouse ES cell clones homozygous for the targeted modification, F0 mice homozygous for the targeted modification are produced. When starting with mouse ES cell clones heterozygous for the targeted modification, subsequent breeding can be performed to produce mice homozygous for the targeted modification.

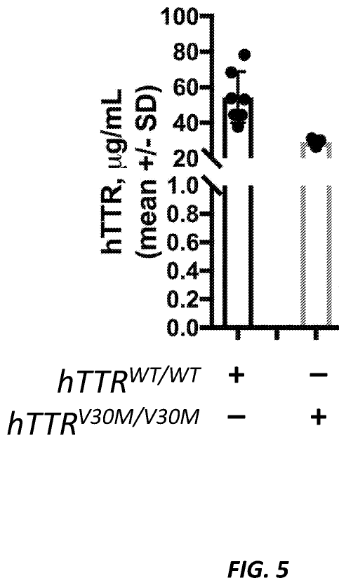

A human TTR ELISA kit (Aviva Systems Biology; Cat No.: OKIA00081; 1:2000 dilution) was then used to assess blood plasma human TTR levels in wild type humanized TTR mice and V30M humanized TTR mice. The data are summarized in FIG. 5. As shown in FIG. 5, the wild type humanized TTR mice had ˜55 μg/mL circulating hTTR. V30M humanized TTR mice had ˜30 μg/mL circulating hTTR.

### Example 2

### Seeding of Mice Comprising a Humanized TTR Locus with a V30M Mutation with Pre-Formed TTR Aggregates

The mice comprising a humanized TTR locus with a V30M mutation as described in Example 1 are seeded via peripheral injection of pre-formed TTR aggregates. In a first experiment, tail vein injection is used. To introduce TTR aggregates into general circulation, pre-formed V30M TTR fibrils (200 micrograms in a total volume of 100 microliters of PBS) are injected into the tail vein of 8-12 week old mice. Injection of the exogenous fibrils into systemic circulation facilitates seeding by endogenous circulating V30M TTR. In some samples, seeding is potentiated by co-injection with heparin (8 units/20 g body weight), which has been reported to accelerate TTR amyloid deposition by serving as a template for amyloid fibrils to form. See, e.g., Noborn et al. (2011) Proc. Natl. Acad. Sci. U.S.A. 108(14):5584-5589, herein incorporated by reference in its entirety for all purposes.

In a second experiment, hydrodynamic delivery is used to deliver pre-formed TTR aggregates. In this approach, 200 micrograms of V30M TTR aggregates in a total volume of ˜1 mL of lactated Ringer's solution are rapidly injected in the tail vein of mice. Rapid delivery of this large volume induces pore formation in liver fenestrae and in hepatocytes, allowing the aggregates to enter hepatocytes. Other sites in the body that receive the hydrodynamic delivery cargo (although to a lesser extent than the liver) include the lung, heart, spleen, kidney. Many of these organs are sites of exogenous TTR deposition. The delivery of TTR aggregates accelerates seeding and templating of endogenous TTR on the exogenously introduced aggregates.

In each experiment, TTR amyloid formation is longitudinally monitored after seeding in the mice using submandibular (or retro-orbital) bleeds. Mice are monitored for behavioral and autonomic function, including sweat testing, pupillary reflex response, grip strength, and latency to respond to cold and/or hot stimuli.

### Example 3

### Generation and Validation of SAM Mice

Mice comprising genomically integrated dCas9 synergistic activation mediator (SAM) system components (dCas9-VP64 and MCP-p65-HSF1) as one transcript driven by the endogenous Rosa26 promoter were generated as described in US 2019/0284572 and WO 2019/183123, each of which is herein incorporated by reference in its entirety for all purposes. Initially, expression of the dCas9 SAM system is blocked by the presence of a foxed neomycin stop cassette. Upon introduction of Cre recombinase, the stop cassette is deleted and dCas9 SAM expression is turned on. We can then introduce guide RNAs or guide RNA arrays (e.g., expressed from a U6 promoter) by integrating them into the other Rosa26 allele for constitutive activation or LNP/AAV introduction for more transient activations. By pairing the dCas9 SAM allele with various Cre delivery methods, we can control the timing and tissue specificity of gene modulation.

The S. pyogenes dCas9 coding sequence (CDS) in the expression cassette was codon-optimized for expression in mice. The encoded dCas9 includes the following mutations to render the Cas9 nuclease-inactive: D10A and N863A. The NLS-dCas9-NLS-VP64-T2A-MCP-NLS-p65-HSF1 expression cassette is depicted in FIG. 6A and SEQ ID NO: 118. The synergistic activation mediator (SAM) coding sequence (dCas9-VP64-T2A-MCP-p65-HSF1 or more specifically NLS-dCas9-NLS-VP64-T2A-MCP-NLS-p65-HSF1) is set forth in SEQ ID NO: 133 and encodes the protein set forth in SEQ ID NO: 131. The expression cassette was targeted to the first intron of the Rosa26 locus (see FIG. 7) to take advantage of the strong universal expression of the Rosa26 locus and the ease of targeting the Rosa26 locus. The expression cassette was preceded by a foxed neomycin resistance cassette (neo cassette) with appropriate splicing signals and a strong polyadenylation (polyA) signal. The components of the dCas9 SAM expression cassette from 5′ to 3′ are shown in Table 4 below.

TABLE 4
dCas9 SAM Expression Cassette Components.

| | Nucleotide Region Within |
|---|---|
| Component | SEQ ID NO: 118 |
| First loxP site | 1-34 |
| Sequence encoding neomycin phosphotransferase for resistance to | 125-928 |
| neomycin family antibiotics (e.g. G418) | |
| Polyadenylation signal | 937-2190 |
| Second loxP site | 2218-2251 |
| Codon-optimized dCas9 coding sequence | 2306-6457 |
| NLS | 2309-2356 |
| NLS | 6512-6532 |
| VP64 | 6533-6719 |
| T2A with 5’ GSG | 6719-6781 |
| MCP | 6782-7171 |
| NLS | 7226-7246 |
| p65 | 7262-7804 |
| HSF1 | 7829-8200 |
| Woodchuck hepatitis virus posttranscriptional regulatory | 8224-8820 |
| element (WPRE) | |

Prior to removal of the floxed neomycin resistance cassette (neo cassette) by the action of Cre recombinase, the neomycin resistance gene is transcribed and translated; however, the dCas9-NLS-VP64 CDS and MCP-NLS-p65-HSF1 CDS are not expressed due to the presence of the strong poly(A) region, which effectively blocks run-through transcription. See FIG. 6A. Upon removal of the neo cassette by the action of Cre recombinase, however, the hybrid mRNA for the dCas9 and MCP fusion proteins is constitutively expressed by the Rosa26 promoter. See FIG. 6B. dCas9 and MCP expression were validated as described in US 2019/0284572 and WO 2019/183123, each of which is herein incorporated by reference in its entirety for all purposes. The system was validated in vivo using the Ttr guide RNA array depicted in FIG. 8 and in SEQ ID NO: 120, as described in US 2019/0284572 and WO 2019/183123, each of which is herein incorporated by reference in its entirety for all purposes. The region including the promoters and guide RNA coding sequences is set forth in SEQ ID NO: 135. The guide RNA target sequences (not including PAM) in the mouse Ttr gene that are targeted by the guide RNAs in the array are set forth in SEQ ID NO: 121 (ACGGTTGCCCTCTTTCCCAA), SEQ ID NO: 122 (ACTGTCAGACTCAAAGGTGC), and SEQ ID NO: 123 (GACAATAAGTAGTCTTACTC), respectively. SEQ ID NO: 121 is located −63 of the Ttr transcription start site, SEQ ID NO: 122 is located −134 of the Ttr transcription start site, and SEQ ID NO: 123 is located −112 of the Ttr transcription start site. The single guide RNAs targeting these guide RNA target sequences are set forth in SEQ ID NOS: 124, 125, and 126, respectively. The guides were designed to direct the dCas9 SAM components to the 100-200 bp region upstream of the Ttr transcriptional start site (TSS). See FIG. 9. The components of the Ttr guide RNA array expression cassette from 5′ to 3′ are shown in Table 5 below. A general schematic of the structure of each guide RNA, including the MS2 stem loops, is shown in FIG. 10.

TABLE 5
Ttr Guide RNA Array Expression Cassette Components.

| | Nucleotide Region Within |
|---|---|
| Component | SEQ ID NO: 120 |
| First rox site | 1-32 |
| Sequence encoding puromycin-N-acetyltransferase for | 111-710 |
| resistance to puromycin family antibiotics | |
| Polyadenylation signal | 797-2338 |
| Second rox site | 2363-2394 |
| First U6 promoter | 2401-2640 |
| First Ttr guide RNA coding sequence | 2642-2798 |
| Second U6 promoter | 2884-3123 |
| Second Ttr guide RNA coding sequence | 3125-3281 |
| Third U6 promoter | 3366-3605 |
| Third Ttr guide RNA coding sequence | 3606-3762 |

### Example 4

### Generation of SAM Mice Comprising a Humanized TTR Locus with a V30M Mutation

As shown in Example 1, we have created humanized alleles that can be used to model the protein aggregation diseases associated with V30M TTR alleles. As shown in Example 3, using the SAM system, we first validated that we can precisely overexpress the murine Ttr gene in mouse embryonic stem cells (mESC) and in vivo.

Next, we sought to translate this increased expression to our humanized TTR V30M model. Mice comprising a heterozygous humanization of TTR with a V30M mutation and heterozygous SAM (Ttr^{huV30M/+}; R26^{SAM/+}) were generated through breeding. Circulating human TTR (hTTR) and mouse TTR (mTTR) are determined in mice prior to injection with AAV8^{GFP} or AAV8^{3aTtr} and at various time points post-injection. Animals are either non-injected or are tail vein injected with either AAV8^{GFP} or AAV8 carrying TTR guides (AAV8^{3aTtr}). Serum is collected prior to the day of tail vein injection, and is collected again at various time points post-injection.

Mice comprising homozygous humanization of TTR with a V30M mutation and homozygous SAM (Ttr^{huV30M/huV30M}; R26^{SAM/SAM}) are then generated through breeding. Circulating hTTR is determined in prior to injection with AAV8^{GFP} or AAV8^{3aTtr} and at various time points after injection. An increase of circulating hTTR V30M is observed in Ttr^{hu/hu}; R26^{SAM/SAM} mice injected with AAV8^{3aTtr} whereas control injections of AAV8^{GFP} do not have any impact on circulating TTR levels.

### Example 5

### Seeding of SAM Mice Comprising a Humanized TTR Locus with a V30M Mutation with Pre-Formed TTR Aggregates

The SAM mice comprising a humanized TTR locus with a V30M mutation as described in Example 4 are seeded via peripheral injection of pre-formed TTR aggregates. In a first experiment, tail vein injection is used. To introduce TTR aggregates into general circulation, pre-formed V30M TTR fibrils (200 micrograms in a total volume of 100 microliters of PBS) are injected into the tail vein of 8-12 week old mice. Injection of the exogenous fibrils into systemic circulation facilitates seeding by endogenous circulating V30M TTR. In some samples, seeding is potentiated by co-injection with heparin (8 units/20 g body weight), which has been reported to accelerate TTR amyloid deposition by serving as a template for amyloid fibrils to form. See, e.g., Noborn et al. (2011) Proc. Natl. Acad. Sci. U.S.A. 108(14):5584-5589, herein incorporated by reference in its entirety for all purposes.

In a second experiment, hydrodynamic delivery is used to deliver pre-formed TTR aggregates. In this approach, 200 micrograms of V30M TTR aggregates in a total volume of ˜1 mL of lactated Ringer's solution are rapidly injected in the tail vein of mice. Rapid delivery of this large volume induces pore formation in liver fenestrae and in hepatocytes, allowing the aggregates to enter hepatocytes. Other sites in the body that receive the hydrodynamic delivery cargo (although to a lesser extent than the liver) include the lung, heart, spleen, kidney. Many of these organs are sites of exogenous TTR deposition. The delivery of TTR aggregates accelerates seeding and templating of endogenous TTR on the exogenously introduced aggregates.

In each experiment, TTR amyloid formation is longitudinally monitored after seeding in the mice using submandibular (or retro-orbital) bleeds. Mice are monitored for behavioral and autonomic function, including sweat testing, pupillary reflex response, grip strength, and latency to respond to cold and/or hot stimuli.

We claim:

1. A non-human animal comprising in its genome a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous TTR locus comprises a V30M mutation.

2. The non-human animal of claim 1, wherein the human TTR sequence comprises the V30M mutation.

3. The non-human animal of claim 1 or 2, wherein the humanized endogenous TTR locus comprises an endogenous TTR promoter, wherein the human TTR sequence is operably linked to the endogenous TTR promoter.

4. The non-human animal of any preceding claim, wherein at least one intron and at least one exon of the endogenous TTR locus have been deleted and replaced with the corresponding human TTR sequence.

5. The non-human animal of any preceding claim, wherein the humanized endogenous TTR locus comprises a human TTR 3′ untranslated region.

6. The non-human animal of any preceding claim, wherein the humanized endogenous TTR locus comprises an endogenous TTR 3′ untranslated region.

7. The non-human animal of any preceding claim, wherein the humanized endogenous TTR locus comprises a human TTR 3′ untranslated region and an endogenous TTR 3′ untranslated region.

8. The non-human animal of any preceding claim, wherein the endogenous TTR 5′ untranslated region has not been deleted and replaced with the corresponding human TTR sequence.

9. The non-human animal of any preceding claim, wherein the humanized endogenous TTR locus encodes a transthyretin precursor protein comprising a human mature transthyretin protein sequence.

10. The non-human animal of claim 9, wherein the human mature transthyretin protein sequence comprises the sequence set forth in SEQ ID NO: 5, and optionally wherein the human mature transthyretin protein sequence is encoded by a sequence comprising the sequence set forth in SEQ ID NO: 10.

11. The non-human animal of any preceding claim, wherein the humanized endogenous TTR locus encodes a transthyretin precursor protein comprising a human transthyretin signal peptide sequence.

12. The non-human animal of claim 11, wherein the human transthyretin signal peptide sequence comprises the sequence set forth in SEQ ID NO: 3, and optionally wherein the human transthyretin signal peptide sequence is encoded by a sequence comprising the sequence set forth in SEQ ID NO: 8.

13. The non-human animal of any preceding claim, wherein the entire TTR coding sequence of the endogenous TTR locus has been deleted and replaced with the corresponding human TTR sequence.

14. The non-human animal of claim 13, wherein a region of the endogenous TTR locus from the TTR start codon to the TTR stop codon has been deleted and replaced with the corresponding human TTR sequence.

15. The non-human animal of any preceding claim, wherein a region of the endogenous TTR locus from the TTR start codon to the TTR stop codon has been deleted and replaced with a human TTR sequence comprising the corresponding human TTR sequence and a human TTR 3′ untranslated region,
wherein the endogenous TTR 5′ untranslated region has not been deleted and replaced with the human TTR sequence, and
wherein the humanized endogenous TTR locus comprises an endogenous TTR promoter, wherein the human TTR sequence is operably linked to the endogenous TTR promoter.

16. The non-human animal of any preceding claim, wherein:
(i) the human TTR sequence at the humanized endogenous TTR locus comprises a sequence at least 90%, at least 95%, at least 96%, at least 97%, at least 98%, at least 99%, or 100% identical to the sequence set forth in SEQ ID NO: 24; and/or
(ii) the humanized endogenous TTR locus encodes a transthyretin precursor protein comprising a sequence at least 90%, at least 95%, at least 96%, at least 97%, at least 98%, at least 99%, or 100% identical to the sequence set forth in SEQ ID NO: 2 or encodes a mature transthyretin protein comprising a sequence at least 90%, at least 95%, at least 96%, at least 97%, at least 98%, at least 99%, or 100% identical to the sequence set forth in SEQ ID NO: 5; and/or
(iii) the humanized endogenous TTR locus comprises a transthyretin precursor protein coding sequence comprising a sequence at least 90%, at least 95%, at least 96%, at least 97%, at least 98%, at least 99%, or 100% identical to the sequence set forth in SEQ ID NO: 7 or comprises a mature transthyretin protein coding sequence comprising a sequence at least 90%, at least 95%, at least 96%, at least 97%, at least 98%, at least 99%, or 100% identical to the sequence set forth in SEQ ID NO: 10; and/or
(iv) the humanized endogenous TTR locus comprises a sequence at least 90%, at least 95%, at least 96%, at least 97%, at least 98%, at least 99%, or 100% identical to the sequence set forth in SEQ ID NO: 22 or 23.

17. The non-human animal of any one of claims 1-10, wherein the humanized endogenous TTR locus encodes a transthyretin precursor protein comprising an endogenous transthyretin signal peptide sequence.

18. The non-human animal of claim 17, wherein the endogenous transthyretin signal peptide sequence comprises the sequence set forth in SEQ ID NO: 14, and optionally wherein the endogenous transthyretin signal peptide sequence is encoded by a sequence comprising the sequence set forth in SEQ ID NO: 17.

19. The non-human animal of claim 17 or 18, wherein the first exon of the endogenous TTR locus has not been deleted and replaced with the corresponding human TTR sequence.

20. The non-human animal of claim 19, wherein the first exon and first intron of the endogenous TTR locus have not been deleted and replaced with the corresponding human TTR sequence.

21. The non-human animal of any one of claims 17-20, wherein a region of the endogenous TTR locus from the start of the second TTR exon to the TTR stop codon has been deleted and replaced with the corresponding human TTR sequence.

22. The non-human animal of any one of claims 17-21, wherein a region of the endogenous TTR locus from the second TTR exon to the TTR stop codon has been deleted and replaced with a human TTR sequence comprising the corresponding human TTR sequence and a human TTR 3′ untranslated region,
wherein the endogenous TTR 5′ untranslated region has not been deleted and replaced with the corresponding human TTR sequence, and
wherein the humanized endogenous TTR locus comprises an endogenous TTR promoter, wherein the human TTR sequence is operably linked to the endogenous TTR promoter.

23. The non-human animal of any preceding claim, wherein the humanized endogenous TTR locus does not comprise a selection cassette or a reporter gene.

24. The non-human animal of any preceding claim, wherein the non-human animal is homozygous for the humanized endogenous TTR locus.

25. The non-human animal of any preceding claim, wherein the non-human animal comprises the humanized endogenous TTR locus in its germline.

26. The non-human animal of any preceding claim, wherein the non-human animal is a mammal.

27. The non-human animal of claim 26, wherein the non-human animal is a rat or a mouse.

28. The non-human animal of claim 27, wherein the non-human animal is the mouse.

29. The non-human animal of any preceding claim, wherein serum levels of transthyretin protein expressed from the humanized endogenous TTR in the non-human animal are at least about 20 μg/mL.

30. The non-human animal of any preceding claim, wherein the non-human animal has been seeded with exogenous, pre-formed transthyretin aggregates or fibrils.

31. The non-human animal of claim 30, wherein the exogenous, pre-formed transthyretin aggregates or fibrils comprise a V30M mutation.

32. The non-human animal of claim 30 or 31, wherein the exogenous, pre-formed transthyretin aggregates or fibrils are human.

33. The non-human animal of any one of claim 30-32, wherein the exogenous, pre-formed transthyretin aggregates or fibrils are in the liver, the lung, the heart, the spleen, the kidney, or any combination thereof of the non-human animal.

34. The non-human animal of claim 33, wherein the exogenous, pre-formed transthyretin aggregates or fibrils are in the liver of the non-human animal.

35. The non-human animal of any one of claims 1-34, wherein the non-human animal further comprises in its genome a genomically integrated expression cassette, wherein the genomically integrated expression cassette comprises:
(a) a nucleic acid encoding a chimeric Clustered Regularly Interspaced Short Palindromic Repeats (CRISPR) associated (Cas) protein comprising a nuclease-inactive Cas protein fused to one or more transcriptional activation domains; and
(b) a nucleic acid encoding a chimeric adaptor protein comprising an adaptor protein fused to one or more transcriptional activation domains.

36. The non-human animal of claim 35, further comprising one or more guide RNAs or an expression cassette that encodes the one or more guide RNAs, each guide RNA comprising one or more adaptor-binding elements to which the chimeric adaptor protein can specifically bind,
wherein each of the one or more guide RNAs is capable of forming a complex with the Cas protein and guiding it to a target sequence within a target gene, and
wherein at least one of the one or more guide RNAs targets the humanized endogenous TTR locus.

37. The non-human animal of claim 35 or 36, further comprising a second genomically integrated expression cassette that encodes one or more guide RNAs each comprising one or more adaptor-binding elements to which the chimeric adaptor protein can specifically bind,
wherein each of the one or more guide RNAs is capable of forming a complex with the Cas protein and guiding it to a target sequence within a target gene, and
wherein at least one of the one or more guide RNAs targets the humanized endogenous TTR locus.

38. The non-human animal of any one of claims 35-37, wherein the first expression cassette is integrated into a Rosa26 locus,
the Cas protein is a Cas9 protein comprising mutations corresponding to D10A and N863A when optimally aligned with a Streptococcus pyogenes Cas9 protein,
the one or more transcriptional activator domains in the chimeric Cas protein comprise VP64,
the adaptor protein comprises an MS2 coat protein or a functional fragment or variant thereof,
the one or more transcriptional activation domains in the chimeric adaptor protein comprise p65 and HSF1,
the non-human animal further comprises one or more guide RNAs or an expression cassette that encodes the one or more guide RNAs,
each of the one or more guide RNAs comprises two adaptor-binding elements to which the chimeric adaptor protein can specifically bind,
the two adaptor-binding elements comprise a first adaptor-binding element within a first loop of each of the one or more guide RNAs and a second adaptor-binding element within a second loop of each of the one or more guide RNAs, and
the target sequence is within a region 200 base pairs upstream of the transcription start site and 1 base pair downstream of the transcription start site.

39. A non-human animal cell comprising in its genome a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous Ttr locus comprises a V30M mutation.

40. A non-human animal genome comprising a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous Ttr locus comprises a V30M mutation.

41. A humanized non-human animal TTR gene in which a region of the non-human animal TTR gene comprising both a TTR exonic sequence and a TTR intronic sequence has been deleted and replaced with a corresponding human TTR sequence comprising both a TTR exonic sequence and a TTR intronic sequence, wherein the humanized non-human animal TTR gene comprises a V30M mutation.

42. A targeting vector for generating a humanized endogenous TTR locus in which a region of the endogenous TTR locus comprising both a TTR exonic sequence and a TTR intronic sequence has been deleted and replaced with a corresponding human TTR sequence comprising both a TTR exonic sequence and a TTR intronic sequence, wherein the humanized endogenous TTR locus comprises a V30M mutation, and wherein the targeting vector comprises an insert nucleic acid comprising the V30M mutation and the corresponding human TTR sequence flanked by a 5′ homology arm targeting a 5′ target sequence at the endogenous TTR locus and a 3′ homology arm targeting a 3′ target sequence at the endogenous TTR locus.

43. A method of assessing the activity of a human-TTR-targeting reagent in vivo, comprising:
(a) administering the human-TTR-targeting reagent to the non-human animal of any one of claims 1-38; and
(b) assessing the activity of the human-TTR-targeting reagent in the non-human animal.

44. The method of claim 43, wherein the administering comprises adeno-associated virus (AAV)-mediated delivery, lipid nanoparticle (LNP)-mediated delivery, hydrodynamic delivery (HDD), or injection.

45. The method of claim 44, wherein the administering comprises LNP-mediated delivery.

46. The method of claim 44, wherein the administering comprises AAV8-mediated delivery.

47. The method of any one of claims 43-46, wherein step (b) comprises assessing the activity of the human-TTR-targeting reagent in the liver of the non-human animal.

48. The method of any one of claims 43-47, wherein step (b) comprises measuring expression of a TTR messenger RNA encoded by the humanized endogenous TTR locus.

49. The method of any one of claims 43-48, wherein step (b) comprises measuring expression of a transthyretin protein encoded by the humanized endogenous TTR locus.

50. The method of claim 49, wherein measuring expression of the transthyretin protein comprises measuring serum levels of the transthyretin protein in the non-human animal.

51. The method of claim 49 or 50, wherein measuring expression of the transthyretin protein comprises measuring expression of the transthyretin protein in the liver of the non-human animal.

52. The method of any one of claims 43-51, wherein the human-TTR-targeting reagent is a genome-editing agent, and step (b) comprises assessing modification of the humanized endogenous TTR locus.

53. The method of claim 52, wherein step (b) comprises measuring the frequency of insertions or deletions within the humanized endogenous TTR locus.

54. The method of any one of claims 43-53, wherein the human-TTR-targeting reagent comprises a nuclease agent designed to target a region of a human TTR gene.

55. The method of claim 54, wherein the nuclease agent comprises a Cas protein and a guide RNA designed to target a guide RNA target sequence in the human TTR gene.

56. The method of claim 55, wherein the Cas protein is a Cas9 protein.

57. The method of any one of claims 43-56, wherein the human-TTR-targeting reagent comprises an exogenous donor nucleic acid, wherein the exogenous donor nucleic acid is designed to target the human TTR gene, and optionally wherein the exogenous donor nucleic acid is delivered via AAV.

58. The method of any one of claims 43-51, wherein the human-TTR-targeting reagent is an RNAi agent or an antisense oligonucleotide.

59. The method of any one of claims 43-51, wherein the human-TTR-targeting reagent is an antigen-binding protein.

60. The method of any one of claims 43-51, wherein the human-TTR-targeting reagent is small molecule.

61. The method of any one of claims 43-60, wherein assessing the activity of the human-TTR-targeting reagent in the non-human animal comprises assessing transthyretin activity.

62. The method of any one of claims 43-61, wherein the assessing is in comparison to an untreated control non-human animal.

63. The method of any one of claims 43-62, wherein the method comprises administering exogenous, pre-formed transthyretin aggregates or fibrils to the non-human animal in step (a) or prior to step (a).

64. The method of claim 63, wherein the exogenous, pre-formed transthyretin aggregates or fibrils comprise a V30M mutation.

65. The method of claim 63 or 64, wherein the exogenous, pre-formed transthyretin aggregates or fibrils are human.

66. The method of any one of claims 63-65, wherein the exogenous, pre-formed transthyretin aggregates or fibrils are administered to the non-human animal via intravenous injection.

67. The method of any one of claims 63-66, wherein the exogenous, pre-formed transthyretin aggregates or fibrils are administered via hydrodynamic delivery.

68. The method of any one of claim 63-67, wherein the exogenous, pre-formed transthyretin aggregates or fibrils are administered together with heparin.

69. A method of optimizing the activity of a human-TTR-targeting reagent in vivo, comprising:
(I) performing the method of any one of claims 43-68 a first time in a first non-human animal;
(II) changing a variable and performing the method of step (I) a second time with the changed variable in a second non-human animal; and
(III) comparing the activity of the human-TTR-targeting reagent in step (I) with the activity of the human-TTR-targeting reagent in step (II), and selecting the method resulting in the higher activity.

70. The method of claim 69, wherein the changed variable in step (II) is the delivery vehicle of introducing the human-TTR-targeting reagent into the non-human animal.

71. The method of claim 69, wherein the changed variable in step (II) is the route of administration of introducing the human-TTR-targeting reagent into the non-human animal.

72. The method of claim 69, wherein the changed variable in step (II) is the concentration or amount of the human-TTR-targeting reagent introduced into the non-human animal.

73. The method of claim 69, wherein the changed variable in step (II) is the form of the human-TTR-targeting reagent introduced into the non-human animal.

74. The method of claim 69, wherein the changed variable in step (II) is the human-TTR-targeting reagent introduced into the non-human animal.

75. A method of making the non-human animal of any one of claims 1-34, comprising:
(a) introducing into a non-human animal host embryo a genetically modified non-human animal embryonic stem (ES) cell comprising in its genome a humanized endogenous TTR locus in which a segment of the endogenous TTR locus has been deleted and replaced with a corresponding human TTR sequence, wherein the humanized endogenous TTR locus comprises a V30M mutation; and
(b) gestating the non-human animal host embryo in a surrogate mother, wherein the surrogate mother produces an F0 progeny genetically modified non-human animal comprising the humanized endogenous TTR locus comprising the V30M mutation.

76. The method of claim 75, further comprising modifying the genome of a non-human animal ES cell to comprise the humanized endogenous TTR locus comprising the V30M mutation prior to step (a).

77. A method of making the non-human animal of any one of claims 1-34, comprising:
(a) modifying the genome of a non-human animal one-cell stage embryo to comprise in its genome a humanized endogenous TTR locus comprising a V30M mutation and in which a segment of the endogenous TTR locus has been deleted and replaced with a corresponding human TTR sequence to produce a genetically modified non-human animal embryo;
(b) gestating the genetically modified non-human animal embryo in a surrogate mother, wherein the surrogate mother produces an F0 progeny genetically modified non-human animal comprising the humanized endogenous TTR locus comprising the V30M mutation.

78. The method of any one of claims 75-77, further comprising crossing the F0 progeny genetically modified non-human animal comprising the humanized endogenous TTR locus comprising the V30M mutation with a non-human animal comprising a genomically integrated expression cassette comprising a nucleic acid encoding a chimeric Clustered Regularly Interspaced Short Palindromic Repeats (CRISPR) associated (Cas) protein comprising a nuclease-inactive Cas protein fused to one or more transcriptional activation domains and further comprising a nucleic acid encoding a chimeric adaptor protein comprising an adaptor protein fused to one or more transcriptional activation domains.

79. A method of making the non-human animal of any one of claims 35-38, comprising:
(a) introducing into a non-human animal host embryo a genetically modified non-human animal embryonic stem (ES) cell comprising in its genome: (i) a humanized endogenous TTR locus in which a segment of the endogenous TTR locus has been deleted and replaced with a corresponding human TTR sequence, wherein the humanized endogenous TTR locus comprises a V30M mutation; and (ii) a genomically integrated expression cassette comprising a nucleic acid encoding a Cas protein comprising a nuclease-inactive Cas protein fused to one or more transcriptional activation domains and a nucleic acid encoding a chimeric adaptor protein comprising an adaptor protein fused to one or more transcriptional activation domains; and
(b) gestating the non-human animal host embryo in a surrogate mother, wherein the surrogate mother produces an F0 progeny genetically modified non-human animal comprising the humanized endogenous TTR locus and the genomically integrated expression cassette.

80. The method of claim 79, further comprising modifying the genome of a non-human animal ES cell to comprise the humanized endogenous TTR locus comprising the V30M mutation and the genomically integrated expression cassette prior to step (a).

81. The method of any one of claims 75-80, wherein the non-human animal is a mouse or a rat.

82. The method of claim 81, wherein the non-human animal is a mouse.

83. A method of accelerating transthyretin amyloid deposition in a non-human animal, comprising administering exogenous, pre-formed transthyretin aggregates or fibrils to the non-human animal of any one of claims 1-38.

84. The method of claim 83, wherein the exogenous, pre-formed transthyretin aggregates or fibrils comprise a V30M mutation.

85. The method of claim 83 or 84, wherein the exogenous, pre-formed transthyretin aggregates or fibrils are human.

86. The method of any one of claims 83-85, wherein the exogenous, pre-formed transthyretin aggregates or fibrils are administered to the non-human animal via intravenous injection.

87. The method of any one of claims 83-86, wherein the exogenous, pre-formed transthyretin aggregates or fibrils are administered via hydrodynamic delivery.

88. The method of any one of claims 83-87, wherein the exogenous, pre-formed transthyretin aggregates or fibrils are administered together with heparin.
